SEQUENCE LISTING (40 sequences)

SEQ ID NO: 1 - protein, 116 aa, synthetic construct
MPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQR

SEQ ID NO: 2 - protein, 120 aa, synthetic construct
MAKSGNYSYKRWVFTINNPTFEDYVHVLEFCTLDNCKFAIVGEEKGANGTPHLQGFLNLRSNARAAALEESLGGRAWLSRARGSDEDNEEYCAKESTYLRVGEPVSKGRSSDLAEATSAV

SEQ ID NO: 3 - protein, 207 aa, synthetic construct
MPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPW

SEQ ID NO: 4 - protein, 131 aa, synthetic construct
MAKEKARYFTFLLYPESIPSDWELKLETLGVPMAISPLHDKDKSSIKGQKYKKAHYHVLYIAKNPVTADSVRKKIKLLLGEKSLAMVQVVLNVENMYLYLTHESKDAIAKKKHVYDKADIKLINNFDIDRY

SEQ ID NO: 5 - protein, 330 aa, synthetic construct
MMSIAQVRSAGSAGNYYTDKDNYYVLGSMGERWAGRGAEQLGLQGSVDKDVFTRLLEGRLPDGADLSRMQDGSNRHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQTIREAVGEDASLKSRDVAALDTRKSKQHVDPEIKMAEWMQTLKETGFDIRAYRDAADQRADLRTLTPGPASQDGPDVQQAVTQAIAGLSER

SEQ ID NO: 6 - protein, 186 aa, synthetic construct
MAIYHLTAKTGSRSGGQSARAKADYIQREGKYARDMDEVLHAESGHMPEFVERPADYWDAADLYERANGRLFKEVEFALPVELTLDQQKALASEFAQHLTGAERLPYTLAIHAGGGENPHCHLMISERINDGIERPAAQWFKRYNGKTPEKGGAQKTEALKPKAWLEQTREAWADHANRALERAGH

SEQ ID NO: 7 - protein, 220 aa, synthetic construct
MAMYHFQNKFVSKANGQSATAKSAYNSASRIKDFKENEFKDYSNKQCDYSEILLPNNADDKFKDREYLWNKVHDVENRKNSQVAREIIIGLPNEFDPNSNIELAKEFAESLSNEGMIVDLNIHKINEENPHAHLLCTLRGLDKNNEFEPKRKGNDYIRDWNTKEKHNEWRKRWENVQNKHLEKNGFSVRVSADSYKNQNIDLEPTKKEGWKARKFEDETG

SEQ ID NO: 8 - DNA, 15 bp, synthetic construct
  source 1..15: mol_type = other DNA
aagtattaccagaaa

SEQ ID NO: 9 - DNA, 12 bp, synthetic construct
  source 1..12: mol_type = other DNA
aagtattaccag

SEQ ID NO: 10 - DNA, 20 bp, synthetic construct
  source 1..20: mol_type = other DNA
caacggtatatatcctgcca

SEQ ID NO: 11 - DNA, 23 bp, synthetic construct
  source 1..23: mol_type = other DNA
tgcttccgtactacgacccccca

SEQ ID NO: 12 - DNA, 22 bp, synthetic construct
  source 1..22: mol_type = other DNA
tttgcgtggggtgtggtgcttt

SEQ ID NO: 13 - DNA, 38 bp, synthetic construct
  source 1..38: mol_type = other DNA
ccagtttctcgaagagaaaccggtaagtgcaccctccc

SEQ ID NO: 14 - DNA, 45 bp, synthetic construct
  source 1..45: mol_type = other DNA
acgcgaacggaacgttcgcataagtgcgcccttacgggatttaac

SEQ ID NO: 15 - DNA, 61 bp, synthetic construct
  source 1..61: mol_type = other DNA
aagtattaccagacgtgtgctcttccgatctgagggtacttagatcggaagagcgtcgtgt

SEQ ID NO: 16 - DNA, 61 bp, synthetic construct
  source 1..61: mol_type = other DNA
aagtattaccagacgtgtgctcttccgatctgacaattgccagatcggaagagcgtcgtgt

SEQ ID NO: 17 - DNA, 61 bp, synthetic construct
  source 1..61: mol_type = other DNA
aagtattaccagacgtgtgctcttccgatcttaacgcacctagatcggaagagcgtcgtgt

SEQ ID NO: 18 - DNA, 61 bp, synthetic construct
  source 1..61: mol_type = other DNA
aagtattaccagacgtgtgctcttccgatctatgatcgtcgagatcggaagagcgtcgtgt

SEQ ID NO: 19 - DNA, 61 bp, synthetic construct
  source 1..61: mol_type = other DNA
aagtattaccagacgtgtgctcttccgatcttgtctcccatagatcggaagagcgtcgtgt

SEQ ID NO: 20 - DNA, 20 bp, synthetic construct
  source 1..20: mol_type = other DNA
taccacaaggtgctccatca

SEQ ID NO: 21 - DNA, 20 bp, synthetic construct
  source 1..20: mol_type = other DNA
tatcaagccccgactacctg

SEQ ID NO: 22 - DNA, 21 bp, synthetic construct
  source 1..21: mol_type = other DNA
actgtctgttttccttgttgg

SEQ ID NO: 23 - DNA, 18 bp, synthetic construct
  source 1..18: mol_type = other DNA
aacgggtttcctggttgg

SEQ ID NO: 24 - DNA, 70 bp, synthetic construct
  source 1..70: mol_type = other DNA
aatgatacggcgaccaccgagatctacacaggctataacactctttccctacacgacgctcttccgatct

SEQ ID NO: 25 - DNA, 33 bp, synthetic construct
  source 1..33: mol_type = other DNA
tgactggagttcagacgtgtgctcttccgatct

SEQ ID NO: 26 - DNA, 54 bp, synthetic construct
  source 1..54: mol_type = other DNA
caagcagaagacggcatacgagatggcagatagtgactggagttcagacgtgtg

SEQ ID NO: 27 - protein, 361 aa, synthetic construct
EVQLQQSGPELVKPGTSMNISCKASGYTFTNYYMHWVRQRPGQGLEWIGWIYPGDGNTHYNEKFKGKTTLTADTSSITAYMLLSSLTSEDSAVYFCARDNSGYVGFPYWGQGTTLTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGGGSGGGSGGGSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRPKKKRKVHHHHHH

SEQ ID NO: 28 - protein, 219 aa, synthetic construct
DIKMTQSPSSLTVTAGERVTMSCKSSQSLFNSANQENYLTWYQQRPGQPPKLLIYWASTRDSGVPDRFTGSGSGTDFPLTISSVQAEDLAVYYCQNDYSYPLTFGAGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 29 - protein, 360 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGGGSGGGSGGGSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRPKKKRKVHHHHHH

SEQ ID NO: 30 - protein, 214 aa, synthetic construct
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 31 - protein, 230 aa, synthetic construct
MASGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRPKKKRKVHHHHHH

SEQ ID NO: 32 - DNA, 702 bp, synthetic construct
  source 1..702: mol_type = other DNA
catatggcgagcggcagcgatagcgaagtgaaccaagaagcgaaaccggaagtgaagccggaagtgaagccggaaacccatattaacctgaaagtgagcgatggcagcagcgaaattttttttaaaattaaaaaaaccaccccgctgcgccgcctgatggaagcgtttgcgaaacgccaaggcaaagaaatggatagcctgcgctttctgtatgatggcattcgcattcaagcggatcagaccccggaagatctggatatggaagataacgatattattgaagcgcatcgcgaacagattggcggcagcccgagcaaaaaaaacggccgcagcggcccgcagccgcataaacgctgggtgtttaccctgaacaacccgagcgaagatgaacgcaagaaaattcgcgatctgccgattagcctgtttgattattttattgtgggcgaagaaggcaacgaagaaggccgcaccccgcatctgcaaggctttgcgaactttgtgaaaaaacagacctttaacaaagtgaaatggtatctgggcgcgcgctgccatattgaaaaagcgaaaggcaccgatcagcagaacaaagaatattgcagcaaagaaggcaacctgctgatggaatgcggcgcgccgcgcagccaaggtcagcgcccgaaaaagaaacgtaaagtgcatcaccatcaccatcattaactcgag

SEQ ID NO: 33 - DNA, 1207 bp, synthetic construct
  source 1..1207: mol_type = other DNA
ccatggcgcacccttagcgagaggtttatcattaaggtcaacctctggatgttgtttcggcatcctgcattgaatctgagttactgtctgttttccttgttggaacggagagcatcacacgacgctcttccgatctaagtaccctcagatcggaagagcacacgtctggtaatacttgatgctctccgagccaaccaggaaacccgttttttctgacgtaagggtgcgcaactttcatgaaatccgctgaatatttgaacacttttagattgagaaatctcggcctacctgtcatgaacaatttgcatgacatgtctaaggcgactcgcatatctgttgaaacacttcggttgttaatctatacagctgattttcgctataggatctacactgtagaaaagaaaggcccagagaagagaatgagaaccatttaccaaccttctcgagaacttaaagccttacaaggatgggttctacgtaacattttagataaactgtcgtcatctcctttttctattggatttgaaaagcaccaatctattttgaataatgctaccccgcatattggggcaaactttatactgaatattgatttggaggattttttcccaagtttaactgctaacaaagtttttggagtgttccattctcttggttataatcgactaatatcttcagttttgacaaaaatatgttgttataaaaatctgctaccacaaggtgctccatcatcacctaaattagctaatctaatatgttctaaacttgattatcgtattcagggttatgcaggtagtcggggcttgatatatacgagatatgccgatgatctcaccttatctgcacagtctatgaaaaaggttgttaaagcacgtgattttttattttctataatcccaagtgaaggattggttattaactcaaaaaaaacttgtattagtgggcctcgtagtcagaggaaagttacaggtttagttatttcacaagagaaagttgggataggtagagaaaaatataaagaaattagagcaaagatacatcatatattttgcggtaagtcttctgagatagaacacgttaggggatggttgtcatttattttaagtgtggattcaaaaagccataggagattaataacttatattagcaaattagaaaaaaaatatggaaagaaccctttaaataaagcgaagacctaagcggccgc

SEQ ID NO: 34 - DNA, 1200 bp, synthetic construct
  source 1..1200: mol_type = other DNA
ccatggaaatccgctgaatatttgaacacttttagattgagaaatctcggcctacctgtcatgaacaatttgcatgacatgtctaaggcgactcgcatatctgttgaaacacttcggttgttaatctatacagctgattttcgctataggatctacactgtagaaaagaaaggcccagagaagagaatgagaaccatttaccaaccttctcgagaacttaaagccttacaaggatgggttctacgtaacattttagataaactgtcgtcatctcctttttctattggatttgaaaagcaccaatctattttgaataatgctaccccgcatattggggcaaactttatactgaatattgatttggaggattttttcccaagtttaactgctaacaaagtttttggagtgttccattctcttggttataatcgactaatatcttcagttttgacaaaaatatgttgttataaaaatctgctaccacaaggtgctccatcatcacctaaattagctaatctaatatgttctaaacttgattatcgtattcagggttatgcaggtagtcggggcttgatatatacgagatatgccgatgatctcaccttatctgcacagtctatgaaaaaggttgttaaagcacgtgattttttattttctataatcccaagtgaaggattggttattaactcaaaaaaaacttgtattagtgggcctcgtagtcagaggaaagttacaggtttagttatttcacaagagaaagttgggataggtagagaaaaatataaagaaattagagcaaagatacatcatatattttgcggtaagtcttctgagatagaacacgttaggggatggttgtcatttattttaagtgtggattcaaaaagccataggagattaataacttatattagcaaattagaaaaaaaatatggaaagaaccctttaaataaagcgaagacctaaactttcagattccgtatgcgcacccttagcgagaggtttatcattaaggtcaacctctggatgttgtttcggcatcctgcattgaatctgagttactgtctgttttccttgttggacacgacgctcttccgatctaagtaccctcagatcggaagagcacacgtctggtaatacttccaaccaggaaacccgttttttctgacgtaagggtgcgcatacggaatctgcggccgc

SEQ ID NO: 35 - protein, 6 aa, synthetic construct
GGGGSX

SEQ ID NO: 36 - protein, 6 aa, synthetic construct
EAAAKX

SEQ ID NO: 37 - DNA, 10 bp, synthetic construct
  source 1..10: mol_type = other DNA
aagtattacc

SEQ ID NO: 38 - DNA, 1196 bp, synthetic construct
  source 1..1196: mol_type = other DNA
atgcgcacccttagcgagaggtttatcattaaggtcaacctctggatgttgtttcggcatcctgcattgaatctgagttactgtctgttttccttgttggaacggagagcatcacacgacgctcttccgatctaagtaccctcagatcggaagagcacacgtctggtaatacttgatgctctccgagccaaccaggaaacccgttttttctgacgtaagggtgcgcaactttcatgaaatccgctgaatatttgaacacttttagattgagaaatctcggcctacctgtcatgaacaatttgcatgacatgtctaaggcgactcgcatatctgttgaaacacttcggttgttaatctatacagctgattttcgctataggatctacactgtagaaaagaaaggcccagagaagagaatgagaaccatttaccaaccttctcgagaacttaaagccttacaaggatgggttctacgtaacattttagataaactgtcgtcatctcctttttctattggatttgaaaagcaccaatctattttgaataatgctaccccgcatattggggcaaactttatactgaatattgatttggaggattttttcccaagtttaactgctaacaaagtttttggagtgttccattctcttggttataatcgactaatatcttcagttttgacaaaaatatgttgttataaaaatctgctaccacaaggtgctccatcatcacctaaattagctaatctaatatgttctaaacttgattatcgtattcagggttatgcaggtagtcggggcttgatatatacgagatatgccgatgatctcaccttatctgcacagtctatgaaaaaggttgttaaagcacgtgattttttattttctataatcccaagtgaaggattggttattaactcaaaaaaaacttgtattagtgggcctcgtagtcagaggaaagttacaggtttagttatttcacaagagaaagttgggataggtagagaaaaatataaagaaattagagcaaagatacatcatatattttgcggtaagtcttctgagatagaacacgttaggggatggttgtcatttattttaagtgtggattcaaaaagccataggagattaataacttatattagcaaattagaaaaaaaatatggaaagaaccctttaaataaagcgaagacctaa

SEQ ID NO: 39 - DNA, 21 bp, synthetic construct
  source 1..21: mol_type = other DNA
agacgtgtgctcttccgatct

SEQ ID NO: 40 - DNA, 20 bp, synthetic construct
  source 1..20: mol_type = other DNA
acacgacgctcttccgatct

(19) United States
(12) Patent Application Publication
(10) Pub. No.: US 2025/0197844 A1
(43) Pub. Date: Jun. 19, 2025

(54) METHODS AND COMPOSITIONS FOR TARGETED DELIVERY OF INTRACELLULAR BIOLOGICS

(71) Applicant: Travin Bio, Inc., Cambridge, MA (US)

(72) Inventors: Maria G. Beconi, Bedford, MA (US); Vu Phong Hong, Cambridge, MA (US)

(21) Appl. No.: 18/835,968

(22) PCT Filed: Feb. 7, 2023
(86) PCT No.: PCT/US2023/062160

§ 371 Date: Aug. 5, 2024

Related U.S. Application Data
(60) Provisional application No. 63/307,321, filed on Feb. 7, 2022.

(51) Int. Cl. C12N 15/10; C07K 16/28; C12N 15/70; C12Q 1/6897; C40B 40/08

(52) U.S. Cl. CPC C12N 15/1065; C07K 16/2881; C12N 15/70; C12Q 1/6897; C40B 40/08; C07K 2319/80; C12Q 2600/136; C12Q 2600/178

(57) ABSTRACT

Provided herein are methods and compositions for generating a pool of polypeptide variants, each with a covalently attached unique single-stranded DNA (ssDNA) barcode that allows screening and selection of the associated polypeptide variants.

### CLAIM OF PRIORITY

This application claims the benefit of U.S. Provisional Application Ser. No. 63/307,321, filed on Feb. 7, 2022. The entire contents of the foregoing are incorporated herein by reference.

### SEQUENCE LISTING

This application contains a Sequence Listing that has been submitted electronically as an XML file named “53070-0002WO1_SL ST26.XML.” The XML file, created on Feb. 3, 2023, is 43,169 bytes in size. The material in the XML file is hereby incorporated by reference in its entirety.

### TECHNICAL FIELD

This disclosure generally relates to methods and compositions for delivery of polypeptides.

### BACKGROUND

Biologics, including, antisense oligonucleotides (ASO) and small interfering RNAs (siRNAs), are commonly evaluated and used as therapies to reduce or increase the abundance/translation of selected RNAs that are either under or over expressed in the disease state, most commonly due to genetic defects. In general, biologics as therapeutics have two key shortcomings: (1) low uptake into the tissue and/or cell of interest; and (2) lack of specificity which lowers the potential therapeutic index that could be achieved if the delivery of the biologic was targeted to the tissue and/or cell of interest. These shortcomings have been overcome with the use of delivery agents (peptides, nanoparticles, antibody fragments, or others) that target internalized receptors in the tissue or cell of interest, and that when conjugated to the biologic (cargo), results in increased and selective delivery of the cargo to cells or tissues of interest, when compared to unconjugated biologics. Optimization of the delivery agents is a key component of success for increased and selective delivery of cargo. Screening and optimization for delivery agents is typically performed in two steps. The first step is in vitro-in cell culture, individual wells, and/or vials, with known and characterized delivery agents. The delivery agents that meet pre-specified criteria for optimization in vitro are then evaluated in vivo, where each delivery agent has to be evaluated in a different animal. This process is inefficient and allows only a small subset of many possible variants of delivery agents to be tested. Moreover, the in vitro results can be a poor predictor of in vivo effects, and in vitro screens cannot be designed to predict whole-body bio-distribution, which might drive off-target toxicity (see, e.g., Whitehead et al., ACS Nano 6 (8): 6922-6929, 2012; Dirin et al., Expert Opin Biol Ther 13 (6): 875-88, 2013). Thus, there is need in the art for methods that would allow more efficient screening and optimization for delivery agents.

### SUMMARY

The present disclosure provides methods and compositions for generating a pool of polypeptide variants, each with a covalently attached unique single-stranded DNA (ssDNA) barcode.

In a first aspect, disclosed herein is an expression construct comprising: (i) a nucleic acid sequence encoding for a fusion protein, wherein the fusion protein comprises a protein of interest (POI) and a DNA binding protein (DBP) that is fused to the POI; (ii) a nucleic acid sequence encoding for a single-stranded DNA (ssDNA), wherein the ssDNA comprises an ssDNA recognition sequence corresponding to the DBP, and a unique ssDNA barcode corresponding to the POI; and (iii) one or more promoters to drive expression of the fusion protein and the ssDNA. In preferred embodiments, upon expression of the fusion protein and the ssDNA, the ssDNA recognition sequence binds to the DBP in the fusion protein. In some embodiments, also provided herein are compositions wherein the nucleic acid sequence encoding for the fusion protein and the nucleic acid sequence encoding for a ssDNA are in separate expression constructs, e.g., in a single combined composition, i.e., mixed together, or are separate, e.g., in a kit; although this summary refers to expression constructs, such compositions and kits are encompassed by the embodiments described.

In some embodiments, the DBP is fused to the N-terminal or C-terminal of the POI. In certain embodiments, the DBP is fused to the N-terminal or C-terminal of the POI with a linker therebetween.

In some embodiments, the DBP is a HUH endonuclease.

In some instances, the DBP comprises an amino acid sequence having at least 85% sequence identity to the amino acid sequence set forth in any one of SEQ ID NOs: 1-7. In certain instances, the DBP comprises an amino acid sequence having at least 90% sequence identity to the amino acid sequence set forth in any one of SEQ ID NOs: 1-7. In particular instances, the DBP comprises the amino acid sequence set forth in any one of SEQ ID NOs: 1-7.

In some embodiments, the POI comprises an antibody or antigen-binding fragment thereof, preferably an antigen-binding fragment (Fab) comprising a heavy chain variable (VH) domain and/or a light chain variable (VL) domain.

In some embodiments, the antibody or antigen-binding fragment thereof comprises a VH domain comprising an amino acid sequence having at least 85% sequence identity to the amino acid sequence set forth in SEQ ID NO: 27 or 29; and/or a VL domain comprising an amino acid sequence having at least 85% sequence identity to the amino acid sequence set forth in SEQ ID NO: 28 or 30. In certain embodiments, the antibody or antigen-binding fragment thereof comprises a VH domain comprising an amino acid sequence having at least 90% sequence identity to the amino acid sequence set forth in SEQ ID NO: 27 or 29; and/or a VL domain comprising an amino acid sequence having at least 90% sequence identity to the amino acid sequence set forth in SEQ ID NO: 28 or 30. In particular embodiments, the antibody or antigen-binding fragment thereof comprises a VH domain comprising the amino acid sequence set forth in SEQ ID NO: 27 or 29; and/or a VL domain comprising the amino acid sequence set forth in SEQ ID NO: 28 or 30.

In some embodiments, the nucleic acid sequence encoding a ssDNA further comprises a non-coding RNA (ncRNA), wherein the nucleic acid sequence encoding the ssDNA is inserted into the ncRNA. In certain instances, the nucleic acid sequence encoding the ssDNA is transcribed into an ssDNA product that contains the ssDNA recognition sequence and the unique ssDNA barcode. In some instances, the ncRNA comprises a stem loop structure, and sequence encoding the ssDNA is inserted into the stem loop structure.

In some embodiments, the expression construct further comprises a sequence encoding a reverse transcriptase (RT) that is compatible with the ncRNA. In some instances, the expression construct comprises the sequence encoding the RT at the 5′ or 3′ end of the ncRNA.

In some instances, the ncRNA and RT is derived from Retron-Eco1 (Ec86), Retron-Eco2 (Ec67), Retron-Eco3 (Ec73), Retron-Eco4 (Ec83), Retron-Eco5 (Ec107), Retron-Eco6 (Ec48), Retron-Eco7 (Ec78), Retron-Mxa1 (Mx162), Retron-Mxa2 (Mx65), Retron-Sau1 (Sa163), Retron-Nex1 (Ne160), Retron-Nex2 (Ne144), Retron-Sen1 (Se72), Retron-Sen2 (St85), Retron-Vch1 (Vc95), Retron-Vch2 (Vc81), Retron-Vch3 (Vc137), Retron-Vpa1 (Vp96), Retron-Kpn1, Retron-Pmi1, Retron-Rxx1, Retron-Bxx1, Retron-Fel1, Retron-Cco1, Retron-Adi1, Retron-Age1, Retron-Cfe1, Retron-Cfu1, Retron-Cvi1, Retron-Mli1, Retron-Mco1, Retron-Mfu1, Retron-Mma1, Retron-Mst1, Retron-Mvi1, Retron-Cap1, Retron-Cpe1, or Retron-Sce1.

In some embodiments, the ncRNA comprises nucleotides 6-116 and nucleotides 178-229 of SEQ ID NO: 33; optionally in a first part comprising nucleotides 6-116 and a second part comprising nucleotides 178-229 of SEQ ID NO: 33, wherein the nucleic acid sequence encoding the ssDNA is inserted into the ncRNA between the first and second parts.

In some embodiments, the ncRNA comprises nucleotides 2-113 and nucleotides 175-227 of SEQ ID NO: 38; optionally in a first part comprising nucleotides 2-113 and a second part comprising nucleotides 175-227 of SEQ ID NO: 38, wherein the nucleic acid sequence encoding the ssDNA is inserted into the ncRNA between the first and second parts.

In some embodiments, the sequence encoding the RT comprises nucleotides 237-1199 of SEQ ID NO: 33 or nucleotides 234-1196 of SEQ ID NO: 38.

In some embodiments, the expression construct comprises nucleotides 6-116, nucleotides 178-229, and/or nucleotides 237-1199 of SEQ ID NO: 33. In some embodiments, the expression construct comprises nucleotides 2-113, nucleotides 175-227, and/or nucleotides 234-1196 of SEQ ID NO: 38.

In some instances, the ssDNA recognition sequence comprises a nucleic acid sequence having at least 85% sequence identity to the nucleic acid sequence set forth in any one of SEQ ID NOs: 8-14. In certain instances, the ssDNA recognition sequence comprises a nucleic acid sequence having at least 90% sequence identity to the nucleic acid sequence set forth in any one of SEQ ID NOs: 8-14. In particular instances, the ssDNA recognition sequence comprises the nucleic acid sequence set forth in any one of SEQ ID NOs: 8-14.

In some instances, the ssDNA barcode comprises a nucleic acid sequence having at least 85% sequence identity to the nucleic acid sequence set forth in any one of SEQ ID NOs: 15-19. In certain instances, the ssDNA barcode comprises a nucleic acid sequence having at least 90% sequence identity to the nucleic acid sequence set forth in any one of SEQ ID NOs: 15-19. In particular instances, the ssDNA barcode comprises the nucleic acid sequence set forth in any one of SEQ ID NOs: 15-19.

In some embodiments, the expression construct comprises one single promoter to drive expression of the fusion protein and the ssDNA. In other embodiments, the expression construct comprises at least two promoters, e.g., wherein a first promoter drives expression of the fusion protein and a second promoter drives expression of the SSDNA.

In some embodiments, the promoter (e.g., the single promoter, the first promoter and/or the second promoter) is selected from the group consisting of T7, T7lac, lac, Sp6, araBAD, trp, Ptac, pL, T3CMV, SV40, EF1a, PGK1, Ubc, human beta actin, CAG, TRE, UAS, Ac5, POlyhedrin, CaMKIIa, GAL-1,10, TEF1, GDS, ADH1, CaMV35S, H1, and U6.

In some embodiments, the expression construct further comprise a nucleic acid sequence encoding for a purification tag. In certain embodiments, the purification tag is a His-tag. In other embodiments, the purification tag is a FLAG tag or a biotin-tag.

In another aspect, the present disclosure provides an isolated cell comprising the expression construct described hereinabove.

In some embodiments, the cell is a prokaryotic cell or a eukaryotic cell. In certain embodiments, the cell is Escherichia coli, Saccharomyces cerevisiae, an insect cell, or a mammalian cell.

In yet another aspect, the present disclosure provides a method of generating a DNA encoded polypeptide (DEP) by: (a) transforming an expression construct described hereinabove into cells under conditions in which one expression construct (e.g., at least one (e.g., 1, 2, 3, 4, 5, 6, 7, 8, 9, 10, 11, 12, 13, 14, 15, 16, 17, 18, 19, 20, or more) expression construct) is introduced into each cell; and (b) culturing the cells under conditions in which the expression construct is expressed, and the DBP of the fusion protein binds to the corresponding ssDNA recognition sequence, thereby producing a DEP.

In some embodiments of the above aspect, the method further comprises purifying the DEP. In certain embodiments, the purifying step comprises pulling down the DEP with a pull-down assay, wherein the pull-down assay is compatible with the purification tag that is encoded by the expression construct.

In some embodiments, the DEP comprises the fusion protein and the ssDNA, wherein the fusion protein is conjugated to the ssDNA by a covalent bond or a non-covalent bond. In certain embodiments, the covalent bond or the non-covalent bond is between the DBP of the fusion protein and its corresponding ssDNA recognition sequence.

In some embodiments, the method further comprises identifying the POI of the fusion protein. In certain embodiments, the identifying step comprises sequencing the ssDNA barcode.

In some embodiments, the cell is a prokaryotic cell or a eukaryotic cell. In certain embodiments, the cell is Escherichia coli, Saccharomyces cerevisiae, an insect cell, or a mammalian cell.

In another aspect, the present disclosure provides a DEP generated by the method described hereinabove.

In yet another aspect, the present disclosure provides a composition comprising the DEP described hereinabove.

In some embodiments, the composition further comprises a pharmaceutically acceptable carrier.

In another aspect, the present disclosure provides a method of generating a DEP library by: (a) transforming a pool of expression constructs into cells under conditions in which one expression construct (e.g., at least one (e.g., 1, 2, 3, 4, 5, 6, 7, 8, 9, 10, 11, 12, 13, 14, 15, 16, 17, 18, 19, 20, or more) expression construct) is introduced into each cell, wherein the pool of expression constructs comprises a plurality of an expression construct described hereinabove; and (b) culturing the cells under conditions in which the pool of expression constructs are expressed, and the DBP of the fusion proteins bind to the corresponding ssDNA recognition sequences, thereby producing a DEP library.

In another aspect, the present disclosure provides a DEP library generated by the method described hereinabove.

In some embodiments, the DEP library comprise about 10² to about 10¹⁴ DEPs.

Also disclosed is a DEP from the DEP library described hereinabove, wherein the DEP comprises a fusion protein conjugated to an ssDNA, wherein the fusion protein comprises a POI variant and a DBP fused to the POI variant; the ssDNA comprises an ssDNA recognition sequence corresponding to the DBP, and a unique ssDNA barcode corresponding to the POI variant; and the DBP of the fusion protein is conjugated to the ssDNA recognition sequence of the ssDNA by a covalent bond or a non-covalent bond.

In another aspect, the present disclosure provides a method for selecting a polypeptide variant as a candidate delivery vehicle by: (a) generating a DEP library by a method described hereinabove, wherein the pool of expression constructs comprises nucleic acid sequences encoding for a pool of polypeptide variants, and wherein each DEP of the DEP library comprises a fusion protein conjugated to an ssDNA, wherein the fusion protein comprises a polypeptide variant and a DBP fused to the polypeptide variant, and the ssDNA comprises an ssDNA recognition sequence corresponding to the DBP and a unique ssDNA barcode corresponding to the polypeptide variant; (b) administering the DEP library generated in step (a) to an animal; (c) obtaining a biological sample from the animal; (d) processing the biological sample by homogenization or subcellular fractionation to extract the DEP; (e) next generation sequencing to identify the ssDNA barcodes and the corresponding polypeptide variants; (f) screening a tissue and/or cellular fraction and/or subcellular fraction of interest obtained in step (d) to determine abundance of ssDNA barcodes and the corresponding polypeptide variants therein; and (g) selecting a polypeptide variant as a candidate delivery vehicle based on abundance of ssDNA barcode specific for the polypeptide variant in the tissue and/or cellular fraction and/or subcellular fraction of interest.

In some embodiments of the above aspect, the animal is a non-human mammal. In certain embodiments, the animal is a non-human primate, a mouse, a rat, a rabbit, a mini-pig, a sheep, or a dog.

In some embodiments of the above aspect, the administration to the animal is by enteral administration, parenteral administration, intranasal administration, administration by inhalation, or vaginal administration. In certain embodiments, the enteral administration is by intravenous injection, intramuscular injection, subcutaneous injection, topical administration, or transdermal administration.

In some embodiments of the above aspect, the biological sample is a tissue, blood and/or plasma.

In another aspect, the present disclosure provides an in vitro method for selecting a polypeptide variant as a candidate delivery vehicle by: (a) generating a DEP library by a method described hereinabove, wherein the pool of expression constructs comprises nucleic acid sequences encoding for a pool of polypeptide variants, and wherein each DEP of the DEP library comprises a fusion protein conjugated to an ssDNA, wherein the fusion protein comprises a polypeptide variant and a DBP fused to the polypeptide variant, and the ssDNA comprises an ssDNA recognition sequence corresponding to the DBP and a unique ssDNA barcode corresponding to the polypeptide variant; (b) dosing the DEP library to a cell culture; (c) processing the cell culture by homogenization or subcellular fractionation to extract the DEP; (e) next generation sequencing to identify the ssDNA barcodes and the corresponding polypeptide variants; (f) screening a cellular fraction and/or subcellular fraction of interest obtained in step (c) to determine abundance of ssDNA barcodes and the corresponding polypeptide variants therein; and (g) selecting a polypeptide variant as a candidate delivery vehicle based on abundance of ssDNA barcode specific for the polypeptide variant in the cellular fraction and/or subcellular fraction of interest.

In some embodiments, the polypeptide variant is selected as a candidate delivery vehicle for a bio-therapeutic. In certain embodiments, the bio-therapeutic is an antisense oligonucleotides (ASO) and/or a small interfering RNA (siRNA).

In some embodiments, the polypeptide variant is a variant of a GLP1R (glucagon like peptide 1 receptor) binder, a DPP6 (dipeptidyl peptidase like 6) binder, and/or a CCK-2 (cholecystokinin-2 receptor) binder.

In another aspect, the present disclosure provides a method for selecting a polypeptide variant as a candidate bio-therapeutic by: (a) generating a DEP library by a method described hereinabove, wherein the pool of expression constructs comprises nucleic acid sequences encoding for a pool of polypeptide variants, and wherein each DEP of the DEP library comprises a fusion protein conjugated to an ssDNA, wherein the fusion protein comprises a polypeptide variant and a DBP fused to the polypeptide variant, and the ssDNA comprises an ssDNA recognition sequence corresponding to the DBP and a unique ssDNA barcode corresponding to the polypeptide variant; (b) administering the DEP library generated in step (a) to an animal; (c) obtaining plasma sample from the animal at a specific time-point after administration of the DEP library; (d) processing the plasma sample by homogenization to extract the DEP; (e) next generation sequencing to identify the ssDNA barcodes and the corresponding polypeptide variants; (f) screening the homogenized plasma sample obtained in step (d) to determine abundance of ssDNA barcodes and the corresponding polypeptide variants in the plasma; and (g) selecting a polypeptide variant as a candidate bio-therapeutic based on abundance of ssDNA barcode specific for the polypeptide variant in the plasma.

In some embodiments of the above aspect, the animal is a non-human mammal. In certain embodiments, the animal is a non-human primate, a mouse, a rat, a rabbit, a mini-pig, a sheep, or a dog.

In some embodiments of the above aspect, the administration to the animal is by enteral administration, parenteral administration, intranasal administration, administration by inhalation, or vaginal administration. In certain embodiments, the enteral administration is by intravenous injection, intramuscular injection, subcutaneous injection, topical administration, or transdermal administration.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic representation of methods for generating polypeptide and single stranded DNA conjugates in cells.

FIG. 2 is a schematic representation of DNA encoded polypeptide (DEP) library generation.

FIG. 3 is a schematic representation of application of DEP library for screening and identification of cell/tissue targeted delivery vehicles for biologics, including proteins and oligonucleotides.

FIG. 4 is a schematic representation of intracellular production of SUMO-PCV-ssDNA conjugate with TRV-R-001.

FIG. 5 is a schematic representation of intracellular production of SUMO-PCV-ssDNA conjugate with TRV-R-002.

FIGS. 6A-6C provide cartoons that schematically represent generation of a library of GLP1 peptides and barcodes.

FIG. 7 provides cartoons that schematically represent expression of a DEP library containing GLP1 peptides and barcodes.

FIGS. 8A-8B provide cartoons that schematically represent delivery of ssDNA barcodes into K562 cells with a TfR1 targeting complex.

FIG. 9 provides representative image of SDS-PAGE and western blot analyses of SUMO-PCV reaction with ssDNA and shows the formation of ssDNA-SUMO-PCV conjugate.

FIG. 10 is a bar graph showing enrichment of RT-DNA/plasmid template over the plasmid alone by qPCR, relative to uninduced condition.

FIG. 11 is a bar graph showing presence of barcode in the eluted fraction from his-tag purification, as confirmed by qPCR.

### DETAILED DESCRIPTION

The present disclosure provides methods and compositions that allow a pool of (e.g., about 10² to about 10¹⁴) polypeptide variants to be made, each with a covalently attached unique single-stranded DNA (ssDNA) barcode. The unique barcode enables simultaneous screening and selection of polypeptide variants in vivo (e.g., in one animal) and/or in vitro (e.g., in one well), followed by identification of the polypeptide variant using its unique barcode.

### References and Definitions

The present disclosure now will be described more fully hereinafter. The disclosure may be embodied in many different forms and should not be construed as limited to the aspects set forth herein; rather, these aspects are provided so that this disclosure will satisfy applicable legal requirements.

As used herein, “a,” “an,” or “the” can mean one or more than one. For example, “a” polypeptide can mean a single polypeptide or a multiplicity of polypeptides.

As used herein, unless specifically indicated otherwise, the word “or” is used in the inclusive sense of “and/or” and not the exclusive sense of “either/or.”

The term “about” or “approximately” usually means within 5%, or more, preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence (e.g., an mRNA sequence), a complementary polynucleotide sequence (cDNA), a genomic polynucleotide sequence and/or a composite polynucleotide sequences (e.g., a combination of the above).

As used herein, the term “expression” or “expressing” refers to the transcription and/or translation of a particular nucleotide sequence driven by a promoter. As used herein, the term “endogenous” in reference to a gene or nucleotide sequence or protein is intended to mean a gene or nucleotide sequence or protein that is naturally comprised within or expressed by a cell. Endogenous genes can include genes that naturally occur in the cell of a plant or animal, but that have been modified in the genome of the cell without insertion or replacement of a heterologous gene that is from another plant or animal species or another location within the genome of the modified cell.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol.266:131-141; Altschul et al. (1997), Nucleic Acids Res. 25:3389-3402); Zhang et al. (2000), J. Comput. Biol. 7 (1-2): 203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. (Proc Natl Acad Sci 89:10915-9 (1992)). Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof.

Amino acid sequences described herein may include “conservative mutations,” including the substitution, deletion or addition of nucleic acids that alter, add or delete a single amino acid or a small number of amino acids in a coding sequence where the nucleic acid alterations result in the substitution of a chemically similar amino acid. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N) and glutamine (Q); N, Q, serine(S), threonine (T), and tyrosine (Y); K, R, H, D, and E; D, E, N, and Q; alanine (A), valine (V), leucine (L), isoleucine (I), proline (P), phenylalanine (F), tryptophan (W), methionine (M), cysteine (C), and glycine (G); F, W, and Y; H, F, W, and Y; C, S and T; C and A; S and T; C and S; S, T, and Y; V, I, and L; V, I, and T. Other conservative amino acid substitutions are also recognized as valid, depending on the context of the amino acid in question. For example, in some cases, methionine (M) can substitute for lysine (K). In addition, sequences that differ by conservative variations are generally homologous.

According to some embodiments, the term “homology” or “homologous” refers to identity of two or more nucleic acid sequences; or identity of two or more amino acid sequences; or the identity of an amino acid sequence to one or more nucleic acid sequence. According to some embodiments, the homology is a global homology, e.g., a homology over the entire amino acid or nucleic acid sequences of the invention and not over portions thereof. The degree of homology or identity between two or more sequences can be determined using various known sequence comparison tools which are described in WO2014/102774.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct or an expression construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

An expression construct can permit transcription of a particular polynucleotide sequence in a host cell (e.g., a prokaryotic cell or a eukaryotic cell). An expression cassette may be part of a plasmid, viral genome, or nucleic acid fragment. Typically, an expression cassette includes a polynucleotide to be transcribed, operably linked to a promoter. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, the term “operably linked” refers to polynucleotide sequences or amino acid sequences placed into a functional relationship with one another. For example, regulatory sequences (e.g., a promoter or enhancer) are “operably linked” to a polynucleotide (e.g., encoding a guide RNA or nucleic acid-guided nuclease) if the regulatory sequences regulate or contribute to the modulation of the transcription or translation of the polynucleotide. Similarly, two polypeptide-encoding nucleotide sequences are operably linked if they are contiguous and capable of expression in the same reading frame so as to produce a “fusion protein” following transcription and translation.

As used herein, the terms “nuclease” and “endonuclease” are used interchangeably to refer to naturally-occurring or engineered enzymes, which cleave a phosphodiester bond within a polynucleotide chain.

As used herein, the term “isolated” refers to being at least partially separated from the natural environment. For example, an isolated cell can refer to a cell that is at least partially separated from its natural environment, e.g., from a plant or animal.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “conjugation” or “conjugated” as used herein, refers to the physical or chemical complexation formed between a first molecule (e.g., a protein of interest (POI)) and a second molecule (e.g., an ssDNA). The chemical complexation constitutes specifically a bond or chemical moiety formed between a functional group of the first molecule with a functional group of the second molecule. For example, in a POI-ssDNA conjugate of the present disclosure, a bond may be formed between a DNA-binding protein (DBP), which is fused to the N- or C-terminal of the POI, and its corresponding ssDNA recognition sequence in the ssDNA. Such bonds include, but are not limited to, covalent linkages and non-covalent bonds, while such chemical moieties include, but are not limited to, esters, carbonates, imines phosphate esters, hydrazones, acetals, orthoesters, peptide linkages, and oligonucleotide linkages. Conjugation can also be achieved via a physical association or non-covalent complexation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

### Polypeptide-ssDNA Conjugate

Described herein are polypeptide-ssDNA conjugates. Polypeptide-ssDNA conjugates are conjugates comprising a polypeptide and a single-stranded DNA (ssDNA). Polypeptide-ssDNA conjugates are generated by intracellular conjugation between a polypeptide and an ssDNA, when the polypeptide and the ssDNA are co-expressed in the same compartment (e.g., in a cell). For example, a POI-ssDNA conjugate can be generated by intracellular conjugation between a polypeptide of interest (POI) and an ssDNA, when the POI and the ssDNA are co-expressed in a cell. The cell can be a eukaryotic cell (e.g., yeast cell (e.g., Saccharomyces cerevisiae), worm cell (e.g., Caenorhabditis elegans cell), insect cell, mammalian cell, etc.), a prokaryotic cell (e.g., Escherichia coli), or an artificial cell. The polypeptide (e.g., POI) can be conjugated to the ssDNA by a covalent bond or a non-covalent bond. The bond can form between a DNA-binding protein (DBP), which is fused to the N- or C-terminal of the polypeptide, and its corresponding ssDNA recognition sequence. In particular, a POI-ssDNA conjugate of the present disclosure can comprise a POI fused to a DBP, an ssDNA recognition sequence corresponding to the DBP (i.e., recognized by the DBP), and a unique ssDNA barcode corresponding to the POI (i.e., with information indicative of the identity of the POI). Also provided here are compositions comprising a POI-ssDNA conjugate. In certain embodiments, the composition is a pharmaceutical composition, e.g., a composition comprising a POI-ssDNA conjugate and a pharmaceutically acceptable carrier.

In certain instances, a POI-ssDNA conjugate can refer to a conjugate that comprises a fusion protein and an ssDNA, wherein the fusion protein comprises: (a) a POI; and (b) a DBP that is fused to the N- or C-terminal of the POI. Accordingly, a POI-ssDNA conjugate can be generated by intracellular conjugation between a fusion protein and an ssDNA, when the fusion protein and the ssDNA are co-expressed in the same compartment in a cell.

### Fusion Protein

A fusion protein of the present disclosure can comprise a polypeptide of interest (POI) and a DBP (e.g., a DBP that is fused to the POI). In some instances, a fusion protein of the present disclosure further comprises a purification tag (e.g., FLAG, His-tag, biotin tag, etc.). In some embodiments, a linker is a present between the POI and the DBP. Linkers are described, for example, in Argos, Mol Biol 211:943-958, 1990; George and Heringa, Protein Eng 15:871-879, 2002; Chen et al., Biotechniques 49:513-518, 2010; and Chen et al., Adv Drug Deliv Rev 65 (10): 1357-1369, 2013. A linker for use in a fusion protein described herein can be a flexible linker, a rigid linker, and/or an in vivo cleavable linker.

For example, a linker for use in a fusion protein described herein can be a flexible linker. Flexible linkers are usually applied when the protein domains that need to be joined require a certain degree of movement or interaction. They are generally composed of small, non-polar (e.g., Gly) or polar (e.g., Ser or Thr) amino acids. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduce the unfavorable interaction between the linker and the protein moieties. An example of the most widely used flexible linker is the sequence (Gly-Gly-Gly-Gly-Ser)n (SEQ ID NO: 35).

Additionally, or in the alternative, a linker for use in a fusion protein described herein can be a rigid linker. While flexible linkers have the advantage of connecting the functional domains passively and permitting a certain degree of movement, the lack of rigidity of these linkers can be a limitation. There are several examples in the literature where the use of flexible linkers resulted in poor expression yields or loss of biological activity. Under such situations, rigid linkers can be successfully applied to keep a fixed distance between the domains and to maintain their independent functions. Rigid linkers exhibit relatively stiff structures by adopting a-helical conformations or by containing multiple Pro residues. Examples of some rigid linkers are: (EAAAK)n (SEQ ID NO: 36) and (XP)n, with X designating any amino acid, preferably Ala, Lys, or Glu.

Additionally, or in the alternative, a linker for use in a fusion protein described herein can be an in vivo cleavable linker. Flexible and rigid linkers represent stable linkers that covalently join functional protein domains together to act as one molecule throughout the in vivo processes that the component protein(s) are involved in. This stable linkage between functional domains provides many advantages such as a prolonged plasma half-life (e.g., albumin or Fc-fusions). However, it also has several potential drawbacks, including, steric hindrance between functional domains, decreased bioactivity, and altered biodistribution and metabolism of the protein moieties due to the interference between domains. Under such circumstances, cleavable linkers are used to release free functional domains in vivo. This type of linker may reduce steric hindrance, improve bioactivity, or achieve independent actions/metabolism of individual domains of recombinant fusion proteins after linker cleavage. The design of in vivo cleavable linkers in recombinant fusion proteins is quite challenging. Unlike the versatility of crosslinking agents available for chemical conjugation methods, linkers in recombinant fusion proteins must necessarily be oligopeptides. For example, an in vivo cleavable disulfide linker (LEAGCKNFFPRISFTSCGSLE) (SEQ ID NO: 37), based on the reversible nature of the disulfide bond, was designed for recombinant fusion proteins by Chen et al. (Biotechniques 49:513-518, 2010), and offered the advantage of generating a precisely constructed, homogeneous product by recombinant methods.

### Protein of Interest (POI)

A protein of interest (POI) can be any protein that is capable of being conjugated to an ssDNA in accordance with the methods described herein. For example, in some embodiments, the POI is a cell penetrating peptide (CPP). In some embodiments, the POI is a ligand, or portion thereof. In other embodiments, the POI is an antigen-binding protein. In some embodiments, the antigen binding protein is a nanobody, a domain antibody, an scFv, a Fab, a diabody, a BiTE, a diabody, a DART, a minibody, a F(ab′)₂, an intrabody, or an antibody mimetic. In certain embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, or a versabody, an aptamer, or a cyclotide.

A POI can be natural, recombinant, or synthetic. In some embodiments, the POI is one selected from a library of POIs. In some embodiments, the POI can be selected from a library of randomly mutated proteins. Accordingly, in some embodiments, the method can include mutagenizing a POI (e.g., through random mutagenesis) and preparing a library of mutagenized proteins. The mutagenized POIs can then be assessed as candidate delivery vehicles and/or candidate therapeutic, as described herein.

In some embodiments, a POI is a protein or peptide found in a protein or peptide database (for example, SWISS-PROT, TrEMBL, SBASE, PFAM, CPPsite, or others known in the art), or a fragment or variant thereof. A POI may be a protein or peptide that may be derived (for example, by transcription and/or translation) from a nucleic acid sequence known in the art, such as a nucleic acid sequence found in a nucleic acid database (for example, GenBank, TIGR, CPPsite, or others known in the art), or a fragment or variant thereof.

Thus, a POI can be any polypeptide, such as, without limitation, peptides, proteins, enzymes, hormones, transporters, nanobodies, single-chain variable fragments (scFv), antigen-binding fragments (Fab), and antibodies or fragments thereof. In certain instances, a POI is a bio-therapeutic, such as a biologic. For example, a POI can be, without limitation, a hormone (e.g., a steroid hormone, such as estrogen, testosterone, etc.), a vaccine, an antitoxin (e.g., an anti-venom), a recombinant protein (e.g., insulin, erythropoietin, a cytokine, etc.), an interleukin, or an antibody (e.g., a monoclonal antibody) or a fragment thereof (e.g., an antigen-binding fragment (Fab)). A POI can be expressed from a variety of different constructs or vectors, such as, without limitation, linear inserts, circular plasmids, or chromosomally integrated DNA. A POI can be expressed in a wide variety of cells, such as, without limitation, in a eukaryotic cell (e.g., yeast cell (e.g., Saccharomyces cerevisiae), worm cell (e.g., Caenorhabditis elegans cell), insect cell, mammalian cell, etc.), a prokaryotic cell (e.g., Escherichia coli), or an artificial cell.

The POI can be a full-length protein, a peptide fragment, or a protein or peptide comprised within a complex. In some instances, a POI is obtained by fragmenting a protein or peptide. The fragmenting step can include fragmenting the protein or peptide with trypsin, Lys-C, another fragmentation enzyme, alternative protein fragmentation or degradation methods, or combinations thereof.

### DNA Binding Protein

In a fusion protein, a DBP can be fused to the N-terminal of a POI. Alternatively, the DBP can be fused to the C-terminal of the POI. In a POI-ssDNA conjugate of the present disclosure, a fusion protein can be conjugated to an ssDNA by a bond (e.g., a covalent bond or a non-covalent bond) between a DBP (e.g., a DBP that is fused to the POI) and an ssDNA recognition sequence corresponding to that DBP. In certain instances, the DBP is a HUH-endonuclease. HUH-endonucleases are endonucleases with a conserved histidine-hydrophobic-histidine (HUH) motif. Representative DBPs and their corresponding recognition sequences are listed in Table 1 below. In certain instances, a DBP of the present disclosure is a HUH endonuclease described in Table 1.

For example, a DBP of the present disclosure can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 1. The ssDNA recognition sequence of the DBP can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 8. In particular instances, the DBP is PCV (porcine circovirus 2), which is fused to N- or C-terminal of a POI of the present disclosure. In such instances, in a POI-ssDNA conjugate of the present disclosure, the POI is conjugated to an ssDNA by a bond (e.g., a covalent bond or a non-covalent bond), wherein the bond is formed between PCV (SEQ ID NO: 1) and the ssDNA recognition sequence of PCV (SEQ ID NO: 8). PCV is described in literature, e.g., in Vega-Rocha et al., 2007.

Alternatively, a DBP of the present disclosure can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 2. The ssDNA recognition sequence of the DBP can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 9. In particular instances, the DBP is DCV (duck circovirus), which is fused to N- or C-terminal of a POI of the present disclosure. In such instances, in a POI-ssDNA conjugate of the present disclosure, the POI is conjugated to an ssDNA by a bond (e.g., a covalent bond or a non-covalent bond), wherein the bond is formed between DCV (SEQ ID NO: 2) and the ssDNA recognition sequence of DCV (SEQ ID NO: 9). DCV is described in literature, e.g., in Hu et al., 2019.

Alternatively, a DBP of the present disclosure can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 3. The ssDNA recognition sequence of the DBP can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 10. In particular instances, the DBP is VirD2 (e.g., relaxase protein VirD2 of Agrobacterium tumefaciens), which is fused to N- or C-terminal of a POI of the present disclosure. In such instances, in a POI-ssDNA conjugate of the present disclosure, the POI is conjugated to an ssDNA by a bond (e.g., a covalent bond or a non-covalent bond), wherein the bond is formed between VirD2 (SEQ ID NO: 3) and the ssDNA recognition sequence of VirD2 (SEQ ID NO: 10). VirD2 is described in literature, e.g., in Bernardinelli et al., 2017.

Alternatively, a DBP of the present disclosure can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 4. The ssDNA recognition sequence of the DBP can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 11. In particular instances, the DBP is RepB (e.g., replication protein RepB Streptococcus agalactiae), which is fused to N- or C-terminal of a POI of the present disclosure. In such instances, in a POI-ssDNA conjugate of the present disclosure, the POI is conjugated to an ssDNA by a bond (e.g., a covalent bond or a non-covalent bond), wherein the bond is formed between RepB (SEQ ID NO: 4) and the ssDNA recognition sequence of RepB (SEQ ID NO: 11). RepB is described in literature, e.g., in Boer et al., 2009.

Alternatively, a DBP of the present disclosure can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 5. The ssDNA recognition sequence of the DBP can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 12. In particular instances, the DBP is TraI (e.g., conjugation protein TraI of Escherichia coli), which is fused to N- or C-terminal of a POI of the present disclosure. In such instances, in a POI-ssDNA conjugate of the present disclosure, the POI is conjugated to an ssDNA by a bond (e.g., a covalent bond or a non-covalent bond), wherein the bond is formed between TraI (SEQ ID NO: 5) and the ssDNA recognition sequence of TraI (SEQ ID NO: 12). TraI is described in literature, e.g., in Datta et al., 2003.

Alternatively, a DBP of the present disclosure can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 6. The ssDNA recognition sequence of the DBP can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 13. In particular instances, the DBP is mMobA (e.g., mobilization protein A of Escherichia coli), which is fused to N- or C-terminal of a POI of the present disclosure. In such instances, in a POI-ssDNA conjugate of the present disclosure, the POI is conjugated to an ssDNA by a bond (e.g., a covalent bond or a non-covalent bond), wherein the bond is formed between mMobA (SEQ ID NO: 6) and the ssDNA recognition sequence of mMobA (SEQ ID NO: 13). mMobA is described in literature, e.g., in Monzingo et al., 2007.

Alternatively, a DBP of the present disclosure can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 7. The ssDNA recognition sequence of the DBP can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 14. In particular instances, the DBP is NES (e.g., nicking enzyme of Staphylococcus aureus), which is fused to N- or C-terminal of a POI of the present disclosure. In such instances, in a POI-ssDNA conjugate of the present disclosure, the POI is conjugated to an ssDNA by a bond (e.g., a covalent bond or a non-covalent bond), wherein the bond is formed between NES (SEQ ID NO: 7) and the ssDNA recognition sequence of NES (SEQ ID NO: 14). NES is described in literature, e.g., in Edwards et al., 2013.

TABLE 1
Representative HUH endonucleases and corresponding recognition
sequences

| HUH | | Recognition |
|---|---|---|
| endonuclease | HUH sequence | sequence |
| PCV | MPSKKNGRSGPQPHKRWVFTLNNPSEDERKKIR | aagtattaccagaaa |
| | DLPISLFDYFIVGEEGNEEGRTPHLQGFANFVKK | (SEQ ID NO: 8) |
| | QTFNKVKWYLGARCHIEKAKGTDQQNKEYCSK | |
| | EGNLLMECGAPRSQGQR | |
| | (SEQ ID NO: 1) | |
| DCV | MAKSGNYSYKRWVFTINNPTFEDYVHVLEFCTL | aagtattaccag |
| | DNCKFAIVGEEKGANGTPHLQGFLNLRSNARAA | (SEQ ID NO: 9) |
| | ALEESLGGRAWLSRARGSDEDNEEYCAKESTYL | |
| | RVGEPVSKGRSSDLAEATSAV | |
| | (SEQ ID NO: 2) | |
| VirD2 | MPDRAQVIIRIVPGGGTKTLQQIINQLEYLSRKGK | caacggtatatatcctgc |
| | LELQRSARHLDIPVPPDQIRELAQSWVTEAGIYDE | ca |
| | SQSDDDRQQDLTTHIIVSFPAGTDQTAAYEASRE | (SEQ ID NO: 10) |
| | WAAEMFGSGYGGGRYNYLTAYHVDRDHPHLH | |
| | VVVNRRELLGHGWLKISRRHPQLNYDGLRKKM | |
| | AEISLRHGIVLDATSRAERGIAERPITYAEHRRLE | |
| | RMQPW | |
| | (SEQ ID NO: 3) | |
| RepB | MAKEKARYFTFLLYPESIPSDWELKLETLGVPMA | tgcttccgtactacgacc |
| | ISPLHDKDKSSIKGQKYKKAHYHVLYIAKNPVTA | cccca |
| | DSVRKKIKLLLGEKSLAMVQVVLNVENMYLYLT | (SEQ ID NO: 11) |
| | HESKDAIAKKKHVYDKADIKLINNFDIDRY | |
| | (SEQ ID NO: 4) | |
| TraI | MMSIAQVRSAGSAGNYYTDKDNYYVLGSMGER | tttgcgtggggtgtggtg |
| | WAGRGAEQLGLQGSVDKDVFTRLLEGRLPDGA | cttt |
| | DLSRMQDGSNRHRPGYDLTFSAPKSVSMMAML | (SEQ ID NO: 12) |
| | GGDKRLIDAHNQAVDFAVRQVEALASTRVMTD | |
| | GQSETVLTGNLVMALFNHDTSRDQEPQLHTHAV | |
| | VANVTQHNGEWKTLSSDKVGKTGFIENVYANQI | |
| | AFGRLYREKLKEQVEALGYETEVVGKHGMWEM | |
| | PGVPVEAFSGRSQTIREAVGEDASLKSRDVAALD | |
| | TRKSKQHVDPEIKMAEWMQTLKETGFDIRAYRD | |
| | AADQRADLRTLTPGPASQDGPDVQQAVTQAIAG | |
| | LSER | |
| | (SEQ ID NO: 5) | |
| mMobA | MAIYHLTAKTGSRSGGQSARAKADYIQREGKYA | ccagtttctcgaagagaa |
| | RDMDEVLHAESGHMPEFVERPADYWDAADLYE | accggtaagtgcaccct |
| | RANGRLFKEVEFALPVELTLDQQKALASEFAQH | ccc |
| | LTGAERLPYTLAIHAGGGENPHCHLMISERINDGI | (SEQ ID NO: 13) |
| | ERPAAQWFKRYNGKTPEKGGAQKTEALKPKAW | |
| | LEQTREAWADHANRALERAGH | |
| | (SEQ ID NO: 6) | |
| NES | MAMYHFQNKFVSKANGQSATAKSAYNSASRIK | acgcgaacggaacgttc |
| | DFKENEFKDYSNKQCDYSEILLPNNADDKFKDR | gcataagtgcgcccttac |
| | EYLWNKVHDVENRKNSQVAREIIIGLPNEFDPNS | gggatttaac |
| | NIELAKEFAESLSNEGMIVDLNIHKINEENPHAHL | (SEQ ID NO: 14) |
| | LCTLRGLDKNNEFEPKRKGNDYIRDWNTKEKHN | |
| | EWRKRWENVQNKHLEKNGFSVRVSADSYKNQN | |
| | IDLEPTKKEGWKARKFEDETG | |
| | (SEQ ID NO: 7) | |

In some embodiments, a fusion protein of the present disclosure (e.g., a fusion protein comprising a POI variant, a DBP (e.g., a DBP that is fused to the N- or C-terminal of the POI), and/or a purification tag) can comprise an antigen-binding domain from an antibody, e.g., a Fab comprising a heavy chain variable (VH) domain and/or a light chain variable (VL) domain, e.g., from an IgG. A VH domain of a fusion protein can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 27. Additionally, or in the alternative, a VL domain of a fusion protein can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 28. In certain instances, a fusion protein of the present disclosure comprises the VH domain of TRV-A-001, or a VH domain that comprises an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. Additionally, or in the alternative, a fusion protein of the present disclosure can comprise the VL domain of TRV-A-001, or a VL domain that comprises an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. In particular, a fusion protein of the present disclosure can be TRV-A-001 and/or can comprise an anti-human transferrin receptor 1 Fab fused to PCV.

Alternatively, a VH domain of a fusion protein can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 29. Additionally, or in the alternative, a VL domain of a fusion protein can comprise an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 30. In certain instances, a fusion protein of the present disclosure comprises the VH domain of TRV-A-002, or a VH domain that comprises an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. Additionally, or in the alternative, a fusion protein of the present disclosure can comprise the VL domain of TRV-A-002, or a VL domain that comprises an amino acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. In particular, a fusion protein of the present disclosure can be TRV-A-002 and/or can comprise an anti-mouse transferrin receptor 1 Fab fused to PCV.

### Nucleic Acids

Also disclosed herein are nucleic acid sequences encoding a fusion protein, such as nucleic acid sequences encoding a POI, a DBP, and/or a purification tag. The present disclosure further provides plasmids or expression constructs for fusion proteins, such as, plasmids or expression constructs that comprise nucleic acid sequences encoding a fusion protein.

In some instances, an expression construct for a fusion protein comprises a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 33. In some instances, an expression construct for a fusion protein comprises a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to one or more of nucleotides 6-116, nucleotides 178-229, and/or nucleotides 237-1199 of SEQ ID NO: 33. In some instances, an expression construct for a fusion protein comprises a nucleic acid sequence comprising at least 5 nucleotides (e.g., about 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more nucleotides) from one or more of nucleotides 6-116, nucleotides 178-229, and/or nucleotides 237-1199 of SEQ ID NO: 33. For example, the expression construct can comprise a nucleic acid sequence comprising one or more of nucleotides 6-116, nucleotides 178-229, and/or nucleotides 237-1199 of SEQ ID NO: 33. In certain instances, an expression construct for a fusion protein comprises the nucleic acid sequence of TRV-R-001, or a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. In particular, an expression construct for a fusion protein can be TRV-R-001.

In some instances, an expression construct for a fusion protein comprises a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 34. In certain instances, an expression construct for a fusion protein comprises the nucleic acid sequence of TRV-R-002, or a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. In particular, an expression construct for a fusion protein can be TRV-R-002.

In some instances, an expression construct for a fusion protein comprises a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 38. In some instances, an expression construct for a fusion protein comprises a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to one or more of nucleotides 2-113, nucleotides 175-227, and/or nucleotides 234-1196 of SEQ ID NO: 38. In some instances, an expression construct for a fusion protein comprises a nucleic acid sequence comprising at least 5 nucleotides (e.g., about 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more nucleotides) from one or more of nucleotides 2-113, nucleotides 175-227, and/or nucleotides 234-1196 of SEQ ID NO: 38. For example, the expression construct can comprise a nucleic acid sequence comprising one or more of nucleotides 2-113, nucleotides 175-227, and/or nucleotides 234-1196 of SEQ ID NO: 38. In certain instances, an expression construct for a fusion protein comprises the nucleic acid sequence of TRV-R-003, or a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. In particular, an expression construct for a fusion protein can be TRV-R-003.

Single-Stranded DNA (ssDNA)

An ssDNA of the present disclosure comprises a recognition sequence corresponding to a DBP, such as a DBP described in Table 1. For example, a ssDNA recognition sequence of the present disclosure can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 8, 9, 10, 11, 12, 13, or 14. In some instances, an ssDNA comprises a recognition sequence corresponding to the DBP with which the ssDNA is co-expressed in a cell.

An expression construct described herein can comprise a nucleic acid sequence encoding an ssDNA. The nucleic acid sequence encoding the ssDNA can further comprise a non-coding RNA (ncRNA). In some instances, the nucleic acid sequence encoding the ssDNA is inserted into the ncRNA. In some instances, the ncRNA comprises a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to nucleotides 6-116 and nucleotides 178-229 of SEQ ID NO: 33. For example, the ncRNA can comprise two parts, wherein a first part comprises a nucleic acid sequence having at least 80% sequence identity to nucleotides 6-116 of SEQ ID NO: 33, and a second part comprises a nucleic acid sequence having at least 80% sequence identity to nucleotides 178-229 of SEQ ID NO: 33. In some instances, the ncRNA comprises at least 5 nucleotides (e.g., about 5, 10, 15, 20, 25, 30, 35, 40, 45, 50, 55, 60, 65, 70, 75, 80, 85, 90, 95, 100, or more nucleotides) from nucleotides 6-116 and nucleotides 178-229 of SEQ ID NO: 33. For example, the ncRNA can comprise two parts, wherein a first part comprises at least 5 nucleotides from nucleotides 6-116 of SEQ ID NO: 33, and a second part comprises at least 5 nucleotides from nucleotides 178-229 of SEQ ID NO: 33. In some instances, the ncRNA comprises nucleotides 6-116 and nucleotides 178-229 of SEQ ID NO: 33. For example, the ncRNA can comprise two parts, wherein a first part comprises nucleotides 6-116 of SEQ ID NO: 33, and a second part comprises nucleotides 178-229 of SEQ ID NO: 33. The nucleic acid sequence encoding the ssDNA can be inserted into the ncRNA between the first and second parts.

Additionally, or in the alternative, an ncRNA described herein can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to nucleotides 2-113 and nucleotides 175-227 of SEQ ID NO: 38. For example, the ncRNA can comprise two parts, wherein a first part comprises a nucleic acid sequence having at least 80% sequence identity to nucleotides 2-113 of SEQ ID NO: 38, and a second part comprises a nucleic acid sequence having at least 80% sequence identity to nucleotides 175-227 of SEQ ID NO: 38. In some instances, the ncRNA comprises at least 5 nucleotides (e.g., about 5, 10, 15, 20, 25, 30, 35, 40, 45, 50, 55, 60, 65, 70, 75, 80, 85, 90, 95, 100, or more nucleotides) from nucleotides 2-113 and nucleotides 175-227 of SEQ ID NO: 38. For example, the ncRNA can comprise two parts, wherein a first part comprises at least 5 nucleotides from nucleotides 2-113 of SEQ ID NO: 38, and a second part comprises at least 5 nucleotides from nucleotides 175-227 of SEQ ID NO: 38. In some instances, the ncRNA comprises nucleotides 2-113 and nucleotides 175-227 of SEQ ID NO: 38. For example, the ncRNA can comprise two parts, wherein a first part comprises nucleotides 2-113 of SEQ ID NO: 38, and a second part comprises nucleotides 175-227 of SEQ ID NO: 38. The nucleic acid sequence encoding the ssDNA can be inserted into the ncRNA between the first and second parts.

In certain instances, the nucleic acid sequence encoding the ssDNA is transcribed into an ssDNA product (e.g., an ssDNA product that contains the ssDNA recognition sequence and a unique ssDNA barcode). In some instances, the ncRNA comprises a stem loop structure, and sequence encoding the ssDNA is inserted into the stem loop structure. Stem loop structures are known in the art (see, e.g., Forsdyke, Journal of Theoretical Biology (1998), 192:489-504; Broude, Trends Biotechnol (2002) 20:249-256).

Expression constructs described here can further comprise a sequence encoding a reverse transcriptase (RT), such as an RT that is compatible with the ncRNA. In some instances, the expression construct comprises the sequence encoding the RT at the 5′ end of the ncRNA. In some instances, the expression construct comprises the sequence encoding the RT at the 3′ end of the ncRNA. The sequence encoding the RT can have at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to nucleotides 237-1199 of SEQ ID NO: 33 or nucleotides 234-1196 of SEQ ID NO: 38. In some instances, the sequence encoding the RT comprises at least 5 nucleotides (e.g., about 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more nucleotides) from nucleotides 237-1199 of SEQ ID NO: 33 or nucleotides 234-1196 of SEQ ID NO: 38. For example, the sequence encoding the RT can comprise nucleotides 237-1199 of SEQ ID NO: 33 or nucleotides 234-1196 of SEQ ID NO: 38.

The ncRNA and/or RT described herein can be derived from Retron-Eco1 (Ec86), Retron-Eco2 (Ec67), Retron-Eco3 (Ec73), Retron-Eco4 (Ec83), Retron-Eco5 (Ec107), Retron-Eco6 (Ec48), Retron-Eco7 (Ec78), Retron-Mxa1 (Mx162), Retron-Mxa2 (Mx65), Retron-Sau1 (Sa163), Retron-Nex1 (Ne160), Retron-Nex2 (Ne144), Retron-Sen1 (Se72), Retron-Sen2 (St85), Retron-Vch1 (Vc95), Retron-Vch2 (Vc81), Retron-Vch3 (Vc137), Retron-Vpa1 (Vp96), Retron-Kpn1, Retron-Pmi1, Retron-Rxx1, Retron-Bxx1, Retron-Fel1, Retron-Cco1, Retron-Adi1, Retron-Age1, Retron-Cfe1, Retron-Cfu1, Retron-Cvi1, Retron-Mli1, Retron-Mco1, Retron-Mfu1, Retron-Mma1, Retron-Mst1, Retron-Mvi1, Retron-Cap1, Retron-Cpe1, or Retron-Sce1.

An ssDNA of the present disclosure can also comprise a barcode (e.g., an ssDNA barcode). As used herein, a “barcode” refers to any nucleic acid sequence with information indicative of at least one molecule's identity, i.e., a nucleic acid sequence that can any nucleic acid sequence that can uniquely identify at least one molecule. For example, an ssDNA of the present disclosure can comprise a barcode with information indicative of the identity of a particular POI. A barcode (e.g., an ssDNA barcode) may be generated from a variety of different formats, including bulk synthesized polynucleotide barcodes, randomly synthesized barcode sequences, microarray based barcode synthesis, native nucleotides, a partial complement with an N-mer, a random N-mer, a pseudo random N-mer, or combinations thereof. In some embodiments, the barcode can be a non-naturally occurring sequence. The barcode (e.g., ssDNA barcode) can comprise, for example, about 5 to about 400 nucleotides, such as about 10 to about 300 nucleotides, about 15 to about 200 nucleotides, or about 20 to about 100 nucleotides (e.g., about 5, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nucleotides), or more than 400 nucleotides. In certain embodiments, the barcode (e.g., ssDNA barcode) comprises about 20 to about 100 nucleotides, such as about 30 to about 90 nucleotides, about 40 to about 80 nucleotides, or about 50 to about 70 nucleotides (e.g., about 20, 25, 30, 35, 40, 45, 50, 55, 60, 65, 70, 75, 80, 85, 90, 95, or 100 nucleotides). Further, the barcode can be located anywhere on or adjacent to the ssDNA recognition sequence.

The barcode (e.g., ssDNA barcode) may also include additional sequence segments. Such additional sequence segments may include functional sequences, such as primer sequences, primer annealing site sequences, immobilization sequences, or other recognition or binding sequences useful for subsequent processing, e.g., a sequencing primer or primer binding site for use in sequencing of samples to which the ssDNA barcode is attached.

Representative ssDNA barcodes for use in the method of the present disclosure are listed in Table 2 below. In certain instances, an ssDNA barcode of the present disclosure is an ssDNA barcode described in Table 2.

For example, an ssDNA barcode of the present disclosure can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 15. In particular instances, the barcode is TRV-O-001 and/or comprises the nucleic acid sequence set forth in SEQ ID NO: 15.

Alternatively, an ssDNA barcode of the present disclosure can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 16. In particular instances, the barcode is TRV-O-002 and/or comprises the nucleic acid sequence set forth in SEQ ID NO: 16.

Alternatively, an ssDNA barcode of the present disclosure can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 17. In particular instances, the barcode is TRV-O-003 and/or comprises the nucleic acid sequence set forth in SEQ ID NO: 17.

Alternatively, an ssDNA barcode of the present disclosure can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 18. In particular instances, the barcode is TRV-O-004 and/or comprises the nucleic acid sequence set forth in SEQ ID NO: 18.

Alternatively, an ssDNA barcode of the present disclosure can comprise a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 19. In particular instances, the barcode is TRV-O-005 and/or comprises the nucleic acid sequence set forth in SEQ ID NO: 19.

TABLE 2
Representative ssDNA barcodes

| ssDNA | | Sequence |
|---|---|---|
| barcode | Sequence | Identifier |
| TRV-O-001 | AAGTATTACCAGACGTGTGCTCTTCCGATCTGAGGGT | SEQ ID NO: 15 |
| | ACTTAGATCGGAAGAGCGTCG*T*G*T-biotin | |
| TRV-O-002 | AAGTATTACCAGACGTGTGCTCTTCCGATCTGACAAT | SEQ ID NO: 16 |
| | TGCCAGATCGGAAGAGCGTCG*T*G*T-biotin | |
| TRV-O-003 | AAGTATTACCAGACGTGTGCTCTTCCGATCTTAACGC | SEQ ID NO: 17 |
| | ACCTAGATCGGAAGAGCGTCG*T*G*T-biotin | |
| TRV-O-004 | AAGTATTACCAGACGTGTGCTCTTCCGATCTATGATC | SEQ ID NO: 18 |
| | GTCGAGATCGGAAGAGCGTCG*T*G*T-biotin | |
| TRV-O-005 | AAGTATTACCAGACGTGTGCTCTTCCGATCTTGTCTC | SEQ ID NO: 19 |
| | CCATAGATCGGAAGAGCGTCG*T*G*T-biotin | |

PCV recognition sequence: in bold + italics; Universal binding site where forward primer (SEQ ID NO: 25) binds: in italics; Variable region or ′barcode sequence′: in bold; Universal binding site 2 where reverse primer (SEQ ID NO: 24) binds: not bold, not italics

Also disclosed herein are plasmids or expression constructs for ssDNA, such as plasmids or expression constructs that comprise nucleic acid sequences encoding an ssDNA, such as DNA sequences encoding for an ssDNA recognition sequence and an ssDNA barcode.

In some instances, an expression construct for an ssDNA comprises a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 32. In certain instances, an expression construct for an ssDNA comprises the nucleic acid sequence of SUMO-PCV, or a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. In particular, an expression construct for an ssDNA can be SUMO-PCV.

An ssDNA described herein can be expressed from a variety of different constructs or vectors, such as, without limitation, linear inserts, circular plasmids, or chromosomally integrated DNA. There are many methods known in the literature to generate ssDNA intracellularly, which include, without limitation, single-stranded phagemids (Praetorius et al., Nature 552:84, 2017), rolling circle replication (Hao et al., Cells 9:467, 2020), reversed transcription of non-coding RNA (ncRNA) (Chen et al., Gene Ther 10:1776, 2003; Li et al., Oligonucleotides 20 (2): 61-68, 2010; Elbaz et al., Nat Commun 7:11179, 2016; Alon et al., Acs Synth Biol 9:236, 2020), and retron systems (Farzadfard et al., Science 346 (6211): 1256272, 2014; Simon et al., Nucleic Acids Res 47:11007, 2019; Lopez et al., Nat Chem Biol, 2021; Kong et al., Protein Cell 12:899, 2021).

### Expressing POI-ssDNA Conjugates in Cells

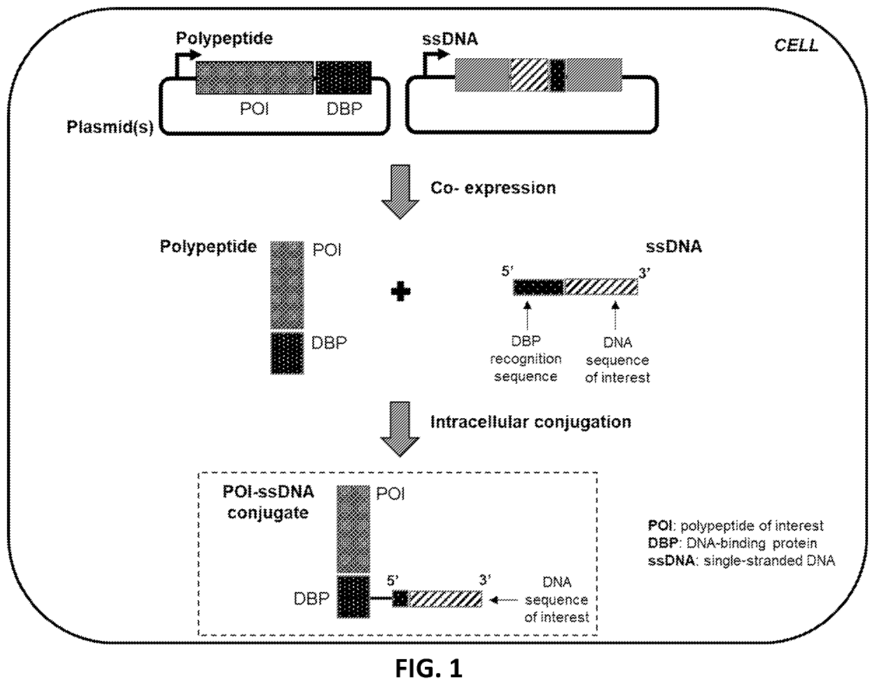

Also disclosed herein are methods for generating POI-ssDNA conjugates, such as methods for expressing POI-ssDNA conjugates in cells. A schematic representation of methods for expressing POI-ssDNA conjugates in cells is provided in FIG. 1.

A POI-ssDNA conjugate can be generated (e.g., expressed in a cell), for example, by transforming a cell with an expression vector (e.g., a plasmid) comprising: (i) a nucleic acid sequence (e.g., DNA sequence) encoding for a fusion protein, e.g., a fusion protein comprising a POI variant and a DBP (e.g., a DBP that is fused to the N- or C-terminal of the POI); (ii) a nucleic acid sequence (e.g., DNA sequence) encoding for the corresponding ssDNA, such as a nucleic acid sequence encoding for an ssDNA recognition sequence corresponding to the DBP, and a unique ssDNA barcode corresponding to the POI variant (e.g., barcode that can be used to identify the POI variant); and (iii) one or more promoters to drive expression of the fusion protein (e.g., fusion protein comprising the POI variant and the DBP) and the ssDNA. The expression vector (e.g., plasmid) can comprise a single promoter to drive expression of the fusion protein (e.g., fusion protein comprising POI variant, DBP, and/or purification tag) and the ssDNA. Alternatively, the expression vector can comprise two separate promoters, one for the fusion protein and one for the ssDNA. The expression vector can further comprise a nucleic acid sequence encoding for a purification tag. For example, the expression vector (e.g., plasmid) may encode a fusion protein that comprises a POI, a DBP (e.g., a DBP that is fused to the N- or C-terminal of the POI), and a purification tag.

Alternatively, a POI-ssDNA conjugate can be generated (e.g., expressed in a cell) by transforming a cell with two expression vector (e.g., plasmids), such as a first expression vector and a second expression vector. In such instances, one expression vector (e.g., a first expression vector) may comprise: (i) a nucleic acid sequence (e.g., DNA sequence) encoding for a fusion protein, e.g., a fusion protein comprising a POI variant and a DBP (e.g., a DBP that is fused to the N- or C-terminal of the POI); and (ii) a promoter to drive the expression of the fusion protein. This expression vector (e.g., the first expression vector) may further comprise a nucleic acid sequence encoding for a purification tag. For example, the first expression vector may encode a fusion protein that comprises a POI, a DBP (e.g., a DBP that is fused to the POI), and a purification tag. The other expression vector (e.g., a second expression vector) may comprise: (i) a nucleic acid sequence (e.g., DNA sequence) encoding for the corresponding ssDNA, such as a nucleic acid sequence encoding for an ssDNA recognition sequence corresponding to the DBP (e.g., DBP that is encoded by the first expression vector), and a unique ssDNA barcode corresponding to the POI variant (e.g., POI variant that is encoded by the first expression vector); and (ii) a promoter to drive the expression of the ssDNA. The unique ssDNA barcode encoded by the second expression vector can be used to identify the POI variant encoded by the first expression vector, when a POI-ssDNA conjugate is generated by co-expression of the first expression vector and the second expression vector in a single compartment (e.g., in a cell).

Promoters for use in the compositions and methods of the present disclosure may include, without limitation, T7, T7lac, lac, Sp6, araBAD, trp, Ptac, pL, T3CMV, SV40, EF1a, PGK1, Ubc, human beta actin, CAG, TRE, UAS, Ac5, POlyhedrin, CaMKIIa, GAL-1,10, TEF1, GDS, ADH1, CaMV35S, H1, U6, or any other promoters suitable for the host organism.

The present method allows a plurality of (e.g., a pool of) POI-ssDNA conjugates comprising a plurality of different POI variants to be made, each with a unique barcode, i.e., a unique barcode with information indicative of the identity of the POI variant. A plurality of (e.g., a pool of) POI-ssDNA conjugates can comprise about 10² to about 10¹⁴ (e.g., about 10³ to about 10¹⁴, about 10⁴ to about 10¹⁴, about 10⁵ to about 10¹⁴, about 10⁶ to about 10¹⁴, about 10⁷ to about 10¹⁴, about 10⁸ to about 10¹⁴, about 10⁹ to about 10¹⁴, about 10¹⁰ to about 10¹⁴, about 10¹¹ to about 10¹⁴, about 10¹² to about 10¹⁴, about 10¹³ to about 10¹⁴, about 10² to about 10¹³, about 10³ to about 10¹³, about 10⁴ to about 10¹³, about 10⁵ to about 10¹³, about 10⁶ to about 10¹³, about 10⁷ to about 10¹³, about 10⁸ to about 10¹³, about 10⁹ to about 10¹³, about 10¹⁰ to about 10¹³, about 10¹¹ to about 10¹³, about 10¹² to about 10¹³, about 10² to about 10¹², about 10³ to about 10¹², about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10² to about 10¹¹, about 10³ to about 10¹¹, about 10⁴ to about 10¹¹, about 10⁵ to about 10¹¹, about 10⁶ to about 10¹¹, about 10⁷ to about 10¹¹, about 10⁸ to about 10¹¹, about 10⁹ to about 10¹¹, about 10¹⁰ to about 10¹¹, about 10² to about 10¹⁰, about 10³ to about 10¹⁰, about 10⁴ to about 10¹⁰ about 10⁵ to about 10¹⁰, about 10⁶ to about 10¹⁰, about 10⁷ to about 10¹⁰, about 10⁸ to about 10¹⁰, about 10⁹ to about 10¹⁰, about 10² to about 10⁹, about 10³ to about 10⁹, about 10⁴ to about 10⁹, about 10⁵ to about 10⁹, about 10⁶ to about 10⁹, about 10⁷ to about 10⁹, or about 10⁸ to about 10⁹) POI variants. Co-expression of POI variants with their respective unique barcodes in a single compartment (e.g., in a cell) enables simultaneous screening and identification of those POI variants amongst a pool of POI variants.

A polypeptide (e.g., a POI) that is conjugated to a unique ssDNA barcode with which the polypeptide can be screened and/or identified (e.g., by using methods of the present disclosure), is referred to herein as a DNA encoded polypeptide (DEP). In some instances, a DEP refers to a POI-ssDNA conjugate, wherein a POI is conjugated to its unique ssDNA barcode with which the POI can be screened and/or identified by using the methods described herein.

Also provided here are compositions comprising a DEP of the present disclosure. In certain embodiments, the composition is a pharmaceutical composition, e.g., a composition comprising a DEP and a pharmaceutically acceptable carrier.

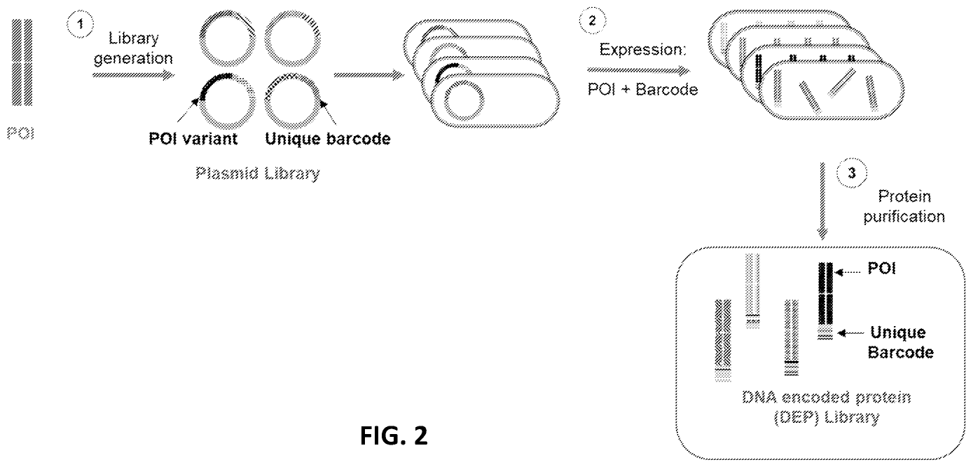

The present disclosure provides methods for generating a library of DEPs. A DEP library can contain about 10² to about 10¹⁴ (e.g., about 10³ to about 10¹⁴, about 10⁴ to about 10¹⁴, about 10⁵ to about 10¹⁴, about 10⁶ to about 10¹⁴, about 10⁷ to about 10¹⁴, about 10⁸ to about 10¹⁴, about 10⁹ to about 10¹⁴, about 10¹⁰ to about 10¹⁴, about 10¹¹ to about 10¹⁴, about 10¹² to about 10¹⁴, about 10¹³ to about 10¹⁴, about 10² to about 10¹³, about 10³ to about 10¹³, about 10⁴ to about 10¹³, about 10⁵ to about 10¹³, about 10⁶ to about 10¹³, about 10⁷ to about 10¹³, about 10⁸ to about 10¹³, about 10⁹ to about 10¹³, about 10¹⁰ to about 10¹³, about 10¹¹ to about 10¹³, about 10¹² to about 10¹³, about 10² to about 10¹², about 10³ to about 10¹², about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10² to about 10¹¹, about 10³ to about 10¹¹, about 10⁴ to about 10¹¹, about 10⁵ to about 10¹¹, about 10⁶ to about 10¹¹, about 10⁷ to about 10¹¹, about 10⁸ to about 10¹¹, about 10⁹ to about 10¹¹, about 10¹⁰ to about 10¹¹, about 10² to about 10¹⁰, about 10³ to about 10¹⁰, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁹ to about 10¹⁰, about 10² to about 10⁹, about 10³ to about 10⁹, about 10⁴ to about 10⁹, about 10⁵ to about 10⁹, about 10⁶ to about 10⁹, about 10⁷ to about 10⁹, or about 10⁸ to about 10⁹) DEP variants. A schematic representation of methods for generating a DEP library is provided in FIG. 2.

A DEP library can be generated from a pool of expression vectors (e.g., a pool of plasmids). A pool of expression vectors can comprise about 10² to about 10¹⁴ (e.g., about 10³ to about 10¹⁴, about 10⁴ to about 10¹⁴, about 10⁵ to about 10¹⁴, about 10⁶ to about 10¹⁴, about 10⁷ to about 10¹⁴, about 10⁸ to about 10¹⁴, about 10⁹ to about 10¹⁴, about 10¹⁰ to about 10¹⁴, about 10¹¹ to about 10¹⁴, about 10¹² to about 10¹⁴, about 10¹³ to about 10¹⁴, about 10² to about 10¹³, about 10³ to about 10¹³, about 10⁴ to about 10¹³, about 10⁵ to about 10¹³, about 10⁶ to about 10¹³, about 10⁷ to about 10¹³, about 10⁸ to about 10¹³, about 10⁹ to about 10¹³, about 10¹⁰ to about 10¹³, about 10¹¹ to about 10¹³, about 10¹² to about 10¹³, about 10² to about 10¹², about 10³ to about 10¹², about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10² to about 10¹¹, about 10³ to about 10¹¹, about 10⁴ to about 10¹¹, about 10⁵ to about 10¹¹, about 10⁶ to about 10¹¹, about 10⁷ to about 10¹¹, about 10⁸ to about 10¹¹, about 10⁹ to about 10¹¹, about 10¹⁰ to about 10¹¹, about 10² to about 10¹⁰, about 10³ to about 10¹⁰, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁹ to about 10¹⁰, about 10² to about 10⁹, about 10³ to about 10⁹, about 10⁴ to about 10⁹, about 10⁵ to about 10⁹, about 10⁶ to about 10⁹, about 10⁷ to about 10⁹, or about 10⁸ to about 10⁹) expression vectors. Each expression vector (e.g., plasmid) can contain, without limitation: (i) a nucleic acid sequence (e.g., DNA sequence) encoding for a fusion protein, e.g., a fusion protein comprising a POI variant and a DBP (e.g., a DBP that is fused to the N- or C-terminal of the POI); (ii) a nucleic acid sequence (e.g., DNA sequence) encoding for the corresponding ssDNA, such as a nucleic acid sequence encoding for ssDNA recognition sequence corresponding to the DBP and unique ssDNA barcode corresponding to the POI variant (e.g., barcode that can be used to identify the POI variant); and (iii) one or more promoters to drive expression of the fusion protein and the ssDNA.

In some instances, an expression vector (e.g., plasmid) described herein further encodes a purification tag. For example, an expression vector of the present disclosure may encode a fusion protein that comprises a POI, a DBP (e.g., a DBP that is fused to the N- or C-terminal of the POI), and a purification tag.

In some instances, an expression vector (e.g., plasmid) described herein comprises DNA sequence encoding for a non-coding RNA (ncRNA). The ncRNA can be recognized by its compatible reverse transcriptase (RT) and transcribed into an ssDNA product that contains the ssDNA recognition sequence and the unique ssDNA barcode. The RT can be a retron reverse transcriptase, a human immunodeficiency virus type 1 reverse transcriptase, a moloney murine leukemia virus reverse transcriptase, and others. The RT can be included in the same expression vector, or a different expression vector, or can be constitutively expressed in the host organism. When a retron is employed to express the ssDNA, the ncRNA and RT pair can be derived from Retron-Eco1 (Ec86), Retron-Eco2 (Ec67), Retron-Eco3 (Ec73), Retron-Eco4 (Ec83), Retron-Eco5 (Ec107), Retron-Eco6 (Ec48), Retron-Eco7 (Ec78), Retron-Mxa1 (Mx162), Retron-Mxa2 (Mx65), Retron-Sau1 (Sa163), Retron-Nex1 (Ne160), Retron-Nex2 (Ne144), Retron-Sen1 (Se72), Retron-Sen2 (St85), Retron-Vch1 (Vc95), Retron-Vch2 (Vc81), Retron-Vch3 (Vc137), Retron-Vpa1 (Vp96), Retron-Kpn1, Retron-Pmi1, Retron-Rxx1, Retron-Bxx1, Retron-Fel1, Retron-Cco1, Retron-Adi1, Retron-Age1, Retron-Cfe1, Retron-Cfu1, Retron-Cvi1, Retron-Mli1, Retron-Mco1, Retron-Mfu1, Retron-Mma1, Retron-Mst1, Retron-Mvi1, Retron-Cap1, Retron-Cpe1, or Retron-Sce1.

In some instances, an expression vector (e.g., plasmid) described hereinabove comprises one promoter to drive expression of the fusion protein (e.g., fusion protein comprising POI variant, DBP, and/or purification tag) and the ssDNA. Alternatively, the expression vector can comprise two separate promoters, one for the fusion protein and one for the ssDNA. Promoters for use in the compositions and methods of the present disclosure include, without limitation, T7, T7lac, lac, Sp6, araBAD, trp, Ptac, pL, T3CMV, SV40, EF1a, PGK1, Ubc, human beta actin, CAG, TRE, UAS, Ac5, POlyhedrin, CaMKIIa, GAL-1,10, TEF1, GDS, ADH1, CaMV35S, H1, U6, or any other promoters suitable for the host organism.

Expression vectors (e.g., a pool of expression vectors) can be transformed into cells at a dilution such that there is about one expression vector (e.g., plasmid) per cell. In some instances, expression vectors are transformed into cells at a dilution such that at least one (e.g., 1, 2, 3, 4, 5, 6, 7, 8, 9, 10, 11, 12, 13, 14, 15, 16, 17, 18, 19, 20, or more) expression vector is introduced into each cell. Each cell can express a variant of the POI, which is covalently linked via a DBP intracellularly to a unique ssDNA barcode. The unique ssDNA barcode can be used to identify the variant in subsequent experiments. The pool of DEPs can be obtained from a pool of expression vectors for subsequent in vitro or in vivo experiments by any conventional methods of protein expression and purification.

In some instances, a DEP can be generated by:
(i) transforming an expression construct or expression vector (e.g., a plasmid) into cells under conditions in which about one (e.g., at least one (e.g., 1, 2, 3, 4, 5, 6, 7, 8, 9, 10, 11, 12, 13, 14, 15, 16, 17, 18, 19, 20, or more)) expression vector is introduced into each cell, wherein the expression vector comprises nucleic acids encoding (a) a fusion protein (e.g., fusion protein comprising a POI variant, a DBP that is fused to the POI, and/or a purification tag); and (b) ssDNA comprising ssDNA recognition sequence that is recognized by the DBP and a unique nucleic acid barcode corresponding to the POI; and
(ii) culturing the cells under conditions in which the expression vector is expressed, and the DBP portion of the fusion protein binds to its corresponding ssDNA recognition sequence, thereby producing a DEP.

In some instances, the method further includes purifying the DEPs. The DEPs can be purified using any number of methods, resulting in only conjugates containing both the POI fusion protein and the corresponding ssDNA barcode to be collected. Simply by way of example, the POI-ssDNA conjugates can be pulled down from a cell lysate via a purification tag (e.g., FLAG, His-tag, biotin-tag, etc.), which can be included in the fusion protein. The POI-ssDNA conjugates can then be washed and released from the anti-His beads or streptavidin-beads or other pull-down assays compatible with the purification tag used, and further purified using a streptavidin-coated bead and a biotinylated oligo that is complementary to a sequence in the ssDNA barcode. After this pull-down step, a mixture of beads are obtained that are bound to the POI-ssDNA conjugate, biotinylated oligonucleotides annealed to random DNA sequences, or nothing. The POI-ssDNA conjugate can be released from the streptavidin-coated beads and purified by heating and washing the mixture to denature the DNA and biotinylated oligonucleotide or by releasing the complex using restriction endonucleases. In some embodiments, the method further includes eluting the DEP from the beads by using gentle elution buffers such as glycine to release the POI without denaturing the POI-ssDNA binding.

In some embodiments, the method involves producing a plurality (e.g., a library) of expression vectors, as described hereinabove. In some embodiments, the method involves producing a plurality (e.g., a library) of DEPs, as outlined above.

The plurality of expression vectors or DEPs may be a library of expression vectors or DEPs. The term “library” refers to a mixture of heterogeneous polypeptides or nucleic acids. The library is composed of members, which have a single polypeptide or nucleic acid sequence. Sequence differences, between library members, such as sequence differences between different POIs, or POI-ssDNA conjugates, or DEPs, or expression vectors are responsible for the diversity present in the library. The library may take the form of a simple mixture of polypeptides or nucleic acids, or may be in the form organisms or cells, for example bacteria, viruses, animal or plant cells and the like, transformed with a library of nucleic acids, such as expression vectors of the present disclosure. Preferably, each individual organism or cell contains only one member of the library.

Expression vectors can be assembled from DNA encoding components of interest (e.g., a POI, a fusion protein, and/or an ssDNA). The DNA can be obtained from any source, such as through amplification of sequences of interest from genomic DNA or through synthesis. DNA encoding a component of interest (e.g., a POI, a fusion protein, and/or an ssDNA) can be amplified and cloned using a known technique, such as PCR using appropriately-selected primers, in order to produce sufficient quantities of the DNA and to modify the DNA in such a manner (e.g., by addition of appropriate restriction sites) that it can be introduced as an insert into an expression vector. Amplified and cloned DNA can be further diversified, using mutagenesis, such as PCR, in order to produce a greater diversity or wider repertoire of POIs, as well as novel POIs.

A cloned polynucleotide encoding any vector component described herein (e.g., a POI, a fusion protein, and/or an ssDNA) is introduced into an expression vector (e.g., a plasmid), such as vectors described herein. In the case of polynucleotides encoding proteins or fusion proteins, the polynucleotide is inserted into the vector in such a manner that the protein will be expressed as protein in appropriate host cells.

In some embodiments, the method further comprises sequencing one or more portions of the vector. For example, the method may further include sequencing one or more portions of the vector encoding the ssDNA and/or the POI, thereby establishing an association between the POI and the ssDNA barcode. This association can be used to provide a reference or index for identifying the POI based on the presence of the ssDNA barcode, for example, at later steps in the method. Such identification can be accomplished by sequencing, e.g., next generation sequencing. Sequencing can be performed using automated Sanger sequencing (ABI 3730x1 genome analyzer), pyrosequencing on a solid support (454 sequencing, Roche), sequencing-by-synthesis with reversible terminations (ILLUMINA® Genome Analyzer), sequencing-by-ligation (ABI SOLID®) or sequencing-by-synthesis with virtual terminators (HELISCOPE®); Moleculo sequencing (see Voskoboynik et al. eLife 2013 2: e00569 and U.S. patent application Ser. No. 13/608,778, filed Sep. 10, 2012); DNA nanoball sequencing; Single molecule real time (SMRT) sequencing; Nanopore DNA sequencing; Sequencing by hybridization; Sequencing with mass spectrometry; and Microfluidic Sanger sequencing. Exemplary next generation sequencing methods known to those of skill in the art include Massively parallel signature sequencing (MPSS), Polony sequencing, pyrosequencing (454), Illumina (Solexa) sequencing by synthesis, SOLID sequencing by ligation, Ion semiconductor sequencing (Ion Torrent sequencing), DNA nanoball sequencing, chain termination sequencing (Sanger sequencing), Heliscope single molecule sequencing, Single molecule real time (SMRT) sequencing (Pacific Biosciences) and nanopore sequencing such as is described at world wide website nanoporetech.com.

These libraries of vectors are then introduced in host cells, which can be eukaryotic or prokaryotic, for expression of one or more components encoded on the vector (e.g., a POI, a fusion protein, and/or an ssDNA). Transfer of the vector into host cells (e.g., by infection, transformation, or transfection) can be carried out using known techniques, such as electroporation, protoplast fusion, or calcium phosphate co-precipitation. In cases where the method requires two vectors, both libraries can be introduced into appropriate host cells either simultaneously or sequentially.

### Compartmentalized Expression of POI-ssDNA Conjugates

In some embodiments, the method further involves introducing the expression vector into a host cell suitable to express the POI-ssDNA conjugate, and expressing the POI-ssDNA conjugate in the host cell, such that expressed POI-ssDNA conjugate each comprises a POI and the corresponding ssDNA barcode. In some embodiments, the expression vector is in a plurality of expression vectors and the plurality of expression vectors is transferred into host cells under conditions such that the average expression vector per host cell is 1 or more (e.g., 1, 2, 3, 4, 5, 6, 7, 8, 9, 10, 11, 12, 13, 14, 15, 16, 17, 18, 19, 20, or more). In some embodiments, the vector is in a plurality of expression vectors and the plurality of expression vectors are transferred into host cells under conditions such that the average expression vector per host cell is less than 1. The POI-ssDNA conjugate can be expressed from the expression vector in the host cell, wherein the expressed POI-ssDNA conjugate is encoded on the vector and comprises a POI and its corresponding ssDNA barcode.

Also provided herein are cells, e.g., host cells, comprising expression vectors of the present disclosure. For example, provided herein are isolated cells (e.g., isolated host cells) comprising expression vectors, wherein the expression vectors comprises: (i) a nucleic acid sequence (e.g., DNA sequence) encoding for a fusion protein, e.g., a fusion protein comprising a POI variant and a DBP (e.g., a DBP that is fused to the N- or C-terminal of the POI); (ii) a nucleic acid sequence (e.g., DNA sequence) encoding for the corresponding ssDNA, such as a nucleic acid sequence encoding for an ssDNA recognition sequence corresponding to the DBP, and a unique ssDNA barcode corresponding to the POI variant (e.g., barcode that can be used to identify the POI variant); and (iii) one or more promoters to drive expression of the fusion protein (e.g., fusion protein comprising the POI variant and the DBP) and the SSDNA.

The term “host cell” refers to a cell that can express proteins, protein fragments, or peptides of interest from a vector. For example, the host cell may be a prokaryotic cell (e.g., a bacterial cell) or a eukaryotic cell (e.g., a yeast cell (e.g., a S. cerevisiae cell, Pichia pastoris, or the like), a plant cell, a fungal cell, an insect cell, a mammalian cell, etc.). In some instances, the bacterial cell is an E. coli cell.

In some embodiments, the host cell is a mammalian cultured cell derived from rodents (rats, mice, guinea pigs, or hamsters) such as CHO, BHK, NSO, SP2/0, YB2/0; or human tissues or hybridoma cells, yeast cells, or insect cells. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.” In certain embodiments, the mammalian cell is a COP cell, an L cell, a C127 cell, an Sp2/0 cell, an NS-0 cell, an NIH3T3 cell, a PC12 cell, a PC12h cell, a BHK cell, a CHO cell, a COS1 cell, a COS3 cell, a COST cell, a CVI cell, a Vero cell, a HeLa cell, an HEK-293 cell, a PER C6 cell, a cell derived from diploid fibroblasts, a myeloma cell, or HepG2.

Methods of introducing polynucleotides (e.g., an expression vector) into host cells are known in the art and are typically selected based on the kind of host cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

Alternatively, the method may involve transferring the expression vector to a non-cellular compartment (e.g., an emulsion droplet) suitable to express the POI-ssDNA conjugate, and expressing the POI-ssDNA conjugate in the non-cellular compartment (e.g., the emulsion droplet), such that POI-ssDNA conjugates, each comprising a POI and its corresponding ssDNA barcode, are formed.

In certain embodiments, the non-cellular compartment is a droplet, such as a droplet in an emulsion and/or a microfluidic droplet. Emulsification can be used in the methods of the disclosure to separate or segregate a sample or set of samples into a series of compartments, for example a compartment having a single cell or a discrete portion of an acellular sample, such as a cell-free extract or a cell-free transcription and/or cell-free translation mixture. Typically, as used in conjunction with the methods and compositions disclosed herein, an emulsion will include a plurality of droplets, each droplet including a vector, such that each droplet includes a vector encoding one test agent and ssDNA barcode that distinguishes it from the other droplets. Emulsification can be used in the methods of the disclosure to compartmentalize one or more target molecules in emulsion droplets with one vector encoding an ssDNA barcode. Droplets in an emulsion can be sorted and/or isolated according to methods well known in the art. For example, double emulsion droplets containing a fluorescence signal can be analyzed and/or sorted using conventional fluorescence-activated cell sorting (FACS) machines at rates of >104 droplets s″1, and have been used to improve the activity of enzymes produced by single cells or by in vitro translation of single genes (Aharoni et al., Chem Biol 12 (12): 1281-1289, 2005; Mastrobattista et al., Chem Biol 2 (12): 1291-1300, 2005). However, the emulsions are highly polydisperse, limiting quantitative analysis, and it is difficult to add new reagents to pre-formed droplets (Griffiths et al., Trends Biotechnol 24 (9): 395-402, 2006). These limitations can, however, be overcome by using protocols based on droplet-based microfluidic systems (see for example Teh et al., Lab on a chip 8 (2): 198-220, 2008; Theberge et al., Angew Chem Int Ed Engl 49 (34): 5846-5868, 2010; and Guo et al., Lab on a chip 12 (12): 2146, 2012) in which highly monodisperse droplets of picoliter volume can be made (Anna et al., Appl Phys Lett 82 (3): 364-366, 2003), fused (Song et al., Angew Chem Int Edit 42 (7): 767-772, 2003; Chabert et al., Electrophoresis 26 (19): 3706-3715, 2005), split (Song et al., Angew Chem Int Edit 42 (7): 767-772, 2003; Link et al., Phys Rev Lett 92 (5): 054503, 2004), incubated (Song et al., Angew Chem Int Edit 42 (7): 767-772, 2003; Frenz et al., Lab on a chip 9 (10): 1344-1348, 2009), and sorted triggered on fluorescence (Baret, et al, Lab on a chip 9 (13): 1850-1858, 2009), at kHz frequencies, such as those described in Mazutis et al. (Nat. Protoc. 8 (5): 870-891, 2013), incorporated by reference herein. As disclosed herein, an emulsion can include various compounds, enzymes, or reagents in addition to the target molecules, target nucleic acids and origin-specific barcodes. These additives may be included in the emulsion solution prior to emulsification. Alternatively, the additives may be added to individual droplets after emulsification.

Emulsion may be achieved by a variety of methods known in the art (see, for example, US 2006/0078888 A1, of which paragraphs [0139]-[0143] are incorporated by reference herein). An exemplary emulsion is a water-in-oil emulsion. In some embodiments, the continuous phase of the emulsion includes a fluorinated oil. An emulsion can contain a surfactant or emulsifier (for example, a detergent, anionic surfactant, cationic surfactant, or amphoteric surfactant) to stabilize the emulsion. Other oil/surfactant mixtures, for example, silicone oils, may also be utilized in particular embodiments. An emulsion can be contained in a well or a plurality of wells, such as a plate, for easy of handling. In some examples, one or more vector molecules, target nucleic acid and nucleic acid barcodes (e.g., ssDNA barcodes) are compartmentalized. An emulsion can be a monodisperse emulsion or a polydisperse emulsion. In certain embodiments, the droplet may contain an acellular system, such as a cell-free extract. The emulsion in context with the present disclosure may include various compounds, enzymes, or reagents in addition to the vector to achieve cell-free transcription or translation. These additives may be included in the emulsion solution prior to emulsification. Alternatively, the additives may be added to individual droplets after emulsification.

### Isolation of POI-ssDNA Conjugates

In some embodiments, the method further involves isolating the POI-ssDNA conjugates from a host cell comprising an expression vector described herein. Any purification methods can be used to isolate nucleoproteins from a host cell. Exemplary isolation techniques include, without limitation, affinity capture, immunoprecipitation, chromatography (for example, size exclusion chromatography, hydrophobic interaction chromatography, reverse-phase chromatography, ion exchange chromatography, affinity chromatography, metal binding chromatography, immunoaffinity chromatography, high performance liquid chromatography (HPLC), and liquid chromatography-mass spectrometry (LC-MS)), electrophoresis, hybridization to a capture oligonucleotide, phenol-chloroform extraction, minicolumn purification, or ethanol or isopropanol precipitation. Chromatography methods are described in detail, for example, in Hedhammar et al. (“Chromatographic methods for protein purification,” Royal Institute of Technology, Stockholm, Sweden), which is incorporated herein by reference. Such techniques can utilize a capture molecule that recognizes a labeled POI-ssDNA conjugate, or a fusion protein or ssDNA associated with the POI-ssDNA conjugate.

Testing ssDNA Barcodes

Isolated POI-ssDNA conjugates, comprising a POI and a unique identifying ssDNA barcode, can be assessed for cell targeting capacity and/or for suitability as a biotherapeutic by contacting (e.g., co-incubating) the POI-ssDNA conjugate with a target cell. For example, the contacting step may involve incubating, exposing, or mixing cells with the POI-ssDNA conjugate. The cells can be in any conditions or cell media suitable for cell viability. Further, the cells may be attached to a surface or suspended in cell media. After contacting POI-ssDNA conjugates with a target cell, nucleic acids inside the target cell can then be assessed to identify internalized ssDNA barcode. In some embodiments, the method involves isolating the nucleic acids from the target cell, or a fraction thereof. For example, in some embodiments, the isolated nucleic acid is obtained from cytoplasm that is extracted from the target cell prior to nucleic acid isolation. Alternatively, the isolated nucleic acid is obtained from membrane-bound organelles (e.g., nucleus, endoplasmic reticulum, Golgi apparatus, vacuole, lysosome, or mitochondria) that are extracted from the target cell prior to nucleic acid isolation.

The nucleic acids obtained from a target cell following contact with a test POI-ssDNA conjugate (e.g., a test DEP) can be amplified for further analysis following any amplification methods known in the art. An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); realtime reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others. In certain embodiments, the testing step comprises reverse-transcribing the isolated RNA to producing cDNA, and sequencing the cDNA to determine the presence of the ssDNA barcode sequence. In some embodiments, the testing step comprises sequencing the isolated RNA to determine the presence of the ssDNA barcode sequence.

Other exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self-sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6, 124,090 and 5,612, 199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art.

The nucleic acid (e.g., isolated nucleic acids) obtained can be tested for the presence of the ssDNA barcode sequence by a variety of methods, including any sequencing or microarray methods known in the art. In some embodiments, the identity of a unique identifying nucleic acid is determined by DNA or RNA sequencing. For example, the sequencing can be performed using automated Sanger sequencing (ABI 3730x1 genome analyzer), pyrosequencing on a solid support (454 sequencing, Roche), sequencing-by-synthesis with reversible terminations (ILLUMINA® Genome Analyzer), sequencing-by-ligation (ABI SOLID®) or sequencing-by-synthesis with virtual terminators (HELISCOPE®); Moleculo sequencing (see Voskoboynik et al. eLife 2013 2: e00569 and U.S. patent application Ser. No. 13/608,778, filed Sep. 10, 2012); DNA nanoball sequencing; Single molecule real time (SMRT) sequencing; Nanopore DNA sequencing; Sequencing by hybridization; Sequencing with mass spectrometry; and Microfluidic Sanger sequencing. Exemplary next generating sequencing methods known to those of skill in the art include Massively parallel signature sequencing (MPSS), Polony sequencing, pyrosequencing (454), Illumina (Solexa) sequencing by synthesis, SOLID sequencing by ligation, Ion semiconductor sequencing (Ion Torrent sequencing), DNA nanoball sequencing, chain termination sequencing (Sanger sequencing), Heliscope single molecule sequencing, Single molecule real time (SMRT) sequencing (Pacific Biosciences) and nanopore sequencing such as is described at world wide website nanoporetech.com.

The presence of the ssDNA barcode sequence can indicate that an associated POI is suitable for use a cell targeting agent (e.g., a delivery vehicle) or a biotherapeutic. For example, identification of the POI as a candidate delivery vehicle may be based on a previously established reference or index. In some embodiments, the candidate delivery vehicle identified by the present methods is a protein that targets a biotherapeutic into a compartment of the target cell or binds to the cell surface of the target cell. For example, the delivery vehicle can be suitable for targeting a biotherapeutic to a membrane-bound organelle or cytoplasm. In certain embodiments, the membrane-bound organelle is a nucleus, endoplasmic reticulum, Golgi apparatus, vacuole, lysosome, or mitochondria. In specific embodiments, internalization refers to at least 0.01%, at least 0.05%, at least 0.1%, at least 0.5%, at least 1%, at least 2%, at least 5% at least 10%, at least 15%, or at least 20% of the POI internalized or localized into the cytoplasm of a cell (e.g., within 1 hr, 2 hrs, 3 hrs, 4 hrs, or more after contact of the cell with the POI-ssDNA conjugate).

### Expression Vectors

Also provided herein are expression vectors. Expression vectors may also be referred to herein as expression constructs. Expression vectors may comprise: (i) a nucleic acid sequence (e.g., DNA sequence) encoding for a fusion protein, e.g., a fusion protein comprising a POI variant and a DBP (e.g., a DBP that is fused to the N- or C-terminal of the POI); (ii) a nucleic acid sequence (e.g., DNA sequence) encoding for the corresponding ssDNA, such as a nucleic acid sequence encoding for an ssDNA recognition sequence corresponding to the DBP, and a unique ssDNA barcode corresponding to the POI variant (e.g., barcode that can be used to identify the POI variant); and (iii) one or more promoters to drive expression of the fusion protein (e.g., fusion protein comprising the POI variant and the DBP) and the ssDNA.

In some embodiments, provided herein are two expression vector, such as a first expression vector and a second expression vector. In such instances, one expression vector (e.g., a first expression vector) may comprise: (i) a nucleic acid sequence (e.g., DNA sequence) encoding for a fusion protein, e.g., a fusion protein comprising a POI variant and a DBP (e.g., a DBP that is fused to the N- or C-terminal of the POI); and (ii) a promoter to drive the expression of the fusion protein. This expression vector (e.g., the first expression vector) may further comprise a nucleic acid sequence encoding for a purification tag. For example, the first expression vector may encode a fusion protein that comprises a POI, a DBP (e.g., a DBP that is fused to the POI), and a purification tag. The other expression vector (e.g., a second expression vector) may comprise: (i) a nucleic acid sequence (e.g., DNA sequence) encoding for the corresponding ssDNA, such as a nucleic acid sequence encoding for an ssDNA recognition sequence corresponding to the DBP (e.g., DBP that is encoded by the first expression vector), and a unique ssDNA barcode corresponding to the POI variant (e.g., POI variant that is encoded by the first expression vector); and (ii) a promoter to drive the expression of the ssDNA. The unique ssDNA barcode encoded by the second expression vector can be used to identify the POI variant encoded by the first expression vector, when a POI-ssDNA conjugate is generated by co-expression of the first expression vector and the second expression vector in a single compartment (e.g., in a cell).

Also provided here are cells (e.g., isolated cells), such as host cells, comprising expression vectors of the present disclosure.

“Expression vector” or “vector”, as used herein, refers to a polynucleotide vehicle that can be used to introduce genetic material into a cell. Vectors can be linear or circular. Vectors useful as expression vectors include plasmids, viral vectors (including phage), and integratable DNA fragments (i.e., fragments that can be integrated into the host genome by homologous recombination). The four major types of vectors are plasmids, viral vectors, cosmids, and artificial chromosomes. Vectors can contain a replication sequence capable of effecting replication of the vector in a suitable host cell (i.e., an origin of replication). Typically, vectors comprise an origin of replication, a multicloning site, and/or a selectable marker. Upon transformation of a suitable host, the vector may replicate and function independently of the host genome or integrate into the host genome. Vector design depends, among other things, on the intended use and host cell for the vector, and the design of a vector of the invention for a particular use and host cell is within the level of skill in the art.

General methods for construction of expression vectors are known in the art. Expression vectors for most host cells are commercially available. There are several commercial software products designed to facilitate selection of appropriate vectors and construction thereof, such as bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transformation and gene expression and methods to easily enable cloning of such polynucleotides.

Expression vectors typically comprise regulatory sequences that are involved in one or more of the following: regulation of transcription, post-transcriptional regulation, and regulation of translation. Expression vectors can be introduced into a wide variety of organisms including bacterial cells, yeast cells, mammalian cells, and plant cells. Vectors typically comprise functional regulatory sequences corresponding to the host cells or organism(s) into which they are being introduced. Further, expression vectors can include polynucleotides encoding protein tags (e.g., poly-His tags, hemagglutinin tags, fluorescent protein tags, bioluminescent tags, nuclear localization tags). The coding sequences for such protein tags can be fused to the coding sequences (e.g., a sequence doing a nucleic acid-guided nuclease).

In some aspects, polynucleotides encoding one or more of the various components of the expression vector are operably linked to a promoter. For example, the operably linked promoter can be an inducible promoter, a repressible promoter, or a constitutive promoter. In some embodiments, the expression vector comprises a first promoter operatively linked to the nucleic acid sequence encoding the fusion protein, and comprises a second promoter operatively linked to the nucleic acid sequence encoding the ssDNA. In certain embodiments, the first and second promoter each comprises an inducible element such that the expression level of the fusion protein and the expression level of the ssDNA can be controlled. In certain embodiments, the first and/or second promoter is T7 or T5. In some embodiments, the first and/or second promoter is a constitutive promoter. Alternatively, an expression vector may comprise a single promoter driving the expression of the fusion protein and the ssDNA.

Vectors can be designed for expression of various components of the described methods in prokaryotic or eukaryotic cells. Alternatively, transcription can be in vitro, for example using T7 promoter regulatory sequences and T7 polymerase. Other RNA polymerase and promoter sequences can be used.

Vectors can be introduced into and propagated in a prokaryote. Prokaryotic vectors are well known in the art. Typically a prokaryotic vector comprises an origin of replication suitable for the target host cell (e.g., oriC derived from E. coli, pUC derived from pBR322, pSC101 derived from Salmonella), 15A origin (derived from p15A) or bacterial artificial chromosomes). Vectors can include a selectable marker. A “selectable marker gene” refers to a gene that upon expression confers a phenotype by which successfully transformed cells carrying the vector can be identified. Selectable marker genes as used herein can confer resistance to a selection agent in cell culture and/or confer a phenotype which is identifiable upon visual inspection. In some embodiments, the selectable marker is a gene that upon expression confers resistance to a selection agent (e.g., a drug, e.g., an antibiotic, such asampicillin, chloramphenicol, gentamicin, and kanamycin). Zeocin™ (Life Technologies, Grand Island, NY) can be used as a selection in bacteria, fungi (including yeast), plants and mammalian cell lines. Accordingly, vectors can be designed that carry only one drug resistance gene for Zeocin for selection work in a number of organisms. In some embodiments, the selectable marker is a gene that upon expression confers an identifiable phenotype. For example, the selectable marker may be a fluorescent marker that confers fluorescence in cells carrying the vector that can be identified visually or by machine, e.g., flow cytometry.

Useful promoters are known for expression of proteins in prokaryotes, for example, T5, T7, Rhamnose (inducible), Arabinose (inducible), and PhoA (inducible). Further, T7 promoters are widely used in vectors that also encode the T7 RNA polymerase. Prokaryotic vectors can also include ribosome binding sites of varying strength, and secretion signals (e.g., mal, see, tat, ompC, and pelB). In addition, vectors can comprise RNA polymerase promoters for the expression of gRNAs. Prokaryotic RNA polymerase transcription termination sequences are also well known (e.g., transcription termination sequences from S. pyogenes). Integrating vectors for stable transformation of prokaryotes are also known in the art (see, e.g., Heap, J. T., et al., “Integration of DNA into bacterial chromosomes from plasmids without a counter-selection marker,” Nucleic Acids Res. (2012) 40: e59).

Expression of proteins in prokaryotes is often carried out in a bacteria, such as Escherichia coli with vectors containing constitutive or inducible promoters directing the expression of the expressed components of the vector (e.g., ssDNA and fusion protein).

A wide variety of RNA polymerase promoters suitable for expression of the various components are available in prokaryotes (see, e.g., Jiang, Y., et al., “Multigene editing in the Escherichia coli genome via the CRISPR-Cas9 system,” Environ Microbiol. (2015) 81:2506-2514); Estrem, S. T., et al., (1999) “Bacterial promoter architecture: subsite structure of UP elements and interactions with the carboxy-terminal domain of the RNA polymerase alpha subunit,” Genes Dev. 15;13 (16): 2134-47).

In some aspects, a vector is a yeast expression vector comprising one or more components of the above-described methods. Examples of vectors for expression in Saccharomyces cerivisae include, but are not limited to, the following: pYepSec1, pMFa, pJRY88, pYES2, and picZ. Methods for gene expression in yeast cells are known in the art (see, e.g., Methods in Enzymology, Volume 194, “Guide to Yeast Genetics and Molecular and Cell Biology, Part A,” (2004) Christine Guthrie and Gerald R. Fink (eds.), Elsevier Academic Press, San Diego, CA). Typically, expression of protein-encoding genes in yeast requires a promoter operably linked to a coding region of interest plus a transcriptional terminator. Various yeast promoters can be used to construct expression cassettes for expression of genes in yeast. Examples of promoters include, but are not limited to, promoters of genes encoding the following yeast proteins: alcohol dehydrogenase 1 (ADH1) or alcohol dehydrogenase 2 (ADH2), phosphoglycerate kinase (PGK), triose phosphate isomerase (TPI), glyceraldehyde-3-phosphate dehydrogenase (GAPDH; also known as TDH3, or triose phosphate dehydrogenase), galactose-1-phosphate uridyl-transferase (GAL7), UDP-galactose epimerase (GAL10), cytochrome ci (CYC1), acid phosphatase (PHO5) and glycerol-3-phosphate dehydrogenase gene (GPD1). Hybrid promoters, such as the ADH2/GAPDH, CYC1/GAL10 and the ADH2/GAPDH promoter (which is induced at low cellular-glucose concentrations, e.g., about 0.1 percent to about 0.2 percent) also may be used. In S. pombe, suitable promoters include the thiamine-repressed nmtl promoter and the constitutive cytomegalovirus promoter in pTL2M.

Yeast RNA polymerase III promoters (e.g., promoters from 5S, U6 or RPR1 genes) as well as polymerase III termination sequences are known in the art (see, e.g., yeastgenome.org; Harismendy, O., et al., (2003) “Genome-wide location of yeast RNA polymerase III transcription machinery,” The EMBO Journal. 22 (18): 4738-4747.)

In addition to a promoter, several upstream activation sequences (UASs), also called enhancers, may be used to enhance polypeptide expression. Exemplary upstream activation sequences for expression in yeast include the UASs of genes encoding these proteins: CYC1, ADH2, GAL1, GAL7, GAL10, and ADH2. Exemplary transcription termination sequences for expression in yeast include the termination sequences of the a-factor, CYC1, GAPDH, and PGK genes. One or multiple termination sequences can be used.

Suitable promoters, terminators, and coding regions may be cloned into E. coli-yeast shuttle vectors and transformed into yeast cells. These vectors allow strain propagation in both yeast and E. coli strains. Typically, the vector contains a selectable marker and sequences enabling autonomous replication or chromosomal integration in each host. Examples of plasmids typically used in yeast are the shuttle vectors pRS423, pRS424, pRS425, and pRS426 (American Type Culture Collection, Manassas, VA). These plasmids contain a yeast 2 micron origin of replication, an E. coli replication origin (e.g., pMB1), and a selectable marker.

The various components can also be expressed in insects or insect cells. Suitable expression control sequences for use in such cells are well known in the art. In some aspects, it is desirable that the expression control sequence comprises a constitutive promoter. Examples of suitable strong promoters include, but are not limited to, the following: the baculovirus promoters for the piO, polyhedrin (polh), p 6.9, capsid, UAS (contains a Gal4 binding site), Ac5, cathepsin-like genes, the B. mori actin gene promoter; Drosophila melanogaster hsp70, actin, α-1-tubulin or ubiquitin gene promoters, RSV or MMTV promoters, copia promoter, gypsy promoter, and the cytomegalovirus IE gene promoter. Examples of weak promoters that can be used include, but are not limited to, the following: the baculovirus promoters for the iel, ie2, ieO, etl, 39K (aka pp31), and gp64 genes. If it is desired to increase the amount of gene expression from a weak promoter, enhancer elements, such as the baculovirus enhancer element, hr5, may be used in conjunction with the promoter.

For the expression of some of the components disclosed herein in insects, RNA polymerase III promoters are known in the art, for example, the U6 promoter. Conserved features of RNA polymerase III promoters in insects are also known (see, e.g., Hernandez, G., (2007) “Insect small nuclear RNA gene promoters evolve rapidly yet retain conserved features involved in determining promoter activity and RNA polymerase specificity,” Nucleic Acids Res. 2007 January; 35 (1): 21-34).

In another aspect, the various components are incorporated into mammalian vectors for use in mammalian cells. A large number of mammalian vectors suitable for use with the systems of the present invention are commercially available (e.g., from Life Technologies, Grand Island, NY; NeoBiolab, Cambridge, MA; Promega, Madison, WI; DNA2.0, Menlo Park, CA; Addgene, Cambridge, MA).

Vectors derived from mammalian viruses can also be used for expressing the various components of the present methods in mammalian cells. These include vectors derived from viruses such as adenovirus, papovirus, herpesvirus, polyomavirus, cytomegalovirus, lentivirus, retrovirus, vaccinia and Simian Virus 40 (SV40) (see, e.g., Kaufman, R. J., (2000) “Overview of vector design for mammalian gene expression,” Molecular Biotechnology, Volume 16, Issue 2, pp 151-160; Cooray S., et al., (2012) “Retrovirus and lentivirus vector design and methods of cell conditioning,” Methods Enzymol. 507:29-57). Regulatory sequences operably linked to the components can include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. Commonly used promoters are constitutive mammalian promoters CMV, EF1a, SV40, PGK1 (mouse or human), Ubc, CAG, CaMKIIa, and beta-Act, and others known in the art (Khan, K. H. (2013) “Gene Expression in Mammalian Cells and its Applications,” Advanced Pharmaceutical Bulletin 3 (2), 257-263). Further, mammalian RNA polymerase III promoters, including HI and U6, can be used.

Numerous mammalian cell lines have been utilized for expression of gene products including HEK 293 (Human embryonic kidney) and CHO (Chinese hamster ovary). These cell lines can be transfected by standard methods (e.g., using calcium phosphate or polyethyleneimine (PEI), or electroporation). Other typical mammalian cell lines include, but are not limited to: HeLa, U2OS, 549, HT1080, CAD, P19, NIH 3T3, L929, N2a, Human embryonic kidney 293 cells, MCF-7, Y79, SO-Rb50, Hep G2, DUKX-X11, J558L, and Baby hamster kidney (BHK) cells. In certain embodiments, the mammalian cell is a COP cell, an L cell, a C127 cell, an Sp2/0 cell, an NS-0 cell, an NIH3T3 cell, a PC12 cell, a PC12 h cell, a BHK cell, a CHO cell, a COS1 cell, a COS3 cell, a COST cell, a CVI cell, a Vero cell, a HeLa cell, an HEK-293 cell, a PER C6 cell, a cell derived from diploid fibroblasts, a myeloma cell, or HepG2.

Methods of introducing polynucleotides (e.g., an expression vector) into host cells are known in the art and are typically selected based on the kind of host cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

### Use of DEP Library

Described herein are potential applications of the compositions and methods of the present disclosure. Using the methods described herein, polypeptide variants (e.g., POI variants) can be screened to identify variants useful as delivery vehicles and/or bio-therapeutics. For example, by using DEP libraries generated by the present methods, POI variants can be screened so as to identify POI variants that would be useful as delivery vehicles and/or bio-therapeutics.

Libraries screened using the present methods can comprise a variety of types of polypeptides. A given library can comprise a set of structurally related or unrelated polypeptides. In some instances, the POI variants and libraries thereof can be obtained by systematically altering the structure of a first POI variant, e.g., a first variant that is structurally similar to a known natural binding partner of the target polypeptide, e.g., using methods known in the art or the methods described herein, and correlating that structure to a resulting biological activity (e.g., efficient intracellular uptake, efficient endosomal escape, efficient traverse into the subcellular compartments, longer half-life in plasma or cells or tissues, etc.), e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a polypeptide variant. For example, in one embodiment, a general library of polypeptides is screened using the methods described herein (e.g., using a DEP library wherein each DEP comprises a particular POI variant).

In some embodiments, a DEP comprising a particular POI variant is applied to a test sample, e.g., a cell or living tissue or organ (e.g., cell or tissue from pancreas, liver, kidney, eye, etc.), and one or more effects of the POI variant is evaluated. For example, a composition comprising a DEP can be applied to test sample. In certain embodiments, the composition is a pharmaceutical composition, e.g., a comprising a DEP and a pharmaceutically acceptable carrier. In a cultured or primary cell, for example, the POI variant can be tested for efficient intracellular uptake, efficient endosomal escape, efficient traverse into the subcellular compartments, longer half-life, etc. In some embodiments, the test sample is, or is derived from (e.g., a sample taken from) an in vivo model of a disease or disorder. The in vivo model can be a model for a disease or disorder, which can be treated and/or managed with a POI variant that is screened by the present methods and identified to be useful as a bio-therapeutic. As an in vivo model, an animal model can be used. The animal can be a mouse, rat, Guinea pig, or other rodent. The animal can also be from a higher non-human species, including, but not limited to a non-human primate, mini-pig, sheep, dog, etc.

Methods for evaluating each of these effects are known in the art. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289 (5485): 1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used.

A POI variant that has been screened by a method described herein and determined to have efficient intracellular uptake, efficient endosomal escape, efficient traverse into the subcellular compartments, longer half-life, etc. can be considered a candidate POI variant. A candidate POI variant that has been screened, e.g., in an in vivo model of a disorder (e.g., Type 1 and Type 2 diabetes mellitus, cancer, etc.), and determined to have a desirable effect on the disorder, e.g., on one or more symptoms of the disorder, can be considered a candidate therapeutic agent. Once the candidate therapeutic agent is screened in a clinical setting, it can be considered a therapeutic agent. Candidate POI variants, candidate therapeutic agents, and therapeutic agents can be optionally optimized and/or derivatized, and formulated with physiologically acceptable excipients to form pharmaceutical compositions.

Thus, POI variants identified as “hits” (e.g., POI variant that show favorable properties) in a first screen can be selected and systematically altered, e.g., using rational design, to optimize binding affinity, avidity, specificity, or other parameter. Such optimization can also be screened for using the methods described herein. Thus, in one embodiment, the disclosure includes screening a first library of POI variants using a method known in the art and/or described herein, identifying one or more hits in that library, subjecting those hits to systematic structural alteration to create a second library of POI variants structurally related to the hit, and screening the second library using the methods described herein.

POI variants identified as hits can be considered candidate therapeutic compounds (e.g., as a bio-therapeutic or as a delivery vehicle for a bio-therapeutic), useful in treating various disease or disorders (e.g., Type 1 and Type 2 diabetes mellitus, cancer, etc.). A variety of techniques useful for determining the structures of “hits” can be used in the methods described herein, e.g., NMR, mass spectrometry, gas chromatography equipped with electron capture detectors, fluorescence and absorption spectroscopy. Thus, the disclosure also includes POI variants identified as “hits” by the methods described herein, and methods for their administration and use (e.g., as a bio-therapeutic or as a delivery vehicle for a bio-therapeutic) in the treatment, prevention, management, or delay of development or progression of a disease or disorder.

POI variants identified as candidate therapeutic compounds (e.g., as a bio-therapeutic or as a delivery vehicle for a bio-therapeutic) can be further screened by administration to an animal model of a disease or disorder that can be treated and/or managed by the therapeutic compound. The animal can be monitored for a change in the disease or disorder, e.g., for an improvement in a parameter of the disease or disorder, e.g., a parameter related to clinical outcome. In some embodiments, the parameter is blood glucose or A1C level, and an improvement would be lower blood glucose or A1C level. In some embodiments, the subject is a human, e.g., a human with diabetes, and the parameter is blood glucose or A1C level.

### Screening of Delivery Vehicles

Polypeptide variants (e.g., POI variants) can be screened by the present methods to select variants as candidate delivery vehicles (e.g., variants that would be useful as delivery vehicles), such as delivery vehicles for bio-therapeutics, including, but not limited to, antisense oligonucleotides (ASO), small interfering RNAs (siRNAs), biologics (e.g., hormones, blood products, cytokines, growth factors, vaccines, gene and cellular therapies, fusion proteins, insulin, interferon, therapeutic antibodies or fragments thereof), etc. For example, variants of a known binder to a specific receptor of interest, including, but not limited to, GLP1R (glucagon like peptide 1 receptor), DPP6 (dipeptidyl peptidase like 6), and/or CCK-2 (cholecystokinin-2 receptor) can be screened by the present methods so as to identify variants of GLP1R binder, DPP6 binder, and/or CCK-2 binder as candidate delivery vehicles (e.g., variants that would be useful as delivery vehicles) for delivery of bio-therapeutics (e.g., ASO, siRNA, biologics, etc.) to pancreatic cells (e.g., pancreatic beta cells and/or cancerous pancreatic cells).

Utilizing the methods described herein, a pool of about 10² to about 10¹⁴ (e.g., about 10³ to about 10¹⁴, about 10⁴ to about 10¹⁴, about 10⁵ to about 10¹⁴, about 10⁶ to about 10¹⁴, about 10⁷ to about 10¹⁴, about 10⁸ to about 10¹⁴, about 10⁹ to about 10¹⁴, about 10¹⁰ to about 10¹⁴, about 10¹¹ to about 10¹⁴, about 10¹² to about 10¹⁴, about 10¹³ to about 10¹⁴, about 10² to about 10¹³, about 10³ to about 10¹³, about 10⁴ to about 10¹³, about 10⁵ to about 10¹³, about 10⁶ to about 10¹³, about 10⁷ to about 10¹³, about 10⁸ to about 10¹³, about 10⁹ to about 10¹³, about 10¹⁰ to about 10¹³, about 10¹¹ to about 10¹³, about 10¹² to about 10¹³, about 10² to about 10¹², about 10³ to about 10¹², about 10⁴ to about 10¹², about 10⁵ to about 10¹², about 10⁶ to about 10¹², about 10⁷ to about 10¹², about 10⁸ to about 10¹², about 10⁹ to about 10¹², about 10¹⁰ to about 10¹², about 10¹¹ to about 10¹², about 10² to about 10¹¹, about 10³ to about 10¹¹, about 10⁴ to about 10¹¹ about 10⁵ to about 10¹¹, about 10⁶ to about 10¹¹, about 10⁷ to about 10¹¹, about 10⁸ to about 10¹¹, about 10⁹ to about 10¹¹, about 10¹⁰ to about 10¹¹, about 10² to about 10¹⁰, about 10³ to about 10¹⁰, about 10⁴ to about 10¹⁰, about 10⁵ to about 10¹⁰, about 10⁶ to about 10¹⁰, about 10⁷ to about 10¹⁰, about 10⁸ to about 10¹⁰, about 10⁹ to about 10¹⁰, about 10² to about 10⁹, about 10³ to about 10⁹, about 10⁴ to about 10⁹, about 10⁵ to about 10⁹, about 10⁶ to about 10⁹, about 10⁷ to about 10⁹, or about 10⁸ to about 10⁹) POI variants can be made, each with a unique ssDNA barcode that is covalently attached, enabling simultaneous screening and selection of POI variants. The POI variants can be screened for efficient intracellular uptake and/or endosomal escape and/or traverse into the subcellular compartments of interest. The screening can be done in vivo (e.g., in an animal) and/or in vitro (e.g., in one cell/tissue culture well), followed by identification of the POI variant using its unique ssDNA barcode. The polypeptide library can be completely naïve. Alternatively, the polypeptide library can comprise variants of a known binder to a specific receptor of interest, including, but not limited to, transferrin receptor 1 (TfR1), glucagon like peptide receptor 1 (GLP1R), dipeptidyl peptidase like 6 (DPP6), low-density lipoprotein receptor (LDL-R), FXYD2, cholecystokinin-2 receptor (CCK-2), insulin receptor (IR), TMEM30A, angiotensin II type 1 receptor, ferroportin, neonatal Fc receptor (FcRn), megalin, cubilin, cd30, nectin-4, tissue factor, and LIV-1. By comparing the abundance of the unique ssDNA barcodes across different tissues, cell types, and/or subcellular compartments, the POI variants with the desired properties can be selected/identified as candidate delivery vehicles. A unique ssDNA barcode may be considered as abundant in one or more tissues, cell types, and/or subcellular compartments if that barcode is most abundantly (e.g., most abundant or frequent amongst all barcodes screened) found/recovered from those tissues, cell types, and/or subcellular compartments following a screening. Additionally, or in the alternative, a unique ssDNA barcode may be considered as abundant in one or more tissues, cell types, and/or subcellular compartments if that barcode comprises about 50% or more (e.g., about 50-60%, 60-70%, 70-80%, 80-90%, or 90-100% (e.g., about 50%, 55%, 60%, 65%, 70%, 75%, 80%, 85%, 90%, 95%, 96%, 97%, 98%, 99%, or 100%)) of all barcodes found/recovered from those tissues, cell types, and/or subcellular compartments following a screening. Additionally, or in the alternative, a unique ssDNA barcode may be considered as abundant in one or more tissues, cell types, and/or subcellular compartments if, following a screening, that barcode is found/recovered from those tissues, cell types, and/or subcellular compartments at a level that is higher than a threshold level, such as higher by about 5% or more (e.g., by about 5-10%, 10-20%, 20-30%, 30-40%, 40-50%, 50-60%, 60-70%, 70-80%, 80-90%, 90-100%, 100-200%, 200-300%, 300-400%, 400-500%, or more (e.g., by about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more)) over a threshold level.

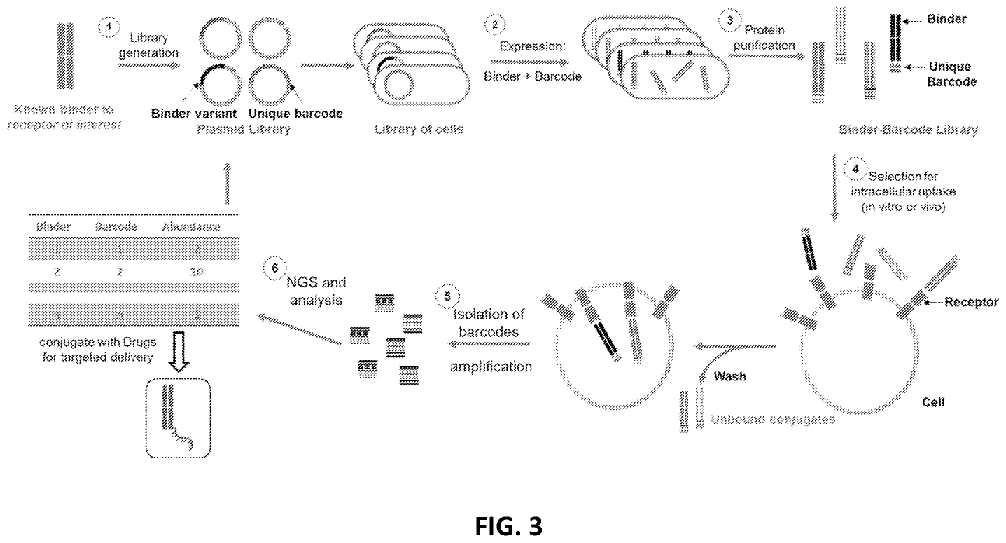

Data obtained from these multiplex experiments can be used to train machine learning models that can be applied to predict the function of new sequences and design new POI variants to be tested in subsequent experiments. Once the desired POI variants, i.e., the candidate delivery vehicles are identified, they can be coupled to any bio-therapeutic cargo, including oligonucleotides and proteins (e.g., ASOs, siRNAs, biologics, etc.) by appropriate linkers to generate targeted bio-therapeutics. Such targeted bio-therapeutics can then be used for treatment and/or management of diseases. A schematic representation of application of DEP library for screening of cell/tissue targeted delivery vehicles for bio-therapeutics is provided in FIG. 3.

### In Vivo Screening

For in vivo screening of POI variants by the methods of the present disclosure, a pool of POI-ssDNA conjugates (e.g., a pool of DEP) in a physiologically relevant vehicle can be administered into an animal. Examples of routes of administration include, without limitation, enteral administration (e.g., oral administration, sublingual or buccal administration, rectal administration, etc.), parenteral administration (e.g., by intravenous injection, intramuscular injection, subcutaneous injection, topical administration, transdermal administration, etc.), intranasal administration, administration by inhalation, and vaginal administration. For example, a pool of DEP (e.g., a DEP library) can be injected into an animal. The animal can be a mouse, rat, Guinea pig, or other rodent. The animal can also be from a higher non-human species, including, but not limited to a non-human primate, mini-pig, sheep, dog, etc. The ability to screen directly in a non-rodent species confers the present disclosure an advantage over currently available methods. For example, the higher species (e.g., non-rodent species) may better represent the human system, specifically, when delivery into the brain tissue is considered. One or more biological samples (e.g., tissues, blood and/or plasma) can be collected from the animal at pre-determined time points after administration of the DEP pool. The sample can be processed (e.g., tissues can be processed by homogenization or subcellular fractionation) to extract the DEP and associated barcodes. Barcodes in the tissue and/or cellular and/or subcellular fraction of interest can be identified by next generation sequencing. In some instances, a POI variant can be selected as a candidate delivery vehicle based on abundance of ssDNA barcode specific for the POI in the tissue and/or cellular and/or subcellular fraction of interest. For example, a POI variant whose unique barcode is most abundantly found/recovered from a particular tissue and/or cellular and/or subcellular fraction of interest, can be selected as a candidate delivery vehicle, i.e., as a POI variant that is most suitable (among the pool of POI variants screened) for use as a delivery vehicle.

### In Vitro Screening

For in vitro screening of POI variants by the methods of the present disclosure, a pool of POI-ssDNA conjugates (e.g., a pool of DEP) can be dosed to a cell culture. The pool of DEP can be dosed either in a solution or a suspension of a physiologically relevant vehicle. Samples can be obtained from the cell culture at several time points. The cells can then be processed, which includes either homogenization or subcellular fractionation to extract the DEP variants. Barcodes that remain in the cell and/or subcellular fraction of interest can then be identified by next generation sequencing. In some instances, a POI variant can be selected as a candidate delivery vehicle based on abundance of ssDNA barcode specific for the POI variant in the cell and/or subcellular fraction of interest. For example, a POI variant whose unique barcode is most abundantly found/recovered from a particular cell and/or subcellular fraction of interest, can be selected as a candidate delivery vehicle, i.e., as a POI variant that is most suitable (among the pool of POI variants screened) for use as a delivery vehicle.

### Screening of Protein Therapeutics

In certain instances, polypeptide variants (e.g., POI variants) can be screened by the present methods for potential use as bio-therapeutics, e.g., protein therapeutics for treatment and/or management of diseases. In certain instances when the polypeptide (e.g., POI) is a bio-therapeutic (e.g., a protein therapeutic, such as, a biologic), the desired POI variant identified from the screen can be expressed, purified, and/or used for treatment and/or management of diseases. POI variants that can be used as bio-therapeutics include, without limitation, factor VII, factor VIII, factor IX, factor X, GLP1R agonists, Iduronidase, Imiglucerase, Agalsidase alpha, Agalsidase beta, Alglucosidase alfa, Thymidine phosphorylase, Arginase-1, etc. In vivo and in vitro screening methods described in the foregoing can be employed for screening POI variants for potential use as bio-therapeutics.

In some instances, a POI variant can be identified as a suitable or effective bio-therapeutic, based on abundance of ssDNA barcode specific for the POI in a tissue and/or cell and/or cellular fraction and/or subcellular fraction of interest. For example, a POI variant whose unique barcode is most abundantly found/recovered from blood and/or plasma and/or a particular tissue and/or cell and/or cellular fraction and/or subcellular fraction of interest, can be identified as a POI that is most suitable (among the pool of POI variants screened) for use as a bio-therapeutic. A unique ssDNA barcode may be considered as abundant in blood and/or plasma and/or a particular tissue and/or a cell and/or a cellular fraction and/or a subcellular fraction if that barcode is most abundantly (e.g., most abundant or frequent amongst all barcodes screened) found/recovered from those blood and/or plasma and/or particular tissue and/or cell and/or cellular fraction and/or subcellular fraction, following a screening. Additionally, or in the alternative, a unique ssDNA barcode may be considered as abundant in blood and/or plasma and/or a particular tissue and/or a cell and/or a cellular fraction and/or a subcellular fraction if that barcode comprises about 50% or more (e.g., about 50-60%, 60-70%, 70-80%, 80-90%, or 90-100% (e.g., about 50%, 55%, 60%, 65%, 70%, 75%, 80%, 85%, 90%, 95%, 96%, 97%, 98%, 99%, or 100%)) of all barcodes found/recovered from blood and/or plasma and/or tissue and/or cell and/or cellular fraction and/or subcellular fraction, following a screening. Additionally, or in the alternative, a unique ssDNA barcode may be considered as abundant in blood and/or plasma and/or a particular tissue and/or a cell and/or a cellular fraction and/or a subcellular fraction, if, following a screening, that barcode is found/recovered from those blood and/or plasma and/or tissue and/or cell and/or cellular fraction and/or subcellular fraction, at a level that is higher than a threshold level, such as higher by about 5% or more (e.g., by about 5-10%, 10-20%, 20-30%, 30-40%, 40-50%, 50-60%, 60-70%, 70-80%, 80-90%, 90-100%, 100-200%, 200-300%, 300-400%, 400-500%, or more (e.g., by about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more)) over a threshold level.

In other instances, a POI variant can be identified as a suitable or effective bio-therapeutic, based on abundance of ssDNA barcode specific for the POI in blood and/or plasma and/or a tissue and/or cell and/or cellular fraction and/or subcellular fraction, at a specific time-point (e.g., about 15-30 minutes, 30-45 minutes, 45-60 minutes, 1-2 hours, 2-4 hours, 4-6 hours, 6-8 hours, 8-10 hours, 10-12 hours, 12-18 hours, 18-24 hours, 24-36 hours, 36-48 hours, 48-72 hours, 1-3 days, 3-6 days, 5-7 days, 1-2 weeks, 2-4 weeks, 1-2 months, 2-4 months, 4-6 months, 6-12 months, or more) after the pool of DEPs is administered to an animal and/or dosed to a cell culture. For example, a POI variant whose unique barcode is most abundantly found/recovered from blood and/or plasma and/or a particular tissue and/or cell and/or cellular fraction and/or subcellular fraction of interest at a specific time-point after a pool of DEPs is administered to an animal and/or dosed to a cell culture, can be identified as a POI variant with the longest half-life and can be considered as the most suitable POI variant (among the pool of POI variants screened) for use as a bio-therapeutic. In particular instances, a POI variant whose unique barcode is most abundantly found/recovered from plasma at a specific time-point after a pool of DEPs is administered to an animal, can be identified as a POI variant with the longest half-life in plasma and can be considered as the most suitable POI variant (among the pool of POI variants screened) for use as a bio-therapeutic.

### Screening of Biomarkers

In certain instances, the present methods can be employed to identify polypeptide variants (e.g., POI variants) that can be used to screen potential biomarkers. Such biomarkers can be used e.g., to evaluate disease state, to evaluate response to treatment, to predict response to treatment, or combinations thereof, wherein the disease state and/or response to treatment is associated with aberrant expression levels (e.g., high expression or low expression compared to control) of known protein biomarkers. In some instances, protein samples from biological samples of interest and control or comparison biological samples can be screened by the methods described hereinabove. The concept is based on immobilization of serum proteins (e.g., from biological samples of interest and control or comparison biological samples) onto magnetic beads, followed by target binding of ssDNA barcoded POI variants and a subsequent PCR step prior to detection by next generation sequencing (NGS), as described, for example, by Brofelth et al. (Commun Biol 3, 339 (2020)).

Briefly, biotinylated serum proteins (e.g., from biological samples of interest and control or comparison biological samples) can be captured and displayed on streptavidin-coated magnetic beads and a DEP library (e.g., a pool of DEPs) can be mixed with the beads. After washing the beads, adapter PCR can be performed to equip the DEPs with a sample-specific DNA tag (e.g., DNA tag specific for healthy sample(s) or DNA tag specific for disease sample(s)). PCR products obtained from the combined DEP and sample tags can then be analyzed by NGS. Once the barcodes enriched in the disease sample are identified by NGS, the associated POI variants can be expressed to capture and identify biomarkers for the disease.

### EXAMPLES

The invention is further described in the following examples, which do not limit the scope of the invention described in the claims.

### Nomenclature Used in the Following Examples

A: antibody, protein sequences
B: barcode
C: conjugate
D: single stranded DNA produced intracellularly
O: synthesized single stranded oligonucleotides
R: retron sequences

### Example 1. Intracellular Production of SUMO-PCV-ssDNA Conjugate with TRV-R-001

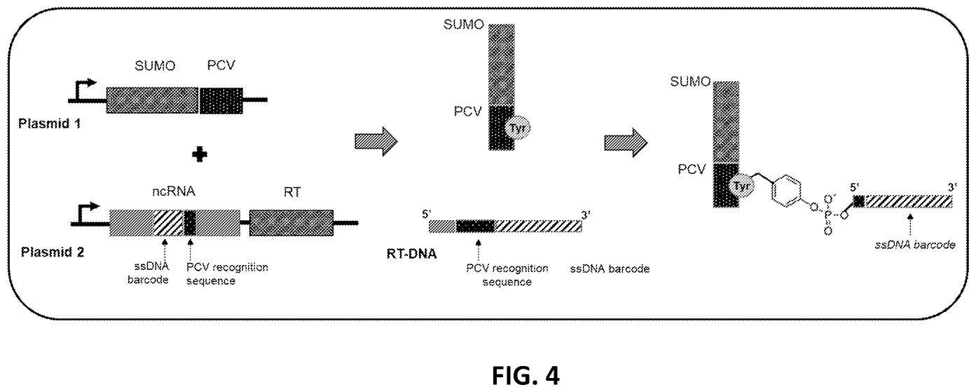

TRV-R-001 is derived from the retron EC86 with its native arrangements and consists of a reverse transcriptase (RT) and a downstream non-coding RNA (ncRNA) at 3′ end of the RT. The ncRNA contains an insertion in the stem loop that is transcribed by the RT into a single stranded RT-DNA, TRV-D-001. TRV-D-001 contains a DNA sequence recognized by the protein PCV and an ssDNA barcode, TRV-B-001. TRV-R-001 is cloned behind a T7/lac promoter in a vector based on pET28a (Kanamycin) with 5′ (NcoI) and 3′ (NotI). SUMO-PCV fusion protein sequence is cloned into pET21b (Ampicillin) with 5′ (NdeI) and 3′ (XhoI). Both plasmids are transformed into BL21-AI cells or BL21-AI strain without recJ and sbcB exonucleases. Cells are grown overnight and then diluted. RT-DNA and SUMO-PCV protein expression are induced during growth for 5-16 hours by addition of arabinose and IPTG. The abundance of ssDNA is quantified by relative qPCR, comparing amplification by primers that can amplify both the RT-DNA and plasmid, to amplification by primers that only amplify the plasmid. Ni-NTA beads are used to pull down his-tagged SUMO-PCV protein and the conjugation between SUMO-PCV and ssDNA are identified by a shift in molecular weight of SUMO-PCV and confirmed by analytical SEC. A schematic representation of intracellular production of SUMO-PCV-ssDNA conjugate with TRV-R-001 is provided in FIG. 4. The nucleic acid sequence of TRV-B-001 is provided below:

TRV-B-001:
(SEQ ID NO: 15)
5′-AAGTATTACCAGACGTGTGCTCTTCCGATCTGAGGGTACTTAGAT
CGGAAGAGCGTCGTGT-3′

### Example 2. Intracellular Production of SUMO-PCV-ssDNA Conjugate with TRV-R-002

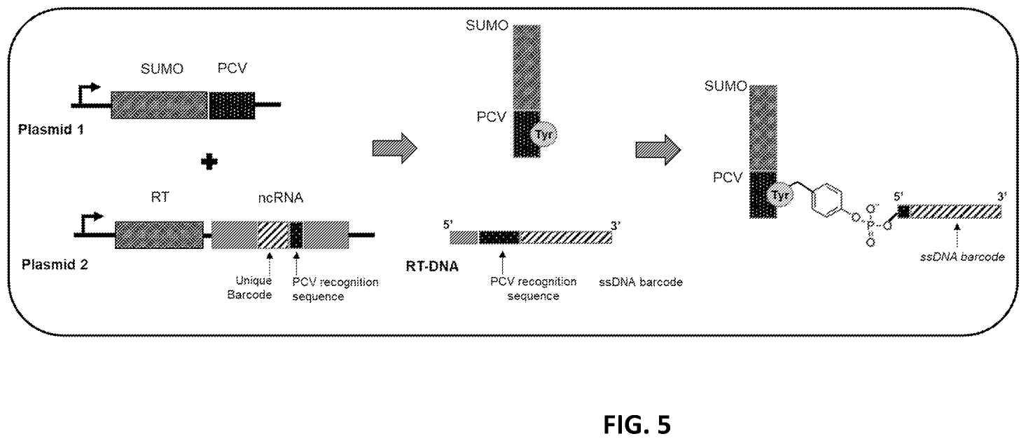

TRV-R-002 is derived from the retron EC86 with the operon inverted from its native arrangements and consists of a RT and an upstream ncRNA at 5′ end of the RT. The ncRNA contains an insertion in the stem loop that is transcribed by the RT into a single stranded RT-DNA, TRV-D-002. TRV-D-002 contains a DNA sequence recognized by the protein PCV and an ssDNA barcode, TRV-B-001. TRV-R-002 is cloned behind a T7/lac promoter in a vector based on pET28a (Kanamycin) with 5′ (NcoI) and 3′ (NotI). SUMO-PCV fusion protein sequence is cloned into pET21b (Ampicillin) with 5′ (NdeI) and 3′ (XhoI). Both plasmids are transformed into BL21-AI cells or BL21-AI strain without recJ and sbcB exonucleases. Cells are grown overnight and then diluted. RT-DNA and SUMO-PCV protein expression are induced during growth for 5-16 hours by addition of arabinose and IPTG. The abundance of ssDNA is quantified by relative qPCR, comparing amplification by primers that can amplify both the RT-DNA and plasmid, to amplification by primers that only amplify the plasmid. Ni-NTA beads are used to pull down his-tagged SUMO-PCV protein and the conjugation between SUMO-PCV and ssDNA are identified by a shift in molecular weight of SUMO-PCV and confirmed by analytical SEC. A schematic representation of intracellular production of SUMO-PCV-ssDNA conjugate with TRV-R-002 is provided in FIG. 5.

### Example 3. Generation of BL21-AI E. coli Strain ΔrecJ and ΔsbcB

BL21-AI E. coli ΔrecJ and ΔsbeB double knockout strain is constructed from the commercial BL21-AI E. coli strain using a two-plasmid CRISPR-Cas9 system. pCas in the two-plasmid system contains the cas9 gene with a native promoter, the A-Red recombination system to improve the editing efficiency, and the temperature-sensitive replication repA101 (Ts) for self-curing. pTargetF in the two-plasmid system consists of the sgRNA sequence, the N20 sequence, and the multiple restriction sites, and DNA sequence for homologous recombination. pCas plasmid is transformed into BL21-AI competent cells by electroporation. The successful transformants harboring pCas Plasmid are selected by kanamycin resistant. The synthesized sgRNA fragments are cloned into pTargetF plasmid by ligating into the A and B sites. The arms at the 5′ ends homologous to upstream and downstream chromosomal insert sites are cloned in to the sgRNA containing-pTarget plasmids. The sgRNA and homologous DNA containing pTarget plasmids are transformed into pCas harboring BL21-AI competent cells. Cells are recovered at 30° C. for 1 h before being spread onto LB agar containing kanamycin (50 mg/liter) and spectinomycin (50 mg/liter) and incubated overnight at 30° C. Transformants are characterized by colony PCR and Sanger DNA sequencing. The positive colony with ΔrecJ and ΔsbeB double knockout is cultured in presence of IPTG to cure the pTargetF plasmid, and the colonies cured of pTargetF are grown at 37° C. overnight to cure pCas plasmid. The strain is stored in glycerol at −80° C. for future application.

### Example 4. Generation of Library of GLP1 Peptides and Barcodes

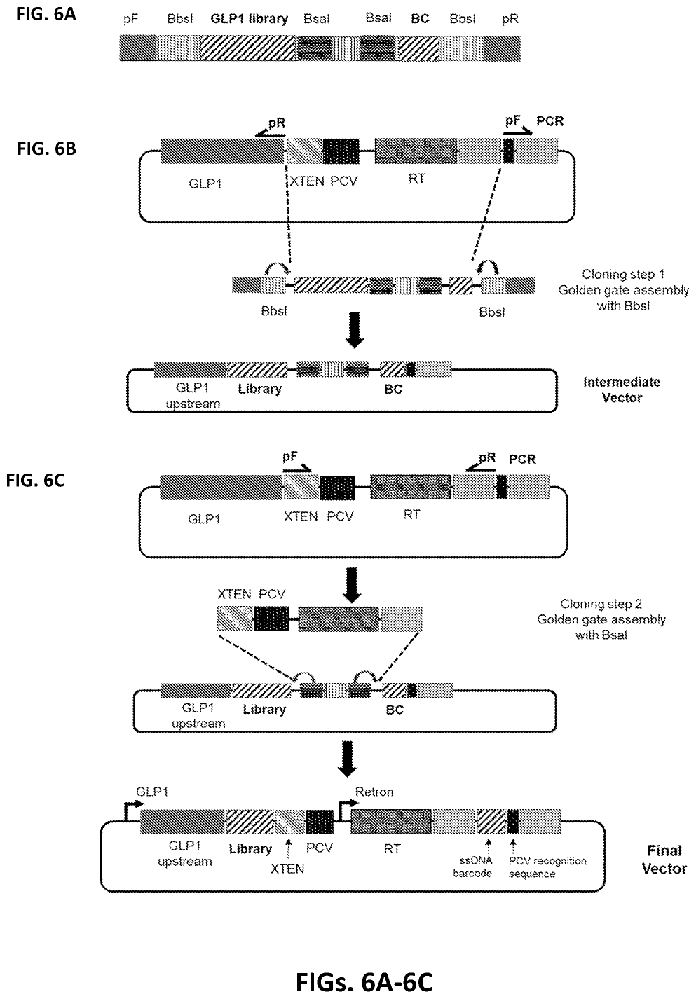

Oligos are synthesized on an Agilent oligo array (FIG. 6A), comprised of:
a. primers at both ends for amplification oligo pool
b. BbsI on flanking sites for golden gate cloning in step 1
c. GLP1 variant library
d. BsaI sites for golden gate cloning in step 2
e. EcoRV site between the two BsaI sites
f. Unique barcode associated with each GLP1 variant

Library cloning is performed in the following steps. First, the oligo is cloned into a linearized backbone containing the upstream GLP1 constant region and downstream HUH recognition site and 3′ region of the ncRNA of the retron system using Golden Gate Assembly (GGA), using BbsI-HF and T4 DNA ligase (FIG. 6B). The reaction is cycled 50-100× and then heat inactivated. This material is precipitated with ethanol, dissolved in TE buffer, combined with competent cells and electroporated. The cells are recovered in recovery media for 1 hour, grown overnight with media containing selective antibiotic, and then mini-prepped to obtain the intermediate vector. Next, the intermediate vector is linearized using two BsaI sites in opposing orientation. The gene region comprising of XTEN (to extend half-life of GLP1 agonist), PCV, and the retron system containing the RT (reverse transcriptase) and the 5′ region of the ncRNA is amplified and digested with BsaI. The two products are purified, mixed at 1:1 molar ratio, and ligated utilizing T4 ligase. The mixture is then digested with EcoRV which would cleave any undigested intermediate vector, precipitated with ethanol, dissolved in TE buffer, and electroporated into competent cells. The cells are recovered in recovery media for 1 hour, grown overnight with media containing selective antibiotic, and then mini-prepped to obtain the final vector (FIG. 6C).

### Example 5. Expression of DEP Library Containing GLP1 Peptides and Barcodes

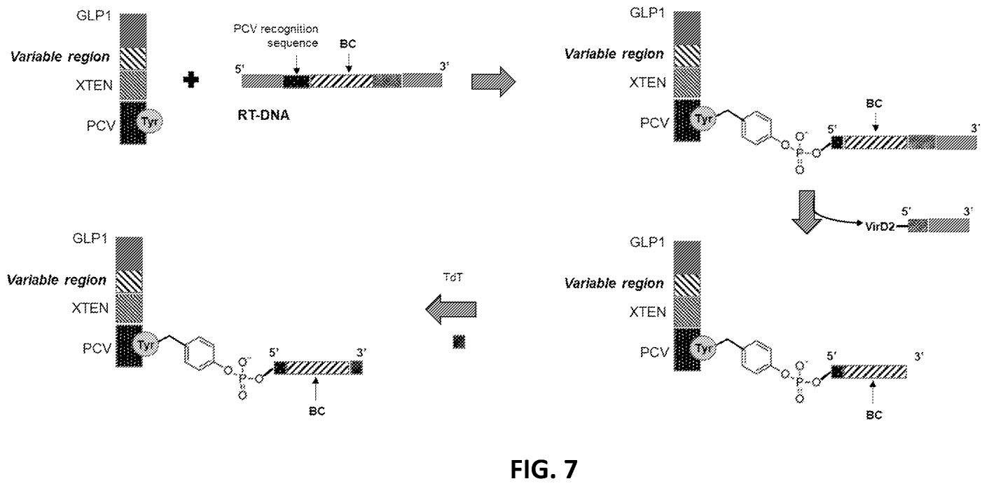

Library plasmids are transformed into BL21-AI cells or BL21-AI strain without recJ and sbcB exonucleases. Cells are grown overnight and then diluted. RT-DNA and GLP1-XTEN variant expression are induced during growth for 5-16 hours by adding inducers. Cells are pelleted and lysed in 50 mM Tris, 200 mM NaCl, 20% sucrose, pH 7.4. The clarified supernatant is first passed over Ni-NTA resin, washed with 50 mM Tris, 200 mM NaCl, 15 mM imidazole, pH 7.4 and eluted with 250-500 mM imidazole. Combined fractions are dialyzed and concentrated into 50 mM HEPES, 150 mM NaCl, 10% glycerol, 1 mM DTT, pH 7.4. VirD2 is added to remove the non-functional ssDNA region. TdT (Terminal Deoxynucleotidyl Transferase) and Biotin-11-ddUTP are added to cap and protect the barcode from exonucleases in vitro or in vivo. A schematic representation of expression of DEP library containing GLP1 peptides and barcodes is provided in FIG. 7.

### Example 6. Delivery of ssDNA Barcodes into K562 Cells with a TfR1 Targeting Complex

A TfR1 targeting complex is generated comprising the barcode mixture A (mixture of barcodes TRV-O-002, 003, 004, and 005 at ratios of 1000:100:10:1, respectively) covalently linked to TRV-P-001 (OKT9 Fab), an anti-transferrin receptor antibody, via PCV.

Equimolar amounts of TRV-P-001-PCV and the barcode mixture A are incubated at room temperature for 60 minutes in pH 7.4 buffer containing 1 mM MgCl2. Confirmation of the linkage is analyzed by SDS-PAGE. The product of the Fab coupling is then subjected to size-exclusion chromatography (SEC). Fractions containing the Fab-oligonucleotide complex (referred to as TRV-C-001) are combined and concentrated.

Using the same methods as described above, a control complex is generated comprising the barcode TRV-O-001 covalently linked via PCV to an IgG1 (Fab) antibody (TRV-C-002).

The purified TRV-C-001 is then tested for cellular internalization. K562 cells, which have relatively high expression levels of transferrin receptor, are incubated in the presence of vehicle control, TRV-C-001 (10-1000 nM), or TRV-C-002 (10-1000 nM) for 1-5 hours. After incubation, the cells are isolated, lysed, and the oligonucleotides are pulled down by streptavidin magnetic beads. Crude oligonucleotides are further purified and amplified by PCR containing oligonucleotide pool, dNTPs, Universal primer, Index primer, Index-base primer, Phusion enzyme (New England Biolabs), DMSO, HF Phusion buffer, and H20. PCR products are run by gel electrophoresis on 1.4% Tris-acetate-EDTA agarose, and bands are excised, pooled, and purified by Zymo Gel Extraction columns. Agarose bands containing PCR products were pooled only if the Index primers are distinct. The purified products are kept frozen until deep sequencing. Deep-sequencing runs are performed using multiplexed runs on Illumina Miseq machines.

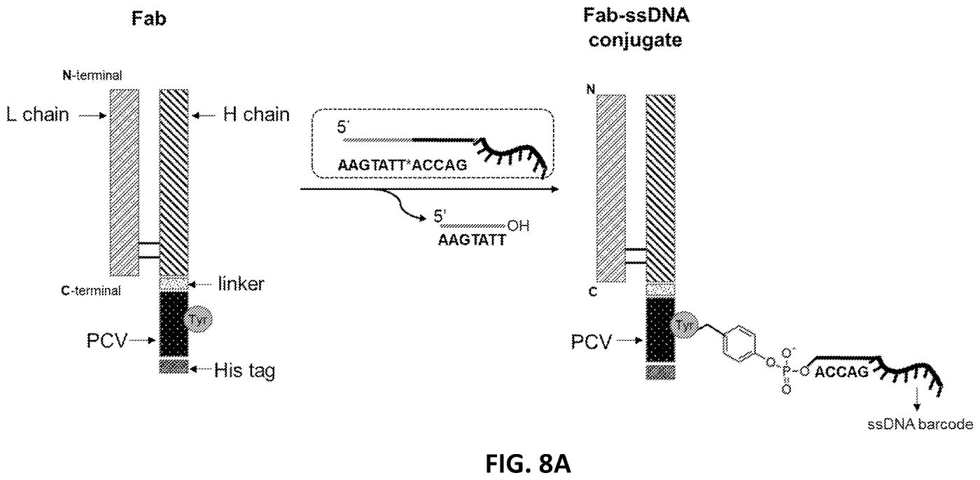

A schematic representation of an exemplary method of delivery of ssDNA barcodes into K562 cells with a TfR1 targeting complex is provided in FIGS. 8A-8B.

### Example 7. SUMO-PCV Fusion is Active when Expressed in E. coli

The activity of an exemplary SUMO-PCV fusion protein was tested in E. coli. Due to the ease of its expression, SUMO was selected as a protein representative of a typical protein to be displayed by this technology or as the protein of interest (POI) for the proof-of-concept study. The endonuclease domain from the Rep protein of porcine circovirus type 2 (PCV) was selected as the HUH-tag, because it is one of the smallest HUH-tags (13 kDa), is well characterized, and is representative of the potential HUH-tags that may be used to express POI with the methods described herein.

To confirm that the SUMO-PCV fusion protein is functional and can form covalent adducts with a desired synthetic ssDNA, SUMO-PCV protein with C-terminal His-tag was expressed in BL21-AI E. coli. Cell lysate containing SUMO-PCV were incubated with TRV-O-001, a single-stranded oligo bearing PCV's target sequence (AAGTATTACC; SEQ ID NO: 37) and a barcode. Briefly, linear coding DNA was inserted into vector pET21b at the 5′ NdeI and 3′ XhoI sites. The construct was transformed into E. coli BL21-AI cells and grown in TB supplemented with 100 μg/mL ampicillin. The cells were induced at OD600 of 0.6 with 0.5 mM isopropyl-D-1-thiogalactopyranoside (IPTG) and 0.2% L-arabinose overnight at 25° C. Cells were harvested and suspended in 5% POPCULTURE reagent in lysis buffer (20 mM Tris-HCl pH 8.0, 200 mM NaCl, 5% glycerine, 1 mM TCEP, 2 mM MgCl₂, Roche protease inhibitor tablet, 0.2 mM PMSF) for 30 minutes at 4° C. and centrifuged at 12000 rpm for 30 minutes at 4° C. Soluble fraction was collected and combined with TRV-O-001 (5 μM final concentration) and incubated for 1 hour at room temperature. The mixture was added to nickel-NTA agarose (Thermo Scientific), incubated for 1 hour at 4° C., and washed with wash buffer (20 mM Tris-HCl pH 8.0, 200 mM NaCl, 1 mM TCEP, 5% glycerine, 2 mM MgCl₂, 10 mM Imidazole). Proteins were eluted with wash buffer containing 300 mM imidazole and analyzed by SDS-PAGE and western blot (using anti-His antibody).

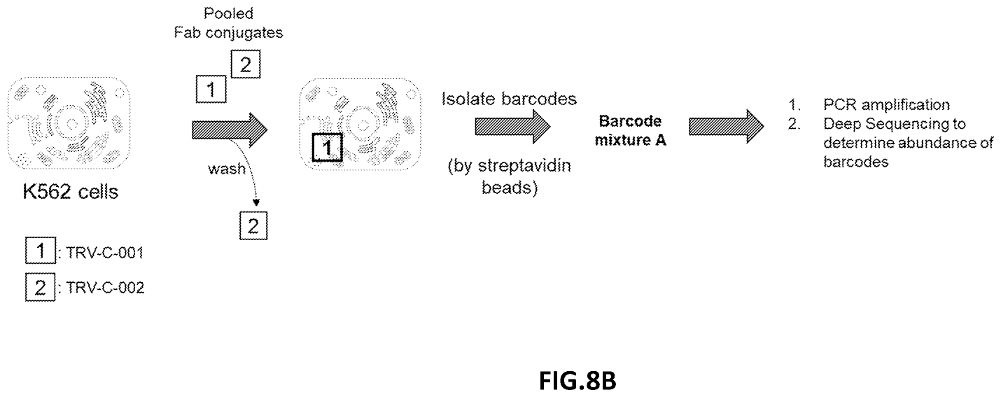

As shown in FIG. 9, there was complete disappearance of SUMO-PCV and formation of the desired covalent adduct ssDNA-SUMO-PCV, which ran higher on both the SDS-PAGE and the western blot. This demonstrated that the SUMO-PCV fusion protein is functional and can form covalent adducts with a desired synthetic SSDNA.

### Example 8. E. coli can Express TRV-D-001, an ssDNA Comprising a Barcode and the PCV Target Sequence

To test if the retron system can express TRV-D-001 (an ssDNA containing the PCV target sequence (AAGTATTACC; SEQ ID NO: 37) and an amplifiable barcode), the wild type Eco1 retron (also known as ec86) was engineered with the reverse complement of TRV-D-001 sequence inserted in the loop of the msd and cloned into pET28a vector for expression in BL21-AI E. coli.

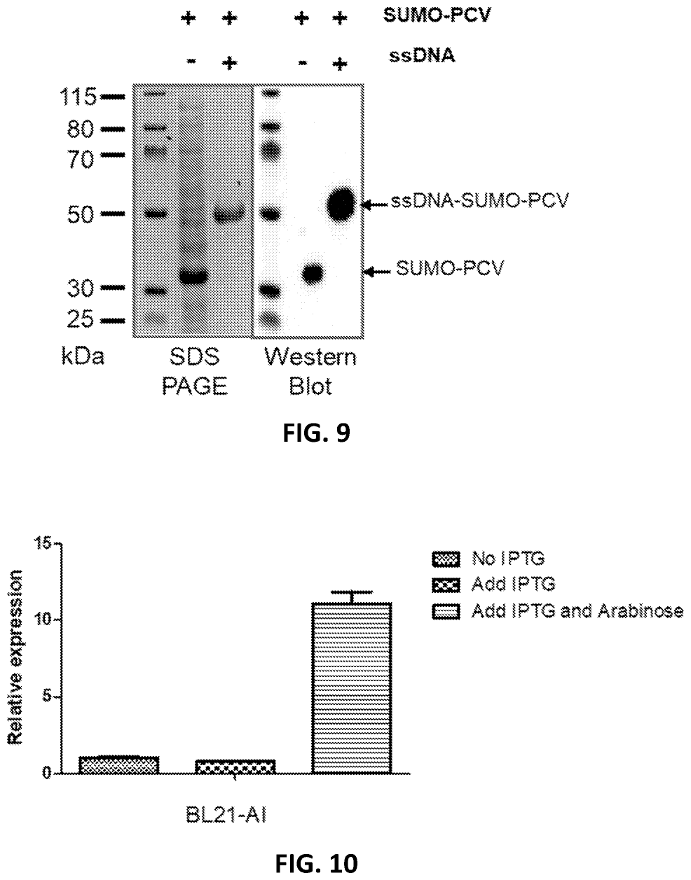

Briefly, TRV-R-003 was derived from the retron EC86 with its native arrangements and contains a non-coding RNA (ncRNA) and a downstream reverse transcriptase (RT). The ncRNA contains an insertion in the stem loop in the msd that can be transcribed by the RT into a single stranded RT-DNA containing the desired SSDNA TRV-D-001. TRV-R-003 was cloned behind a T7/lac promoter into pET28a (Kanamycin) at the 5′ NcoI and 3′ NotI sites. The construct was transformed into E. coli BL21-AI cells and grown in LB media. The cells were induced at OD600 of 0.6 with 0.5 mM isopropyl-D-1-thiogalactopyranoside (IPTG) and 0.2% L-arabinose overnight at 18° C. The ssDNA production was evaluated by qPCR comparing the relative amplification from samples using two sets of primers. One set bound inside the msd can amplify both RT-DNA and plasmid as the template. The other set bound outside the msd in the RT can only amplify the plasmid. The ssDNA production was evaluated by qPCR comparing the relative amplification from samples using the two sets of primers. Results were analyzed by first taking the difference in cycle threshold (CT) between the inside and outside primer sets for each replicate. Each replicate ΔCT was subtracted from the average ΔCT of the control condition (e.g., uninduced). Fold change was calculated as 2^{-ΔΔCT}. The results are described in FIG. 10. Sequence of the Retron and the primer sets are provided below.

As shown in FIG. 10, overexpression of the ncRNA and RT from the plasmid with both inducers (IPTG and Arabinose) yielded about ˜10-fold enrichment of the RT-DNA/plasmid region over the plasmid alone.

Retron TRV-R-003 construct:
(SEQ ID NO: 38)
ATGCGCACCCTTAGCGAGAGGTTTATCATTAAGGTCAACCTCTGGATGT
TGTTTCGGCATCCTGCATTGAATCTGAGTTACTGTCTGTTTTCCTTGTT
GGAACGGAGAGCATCacacgacgctcttccgatctaagtaccctcagat
cggaagagcacacgtctggtaatacttGATGCTCTCCGAGCCAACCAGG
AAACCCGTTTTTTCTGACGTAAGGGTGCGCAACTTTCATGAAATCCGCT
GAATATTTGAACACTTTTAGATTGAGAAATCTCGGCCTACCTGTCATGA
ACAATTTGCATGACATGTCTAAGGCGACTCGCATATCTGTTGAAACACT
TCGGTTGTTAATCTATACAGCTGATTTTCGCTATAGGATCTACACTGTA
GAAAAGAAAGGCCCAGAGAAGAGAATGAGAACCATTTACCAACCTTCTC
GAGAACTTAAAGCCTTACAAGGATGGGTTCTACGTAACATTTTAGATAA
ACTGTCGTCATCTCCTTTTTCTATTGGATTTGAAAAGCACCAATCTATT
TTGAATAATGCTACCCCGCATATTGGGGCAAACTTTATACTGAATATTG
ATTTGGAGGATTTTTTCCCAAGTTTAACTGCTAACAAAGTTTTTGGAGT
GTTCCATTCTCTTGGTTATAATCGACTAATATCTTCAGTTTTGACAAAA
ATATGTTGTTATAAAAATCTGCTACCACAAGGTGCTCCATCATCACCTA
AATTAGCTAATCTAATATGTTCTAAACTTGATTATCGTATTCAGGGTTA
TGCAGGTAGTCGGGGCTTGATATATACGAGATATGCCGATGATCTCACC
TTATCTGCACAGTCTATGAAAAAGGTTGTTAAAGCACGTGATTTTTTAT
TTTCTATAATCCCAAGTGAAGGATTGGTTATTAACTCAAAAAAAACTTG
TATTAGTGGGCCTCGTAGTCAGAGGAAAGTTACAGGTTTAGTTATTTCA
CAAGAGAAAGTTGGGATAGGTAGAGAAAAATATAAAGAAATTAGAGCAA
AGATACATCATATATTTTGCGGTAAGTCTTCTGAGATAGAACACGTTAG
GGGATGGTTGTCATTTATTTTAAGTGTGGATTCAAAAAGCCATAGGAGA
TTAATAACTTATATTAGCAAATTAGAAAAAAAATATGGAAAGAACCCTT
TAAATAAAGCGAAGACCTAA

Italics: non-coding RNA
lowercase and italics: sequence encoding for TRV-D-001
Bold and italics: binding sites for primer pair to amplify both the RT-DNA and plasmid
Bold: binding sites for primer pair to amplify only the plasmid
Normal: reverse transcriptase
Primer pair to amplify the plasmid alone:

F Primer:
(SEQ ID NO: 20)
TACCACAAGGTGCTCCATCA
R Primer:
(SEQ ID NO: 21)
TATCAAGCCCCGACTACCTG

Primer pair to amplify both the RT-DNA and plasmid:

F Primer:
(SEQ ID NO: 22)
ACTGTCTGTTTTCCTTGTTGG
R Primer:
(SEQ ID NO: 23)
AACGGGTTTCCTGGTTGG

### Example 9. Formation of ssDNA-SUMO-PCV Conjugate Confirmed by qPCR

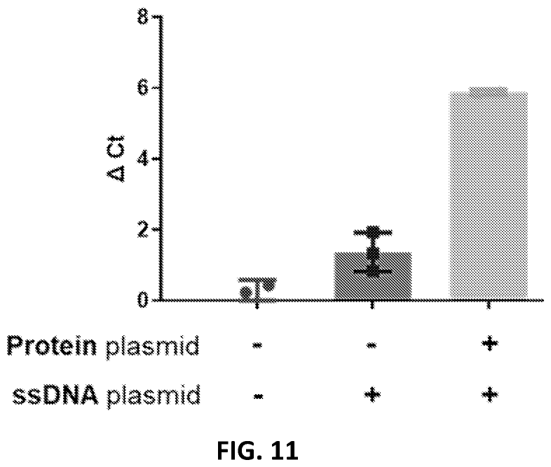

Having confirmed that the individual plasmids functioned as intended, both plasmids were then co-expressed to test if ssDNA-SUMO-PCV conjugate was formed in cells. Briefly, both plasmids encoding for ssDNA TRV-D-001 and SUMO-PCV fusion protein were transformed into E. coli BL21-AI cells and grown in TB media. The cells were induced at OD600 of 0.6 with 0.5 mM isopropyl-D-1-thiogalactopyranoside (IPTG) and 0.2% L-arabinose overnight at 25° C. Cells were harvested and suspended in 5% POPCULTURE reagent in lysis buffer (20 mM Tris-HCl pH 8.0, 200 mM NaCl, 5% glycerine, 1 mM TCEP, 2 mM MgCl₂, Roche protease inhibitor tablet, 0.2 mM PMSF) for 30 minutes at 4° C. and centrifuged at 12000 rpm for 30 minutes at 4° C. Soluble fraction was collected and added to nickel-NTA agarose (Thermo Scientific), incubated for 1 hour at 4° C., and washed with wash buffer (20 mM Tris-HCl pH 8.0, 200 mM NaCl, 1 mM TCEP, 5% glycerine, 2 mM MgCl₂, 10 mM Imidazole). Desired ssDNA-SUMO-PCV conjugate and SUMO-PCV protein were eluted with wash buffer containing 300 mM imidazole and analyzed by qPCR using primers for the presence of TRV-D-001. The results are described in FIG. 11. Sequence of the primers used for the qPCR analysis are provided below.

As described in FIG. 11, qPCR analysis of the eluted fractions showed an increased amplification only when both plasmids were present, indicating that the desired conjugate (ssDNA-SUMO-PCV) was formed.

F Primer:
(SEQ ID NO: 39)
AGACGTGTGCTCTTCCGATCT
R Primer:
(SEQ ID NO: 40)
ACACGACGCTCTTCCGATCT

### REFERENCES

1. Schubert et al., High-throughput functional variant screens via in vivo production of single-stranded DNA. Proc Natl Acad Sci 118 (18) e2018181118 (2021).
2. Brofelth et al., Multiplex profiling of serum proteins in solution using barcoded antibody fragments and next generation sequencing. Commun Biol 3, 339 (2020).
3. Sugo et al., Development of antibody-siRNA conjugate targeted to cardiac and skeletal muscles. Journal of Controlled Release 237:1-13 (2016).
4. Lovendahl et al., Sequence-directed covalent protein-DNA linkages in a single step using HUH-tags. J Am Chem Soc 139 (20): 7030-7035 (2017).
5. Hu et al., Click-type protein-DNA conjugation for Mn2+ imaging in living Cells. Anal Chem 91 (15): 10180-10187 (2019).
6. Roberts et al., Advances in oligonucleotide drug delivery. Nat Rev Drug Discov 19:673-694 (2020).
7. International Publication Number WO 2021/051011 A1.
8. United States Patent Number U.S. Pat. No. 10,717,773 B2.
9. United States Patent Application Publication Number US 2019/0275104 A1.
10. United States Patent Application Publication Number US 2019/0346456 A1.
11. United States Patent Application Publication Number US 2021/0156048 A1.

Representative nucleic acid and amino acid sequences that can be used in the compositions and methods of the present disclosure are provided in the Sequence Table below.

SEQUENCE TABLE

| | | Sequence |
|---|---|---|
| Description | Sequence | Identifier |
| PCV | MPSKKNGRSGPQPHKRWVFTLNNPSEDERKKIR | SEQ ID |
| | DLPISLFDYFIVGEEGNEEGRTPHLQGFANFVKK | NO: 1 |
| | QTFNKVKWYLGARCHIEKAKGTDQQNKEYCSK | |
| | EGNLLMECGAPRSQGQR | |
| DCV | MAKSGNYSYKRWVFTINNPTFEDYVHVLEFCTL | SEQ ID |
| | DNCKFAIVGEEKGANGTPHLQGFLNLRSNARAA | NO: 2 |
| | ALEESLGGRAWLSRARGSDEDNEEYCAKESTYL | |
| | RVGEPVSKGRSSDLAEATSAV | |
| VirD2 | MPDRAQVIIRIVPGGGTKTLQQIINQLEYLSRKGK | SEQ ID |
| | LELQRSARHLDIPVPPDQIRELAQSWVTEAGIYDE | NO: 3 |
| | SQSDDDRQQDLTTHIIVSFPAGTDQTAAYEASRE | |
| | WAAEMFGSGYGGGRYNYLTAYHVDRDHPHLH | |
| | VVVNRRELLGHGWLKISRRHPQLNYDGLRKKM | |
| | AEISLRHGIVLDATSRAERGIAERPITYAEHRRLE | |
| | RMQPW | |
| RepB | MAKEKARYFTFLLYPESIPSDWELKLETLGVPMA | SEQ ID |
| | ISPLHDKDKSSIKGQKYKKAHYHVLYIAKNPVTA | NO: 4 |
| | DSVRKKIKLLLGEKSLAMVQVVLNVENMYLYLT | |
| | HESKDAIAKKKHVYDKADIKLINNFDIDRY | |
| TraI | MMSIAQVRSAGSAGNYYTDKDNYYVLGSMGER | SEQ ID |
| | WAGRGAEQLGLQGSVDKDVFTRLLEGRLPDGA | NO: 5 |
| | DLSRMQDGSNRHRPGYDLTFSAPKSVSMMAML | |
| | GGDKRLIDAHNQAVDFAVRQVEALASTRVMTD | |
| | GQSETVLTGNLVMALFNHDTSRDQEPQLHTHAV | |
| | VANVTQHNGEWKTLSSDKVGKTGFIENVYANQI | |
| | AFGRLYREKLKEQVEALGYETEVVGKHGMWEM | |
| | PGVPVEAFSGRSQTIREAVGEDASLKSRDVAALD | |
| | TRKSKQHVDPEIKMAEWMQTLKETGFDIRAYRD | |
| | AADQRADLRTLTPGPASQDGPDVQQAVTQAIAG | |
| | LSER | |
| mMobA | MAIYHLTAKTGSRSGGQSARAKADYIQREGKYA | SEQ ID |
| | RDMDEVLHAESGHMPEFVERPADYWDAADLYE | NO: 6 |
| | RANGRLFKEVEFALPVELTLDQQKALASEFAQH | |
| | LTGAERLPYTLAIHAGGGENPHCHLMISERINDGI | |
| | ERPAAQWFKRYNGKTPEKGGAQKTEALKPKAW | |
| | LEQTREAWADHANRALERAGH | |
| NES | MAMYHFQNKFVSKANGQSATAKSAYNSASRIK | SEQ ID |
| | DFKENEFKDYSNKQCDYSEILLPNNADDKFKDR | NO: 7 |
| | EYLWNKVHDVENRKNSQVAREIIIGLPNEFDPNS | |
| | NIELAKEFAESLSNEGMIVDLNIHKINEENPHAHL | |
| | LCTLRGLDKNNEFEPKRKGNDYIRDWNTKEKHN | |
| | EWRKRWENVQNKHLEKNGFSVRVSADSYKNQN | |
| | IDLEPTKKEGWKARKFEDETG | |
| ssDNA recognition | aagtattaccagaaa | SEQ ID |
| sequence for PCV | | NO: 8 |
| ssDNA recognition | aagtattaccag | SEQ ID |
| sequence for DCV | | NO: 9 |
| ssDNA recognition | caacggtatatatcctgcca | SEQ ID |
| sequence for VirD2 | | NO: 10 |
| ssDNA recognition | tgcttccgtactacgacccccca | SEQ ID |
| sequence for RepB | | NO: 11 |
| ssDNA recognition | tttgcgtggggtgtggtgcttt | SEQ ID |
| sequence for Tral | | NO: 12 |
| ssDNA recognition | ccagtttctcgaagagaaaccggtaagtgcaccctccc | SEQ ID |
| sequence for mMobA | | NO: 13 |
| ssDNA recognition | acgcgaacggaacgttcgcataagtgcgcccttacgggatttaac | SEQ ID |
| sequence for NES | | NO: 14 |
| TRV-O-001 | AAGTATTACCAGACGTGTGCTCTTCCGATCTGAG | SEQ ID |
| | GGTACTTAGATCGGAAGAGCGTCG*T*G*T- | NO: 15 |
| | biotin | |
| TRV-O-002 | AAGTATTACCAGACGTGTGCTCTTCCGATCTGAC | SEQ ID |
| | AATTGCCAGATCGGAAGAGCGTCG*T*G*T- | NO: 16 |
| | biotin | |
| TRV-O-003 | AAGTATTACCAGACGTGTGCTCTTCCGATCTTAA | SEQ ID |
| | CGCACCTAGATCGGAAGAGCGTCG*T*G*T- | NO: 17 |
| | biotin | |
| TRV-O-004 | AAGTATTACCAGACGTGTGCTCTTCCGATCTATG | SEQ ID |
| | ATCGTCGAGATCGGAAGAGCGTCG*T*G*T- | NO: 18 |
| | biotin | |
| TRV-O-005 | AAGTATTACCAGACGTGTGCTCTTCCGATCTTGT | SEQ ID |
| | CTCCCATAGATCGGAAGAGCGTCG*T*G*T- | NO: 19 |
| | biotin | |
| F primer for qPCR | TACCACAAGGTGCTCCATCA | SEQ ID |
| quantification for | | NO: 20 |
| plasmid | | |
| R primer for qPCR | TATCAAGCCCCGACTACCTG | SEQ ID |
| quantification for | | NO: 21 |
| plasmid | | |
| F primer for qPCR | ACTGTCTGTTTTCCTTGTTGG | SEQ ID |
| quantification for | | NO: 22 |
| RT-DNA and | | |
| plasmid | | |
| R primer for qPCR | AACGGGTTTCCTGGTTGG | SEQ ID |
| quantification for | | NO: 23 |
| RT-DNA and | | |
| plasmid | | |
| Universal Reverse | AATGATACGGCGACCACCGAGATCTACACAGG | SEQ ID |
| Primer for deep | CTATAACACTCTTTCCCTACACGACGCTCTTCC | NO: 24 |
| sequencing | GATCT | |
| Index Base Forward | TGACTGGAGTTCAGACGTGTGCTCTTCCGATCT | SEQ ID |
| Primer for deep | | NO: 25 |
| sequencing | | |
| Index Final Forward | CAAGCAGAAGACGGCATACGAGATGGCAGATA | SEQ ID |
| Primer for deep | GTGACTGGAGTTCAGACGTGTG | NO: 26 |
| sequencing | | |
| VH of TRV-A-001 | EVQLQQSGPELVKPGTSMNISCKASGYTFTNYY | SEQ ID |
| (Fab anti-human | MHWVRQRPGQGLEWIGWIYPGDGNTHYNEKFK | NO: 27 |
| transferrin receptor | GKTTLTADTSSITAYMLLSSLTSEDSAVYFCARD | |
| 1-PCV (mouse IgG1, | NSGYVGFPYWGQGTTLTVSSAKTTPPSVYPLAPG | |
| kappa)) | SAAQTNSMVTLGCLVKGYFPEPVTVTWNSGSLS | |
| | SGVHTFPAVLQSDLYTLSSSVTVPSSPRPSETVTC | |
| | NVAHPASSTKVDKKIVPRDCGGGSGGGSGGGSP | |
| | SKKNGRSGPQPHKRWVFTLNNPSEDERKKIRDLP | |
| | ISLFDYFIVGEEGNEEGRTPHLQGFANFVKKQTF | |
| | NKVKWYLGARCHIEKAKGTDQQNKEYCSKEGN | |
| | LLMECGAPRSQGQRPKKKRKVHHHHHH | |
| VL of TRV-A-001 | DIKMTQSPSSLTVTAGERVTMSCKSSQSLFNSAN | SEQ ID |
| (Fab anti-human | QENYLTWYQQRPGQPPKLLIYWASTRDSGVPDR | NO: 28 |
| transferrin receptor | FTGSGSGTDFPLTISSVQAEDLAVYYCQNDYSYP | |
| 1-PCV (mouse IgG1, | LTFGAGTKLEIKADAAPTVSIFPPSSEQLTSGGAS | |
| kappa)) | VVCFLNNFYPKDINVKWKIDGSERQNGVLNSWT | |
| | DQDSKDSTYSMSSTLTLTKDEYERHNSYTCEAT | |
| | HKTSTSPIVKSFNRGEC | |
| VH of TRV-A-002 | EVQLVESGGGLVQPGNSLTLSCVASGFTFSNYG | SEQ ID |
| (Fab anti-mouse | MHWIRQAPKKGLEWIAMIYYDSSKMNYADTVK | NO: 29 |
| transferrin receptor | GRFTISRDNSKNTLYLEMNSLRSEDTAMYYCAV | |
| 1-PCV (mouse IgG1, | PTSHYVVDVWGQGVSVTVSSAKTTPPSVYPLAP | |
| kappa)) | GSAAQTNSMVTLGCLVKGYFPEPVTVTWNSGSL | |
| | SSGVHTFPAVLQSDLYTLSSSVTVPSSPRPSETVT | |
| | CNVAHPASSTKVDKKIVPRDCGGGSGGGSGGGS | |
| | PSKKNGRSGPQPHKRWVFTLNNPSEDERKKIRDL | |
| | PISLFDYFIVGEEGNEEGRTPHLQGFANFVKKQTF | |
| | NKVKWYLGARCHIEKAKGTDQQNKEYCSKEGN | |
| | LLMECGAPRSQGQRPKKKRKVHHHHHH | |
| VL of TRV-A-002 | DIQMTQSPASLSASLEEIVTITCQASQDIGNWLA | SEQ ID |
| (Fab anti-mouse | WYQQKPGKSPQLLIYGATSLADGVPSRFSGSRSG | NO: 30 |
| transferrin receptor | TQFSLKISRVQVEDIGIYYCLQAYNTPWTFGGGT | |
| 1-PCV (mouse IgG1, | KLELKRADAAPTVSIFPPSSEQLTSGGASVVCFLN | |
| kappa)) | NFYPKDINVKWKIDGSERQNGVLNSWTDQDSKD | |
| | STYSMSSTLTLTKDEYERHNSYTCEATHKTSTSPI | |
| | VKSFNRGEC | |
| SUMO-PCV protein | MASGSDSEVNQEAKPEVKPEVKPETHINLKVSD | SEQ ID |
| sequence | GSSEIFFKIKKTTPLRRLMEAFAKRQGKEMDSLR | NO: 31 |
| | FLYDGIRIQADQTPEDLDMEDNDIIEAHREQIGGS | |
| | PSKKNGRSGPQPHKRWVFTLNNPSEDERKKIRDL | |
| | PISLFDYFIVGEEGNEEGRTPHLQGFANFVKKQTF | |
| | NKVKWYLGARCHIEKAKGTDQQNKEYCSKEGN | |
| | LLMECGAPRSQGQRPKKKRKVHHHHHH | |
| SUMO-PCV gene | CATATGGCGAGCGGCAGCGATAGCGAAGTGAA | SEQ ID |
| sequence | CCAAGAAGCGAAACCGGAAGTGAAGCCGGAA | NO: 32 |
| | GTGAAGCCGGAAACCCATATTAACCTGAAAGT | |
| | GAGCGATGGCAGCAGCGAAATTTTTTTTAAAA | |
| | TTAAAAAAACCACCCCGCTGCGCCGCCTGATG | |
| | GAAGCGTTTGCGAAACGCCAAGGCAAAGAAAT | |
| | GGATAGCCTGCGCTTTCTGTATGATGGCATTCG | |
| | CATTCAAGCGGATCAGACCCCGGAAGATCTGG | |
| | ATATGGAAGATAACGATATTATTGAAGCGCAT | |
| | CGCGAACAGATTGGCGGCAGCCCGAGCAAAA | |
| | AAAACGGCCGCAGCGGCCCGCAGCCGCATAAA | |
| | CGCTGGGTGTTTACCCTGAACAACCCGAGCGA | |
| | AGATGAACGCAAGAAAATTCGCGATCTGCCGA | |
| | TTAGCCTGTTTGATTATTTTATTGTGGGCGAAG | |
| | AAGGCAACGAAGAAGGCCGCACCCCGCATCTG | |
| | CAAGGCTTTGCGAACTTTGTGAAAAAACAGAC | |
| | CTTTAACAAAGTGAAATGGTATCTGGGCGCGC | |
| | GCTGCCATATTGAAAAAGCGAAAGGCACCGAT | |
| | CAGCAGAACAAAGAATATTGCAGCAAAGAAG | |
| | GCAACCTGCTGATGGAATGCGGCGCGCCGCGC | |
| | AGCCAAGGTCAGCGCCCGAAAAAGAAACGTA | |
| | AAGTGCATCACCATCACCATCATTAACTCGAG | |
| TRV-R-001 gene | CCATGGCGCACCCTTAGCGAGAGGTTTATCATT | SEQ ID |
| sequence | AAGGTCAACCTCTGGATGTTGTTTCGGCATCCT | NO: 33 |
| | GCATTGAATCTGAGTTACTGTCTGTTTTCCTTG | |
| | TTGGAACGGAGAGCATCACACGACGCTCTTCC | |
| | GATCTAAGTACCCTCAGATCGGAAGAGCACAC | |
| | GTCTGGTAATACTTGATGCTCTCCGAGCCAACC | |
| | AGGAAACCCGTTTTTTCTGACGTAAGGGTGCG | |
| | CAACTTTCATGAAATCCGCTGAATATTTGAACA | |
| | CTTTTAGATTGAGAAATCTCGGCCTACCTGTCA | |
| | TGAACAATTTGCATGACATGTCTAAGGCGACT | |
| | CGCATATCTGTTGAAACACTTCGGTTGTTAATC | |
| | TATACAGCTGATTTTCGCTATAGGATCTACACT | |
| | GTAGAAAAGAAAGGCCCAGAGAAGAGAATGA | |
| | GAACCATTTACCAACCTTCTCGAGAACTTAAA | |
| | GCCTTACAAGGATGGGTTCTACGTAACATTTTA | |
| | GATAAACTGTCGTCATCTCCTTTTTCTATTGGA | |
| | TTTGAAAAGCACCAATCTATTTTGAATAATGCT | |
| | ACCCCGCATATTGGGGCAAACTTTATACTGAA | |
| | TATTGATTTGGAGGATTTTTTCCCAAGTTTAAC | |
| | TGCTAACAAAGTTTTTGGAGTGTTCCATTCTCT | |
| | TGGTTATAATCGACTAATATCTTCAGTTTTGAC | |
| | AAAAATATGTTGTTATAAAAATCTGCTACCAC | |
| | AAGGTGCTCCATCATCACCTAAATTAGCTAATC | |
| | TAATATGTTCTAAACTTGATTATCGTATTCAGG | |
| | GTTATGCAGGTAGTCGGGGCTTGATATATACG | |
| | AGATATGCCGATGATCTCACCTTATCTGCACAG | |
| | TCTATGAAAAAGGTTGTTAAAGCACGTGATTTT | |
| | TTATTTTCTATAATCCCAAGTGAAGGATTGGTT | |
| | ATTAACTCAAAAAAAACTTGTATTAGTGGGCC | |
| | TCGTAGTCAGAGGAAAGTTACAGGTTTAGTTA | |
| | TTTCACAAGAGAAAGTTGGGATAGGTAGAGAA | |
| | AAATATAAAGAAATTAGAGCAAAGATACATCA | |
| | TATATTTTGCGGTAAGTCTTCTGAGATAGAACA | |
| | CGTTAGGGGATGGTTGTCATTTATTTTAAGTGT | |
| | GGATTCAAAAAGCCATAGGAGATTAATAACTT | |
| | ATATTAGCAAATTAGAAAAAAAATATGGAAAG | |
| | AACCCTTTAAATAAAGCGAAGACCTAAGCGGC | |
| | CGC | |
| TRV-R-002 gene | CCATGGAAATCCGCTGAATATTTGAACACTTTT | SEQ ID |
| sequence | AGATTGAGAAATCTCGGCCTACCTGTCATGAA | NO: 34 |
| | CAATTTGCATGACATGTCTAAGGCGACTCGCA | |
| | TATCTGTTGAAACACTTCGGTTGTTAATCTATA | |
| | CAGCTGATTTTCGCTATAGGATCTACACTGTAG | |
| | AAAAGAAAGGCCCAGAGAAGAGAATGAGAAC | |
| | CATTTACCAACCTTCTCGAGAACTTAAAGCCTT | |
| | ACAAGGATGGGTTCTACGTAACATTTTAGATA | |
| | AACTGTCGTCATCTCCTTTTTCTATTGGATTTG | |
| | AAAAGCACCAATCTATTTTGAATAATGCTACC | |
| | CCGCATATTGGGGCAAACTTTATACTGAATATT | |
| | GATTTGGAGGATTTTTTCCCAAGTTTAACTGCT | |
| | AACAAAGTTTTTGGAGTGTTCCATTCTCTTGGT | |
| | TATAATCGACTAATATCTTCAGTTTTGACAAAA | |
| | ATATGTTGTTATAAAAATCTGCTACCACAAGGT | |
| | GCTCCATCATCACCTAAATTAGCTAATCTAATA | |
| | TGTTCTAAACTTGATTATCGTATTCAGGGTTAT | |
| | GCAGGTAGTCGGGGCTTGATATATACGAGATA | |
| | TGCCGATGATCTCACCTTATCTGCACAGTCTAT | |
| | GAAAAAGGTTGTTAAAGCACGTGATTTTTTATT | |
| | TTCTATAATCCCAAGTGAAGGATTGGTTATTAA | |
| | CTCAAAAAAAACTTGTATTAGTGGGCCTCGTA | |
| | GTCAGAGGAAAGTTACAGGTTTAGTTATTTCA | |
| | CAAGAGAAAGTTGGGATAGGTAGAGAAAAAT | |
| | ATAAAGAAATTAGAGCAAAGATACATCATATA | |
| | TTTTGCGGTAAGTCTTCTGAGATAGAACACGTT | |
| | AGGGGATGGTTGTCATTTATTTTAAGTGTGGAT | |
| | TCAAAAAGCCATAGGAGATTAATAACTTATAT | |
| | TAGCAAATTAGAAAAAAAATATGGAAAGAAC | |
| | CCTTTAAATAAAGCGAAGACCTAAACTTTCAG | |
| | ATTCCGTATGCGCACCCTTAGCGAGAGGTTTAT | |
| | CATTAAGGTCAACCTCTGGATGTTGTTTCGGCA | |
| | TCCTGCATTGAATCTGAGTTACTGTCTGTTTTC | |
| | CTTGTTGGACACGACGCTCTTCCGATCTAAGTA | |
| | CCCTCAGATCGGAAGAGCACACGTCTGGTAAT | |
| | ACTTCCAACCAGGAAACCCGTTTTTTCTGACGT | |
| | AAGGGTGCGCATACGGAATCTGCGGCCGC | |
| Exemplary flexible | (Gly-Gly-Gly-Gly-Ser)n | SEQ ID |
| linker | | NO: 35 |
| Exemplary rigid | (EAAAK)n | SEQ ID |
| linker | | NO: 36 |
| PCV target sequence | AAGTATTACC | SEQ ID |
| | | NO: 37 |
| Retron TRV-R-003 | ATGCGCACCCTTAGCGAGAGGTTTATCATTAAGGT | SEQ ID |
| | CAACCTCTGGATGTTGTTTCGGCATCCTGCATTGA | NO: 38 |
| | ATCTGAGTTACTGTCTGTTTTCCTTGTTGGAACG | |
| | GAGAGCATCacacgacgctcttccgatctaagtaccctcagatcgg | |
| | aagagcacacgtctggtaatacttGATGCTCTCCGAGCCAAC | |
| | CAGGAAACCCGTTTTTTCTGACGTAAGGGTGCGC | |
| | AACTTTCATGAAATCCGCTGAATATTTGAACAC | |
| | TTTTAGATTGAGAAATCTCGGCCTACCTGTCAT | |
| | GAACAATTTGCATGACATGTCTAAGGCGACTC | |
| | GCATATCTGTTGAAACACTTCGGTTGTTAATCT | |
| | ATACAGCTGATTTTCGCTATAGGATCTACACTG | |
| | TAGAAAAGAAAGGCCCAGAGAAGAGAATGAG | |
| | AACCATTTACCAACCTTCTCGAGAACTTAAAG | |
| | CCTTACAAGGATGGGTTCTACGTAACATTTTAG | |
| | ATAAACTGTCGTCATCTCCTTTTTCTATTGGAT | |
| | TTGAAAAGCACCAATCTATTTTGAATAATGCTA | |
| | CCCCGCATATTGGGGCAAACTTTATACTGAAT | |
| | ATTGATTTGGAGGATTTTTTCCCAAGTTTAACT | |
| | GCTAACAAAGTTTTTGGAGTGTTCCATTCTCTT | |
| | GGTTATAATCGACTAATATCTTCAGTTTTGACA | |
| | AAAATATGTTGTTATAAAAATCTGCTACCACA | |
| | AGGTGCTCCATCATCACCTAAATTAGCTAATC | |
| | TAATATGTTCTAAACTTGATTATCGTATTCAGG | |
| | GTTATGCAGGTAGTCGGGGCTTGATATATAC | |
| | GAGATATGCCGATGATCTCACCTTATCTGCACA | |
| | GTCTATGAAAAAGGTTGTTAAAGCACGTGATT | |
| | TTTTATTTTCTATAATCCCAAGTGAAGGATTGG | |
| | TTATTAACTCAAAAAAAACTTGTATTAGTGGG | |
| | CCTCGTAGTCAGAGGAAAGTTACAGGTTTAGT | |
| | TATTTCACAAGAGAAAGTTGGGATAGGTAGAG | |
| | AAAAATATAAAGAAATTAGAGCAAAGATACAT | |
| | CATATATTTTGCGGTAAGTCTTCTGAGATAGAA | |
| | CACGTTAGGGGATGGTTGTCATTTATTTTAAGT | |
| | GTGGATTCAAAAAGCCATAGGAGATTAATAAC | |
| | TTATATTAGCAAATTAGAAAAAAAATATGGAA | |
| | AGAACCCTTTAAATAAAGCGAAGACCTAA | |
| F Primer for qPCR | AGACGTGTGCTCTTCCGATCT | SEQ ID |
| (for confirming the | | NO: 39 |
| presence of TRV-D- | | |
| 001) | | |
| R Primer for qPCR | ACACGACGCTCTTCCGATCT | SEQ ID |
| (for confirming the | | NO: 40 |
| presence of TRV-D- | | |
| 001) | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An expression construct comprising: (i) a nucleic acid sequence encoding for a fusion protein, wherein the fusion protein comprises a protein of interest (POI) and a DNA binding protein (DBP) that is fused to the POI; (ii) a nucleic acid sequence encoding for a single-stranded DNA (ssDNA), wherein the ssDNA comprises an ssDNA recognition sequence corresponding to the DBP, and a unique ssDNA barcode corresponding to the POI; and (iii) one or more promoters to drive expression of the fusion protein and the ssDNA.

2. The expression construct of claim 1, wherein the DBP is fused to the N-terminal or C-terminal of the POI, optionally with a linker therebetween.

3. The expression construct of claim 1 or 2, wherein the DBP is a HUH endonuclease.

4. The expression construct of any one of claims 1-3, wherein the DBP comprises an amino acid sequence having at least 85% or 90% sequence identity to the amino acid sequence set forth in any one of SEQ ID NOs: 1-7.

5. The expression construct of any one of claims 1-4, wherein the POI comprises an antibody or antigen-binding fragment thereof, preferably an antigen-binding fragment (Fab) comprising a heavy chain variable (VH) domain and/or a light chain variable (VL) domain.

6. The expression construct of claim 5, wherein the antibody or antigen-binding fragment thereof comprises a VH domain comprising an amino acid sequence having at least 85% or 90% sequence identity to the amino acid sequence set forth in SEQ ID NO: 27 or 29; and/or a VL domain comprising an amino acid sequence having at least 85% sequence identity to the amino acid sequence set forth in SEQ ID NO: 28 or 30.

7. The expression construct of any one of claims 1-6, wherein the nucleic acid sequence encoding a ssDNA further comprises a non-coding RNA (ncRNA), wherein the nucleic acid sequence encoding the ssDNA is inserted into the ncRNA, and wherein the expression construct optionally further comprises a sequence encoding a reverse transcriptase (RT) that is compatible with the ncRNA.

8. The expression construct of claim 7, wherein the ncRNA comprises a stem loop structure, and sequence encoding the ssDNA is inserted into the stem loop structure.

9. The expression construct of claim 7 or 8, wherein the ncRNA and RT is derived from Retron-Eco1 (Ec86), Retron-Eco2 (Ec67), Retron-Eco3 (Ec73), Retron-Eco4 (Ec83), Retron-Eco5 (Ec107), Retron-Eco6 (Ec48), Retron-Eco7 (Ec78), Retron-Mxa1 (Mx162), Retron-Mxa2 (Mx65), Retron-Sau1 (Sa163), Retron-Nex1 (Ne160), Retron-Nex2 (Ne144), Retron-Sen1 (Se72), Retron-Sen2 (St85), Retron-Vch1 (Vc95), Retron-Vch2 (Vc81), Retron-Vch3 (Vc137), Retron-Vpa1 (Vp96), Retron-Kpn1, Retron-Pmi1, Retron-Rxx1, Retron-Bxx1, Retron-Fel1, Retron-Cco1, Retron-Adi1, Retron-Age1, Retron-Cfe1, Retron-Cfu1, Retron-Cvi1, Retron-Mli1, Retron-Mco1, Retron-Mfu1, Retron-Mma1, Retron-Mst1, Retron-Mvi1, Retron-Cap1, Retron-Cpe1, or Retron-Sce1.

10. The expression construct of any one of claims 7-9, wherein the expression construct comprises the sequence encoding the RT at the 5′ or 3′ end of the ncRNA.

11. The expression construct of any one of claims 7-10, wherein the ncRNA comprises nucleotides 6-116 and nucleotides 178-229 of SEQ ID NO: 33, optionally in a first part comprising nucleotides 6-116 and a second part comprising nucleotides 178-229 of SEQ ID NO: 33, wherein the nucleic acid sequence encoding the ssDNA is inserted into the ncRNA between the first and second parts.

12. The expression construct of any one of claims 7-10, wherein the ncRNA comprises nucleotides 2-113 and nucleotides 175-227 of SEQ ID NO: 38, optionally in a first part comprising nucleotides 2-113 and a second part comprising nucleotides 175-227 of SEQ ID NO: 38, wherein the nucleic acid sequence encoding the ssDNA is inserted into the ncRNA between the first and second parts.

13. The expression construct of any one of claims 7-12, wherein the sequence encoding the RT comprises nucleotides 237-1199 of SEQ ID NO: 33 or nucleotides 234-1196 of SEQ ID NO: 38.

14. The expression construct of any one of claims 1-13, wherein the expression construct comprises nucleotides 6-116, nucleotides 178-229, and/or nucleotides 237-1199 of SEQ ID NO: 33.

15. The expression construct of any one of claims 1-13, wherein the expression construct comprises nucleotides 2-113, nucleotides 175-227, and/or nucleotides 234-1196 of SEQ ID NO: 38.

16. The expression construct of any one of claims 1-15, wherein the ssDNA recognition sequence comprises at least 85% or 90% sequence identity to the nucleic acid sequence set forth in any one of SEQ ID NOs: 8-14.

17. The expression construct of any one of claims 1-16, wherein the ssDNA barcode comprises a nucleic acid sequence having at least 85% or 90% sequence identity to the nucleic acid sequence set forth in any one of SEQ ID NOs: 15-19.

18. The expression construct of any one of claims 1-17, wherein the expression construct comprises one single promoter to drive expression of the fusion protein and the ssDNA; or two promoters, wherein a first promoter drives expression of the fusion protein and a second promoter drives expression of the ssDNA.

19. The expression construct of claim 18, wherein the first and/or second promoter is selected from the group consisting of T7, T7lac, lac, Sp6, araBAD, trp, Ptac, pL, T3CMV, SV40, EF1a, PGK1, Ubc, human beta actin, CAG, TRE, UAS, Ac5, POlyhedrin, CaMKIIa, GAL-1,10, TEF1, GDS, ADH1, CaMV35S, H1, and U6.

20. The expression construct of any one of claims 1-19, wherein the expression construct further comprise a nucleic acid sequence encoding for a purification tag.

21. The expression construct of claim 20, wherein the purification tag is a His-tag.

22. The expression construct of claim 20, wherein the purification tag is a FLAG tag or a biotin-tag.

23. The expression construct of any one of claims 1-22, wherein the expression construct comprises a nucleic acid sequence having at least 85% sequence identity to the nucleic acid sequence set forth in SEQ ID NO: 33 or 38.

24. An isolated cell comprising the expression construct of any one of claims 1-23.

25. The cell of claim 24, wherein the cell is a prokaryotic cell or a eukaryotic cell.

26. The cell of claim 25, wherein the cell is Escherichia coli, Saccharomyces cerevisiae, an insect cell, or a mammalian cell.

27. A method of generating a DNA encoded polypeptide (DEP), the method comprising:
(a) transforming an expression construct of any one of claims 1-23 into cells under conditions in which one expression construct is introduced into each cell; and
(b) culturing the cells under conditions in which the expression construct is expressed, and the DBP of the fusion protein binds to the corresponding ssDNA recognition sequence, thereby producing a DEP.

28. The method of claim 27, wherein the cell is a prokaryotic cell or a eukaryotic cell.

29. The method of claim 28, wherein the cell is Escherichia coli, Saccharomyces cerevisiae, or a mammalian cell.

30. The method of any one of claims 27-29, further comprising purifying the DEP.

31. The method of claim 30, wherein the purifying step comprises pulling down the DEP with a pull-down assay, wherein the pull-down assay is compatible with the purification tag that is encoded by the expression construct.

32. A method of any one of claims 27-31, wherein the DEP comprises the fusion protein and the ssDNA, wherein the fusion protein is conjugated to the ssDNA by a covalent bond or a non-covalent bond.

33. The method of claim 32, wherein the covalent bond or the non-covalent bond is between the DBP of the fusion protein and its corresponding ssDNA recognition sequence.

34. The method of any one of claims 27-33, wherein the method further comprises identifying the POI of the fusion protein.

35. The method of claim 34, wherein the identifying step comprises sequencing the ssDNA barcode.

36. A DEP generated by the method of any one of claims 27-35.

37. A composition comprising the DEP of claim 36.

38. The composition of claim 37, further comprising a pharmaceutically acceptable carrier.

39. A method of generating a DEP library, the method comprising:
(a) transforming a pool of expression constructs into cells under conditions in which one expression construct is introduced into each cell, wherein the pool of expression constructs comprises a plurality of the expression construct of any one of claims 1-23; and
(b) culturing the cells under conditions in which the pool of expression constructs are expressed, and the DBP of the fusion proteins bind to the corresponding ssDNA recognition sequences, thereby producing a DEP library.

40. A DEP library generated by the method of claim 39.

41. The DEP library of claim 40, wherein the DEP library comprise about 10² to about 10¹⁴ DEPs.

42. A DEP from the library of claim 40 or 41, wherein the DEP comprises a fusion protein conjugated to an ssDNA, wherein
the fusion protein comprises a POI variant and a DBP fused to the POI variant;
the ssDNA comprises an ssDNA recognition sequence corresponding to the DBP, and a unique ssDNA barcode corresponding to the POI variant; and
the DBP of the fusion protein is conjugated to the ssDNA recognition sequence of the ssDNA by a covalent bond or a non-covalent bond.

43. A method for selecting a polypeptide variant as a candidate delivery vehicle, the method comprising:
(a) generating a DEP library by the method of claim 39, wherein the pool of expression constructs comprises nucleic acid sequences encoding for a pool of polypeptide variants, and wherein each DEP of the DEP library comprises a fusion protein conjugated to an ssDNA, wherein the fusion protein comprises a polypeptide variant and a DBP fused to the polypeptide variant, and the ssDNA comprises an ssDNA recognition sequence corresponding to the DBP and a unique ssDNA barcode corresponding to the polypeptide variant;
(b) administering the DEP library generated in step (a) to an animal;
(c) obtaining a biological sample from the animal;
(d) processing the biological sample by homogenization or subcellular fractionation to extract the DEP;
(e) next generation sequencing to identify the ssDNA barcodes and the corresponding polypeptide variants;
(f) screening a tissue and/or cellular fraction and/or subcellular fraction of interest obtained in step (d) to determine abundance of ssDNA barcodes and the corresponding polypeptide variants therein; and
(g) selecting a polypeptide variant as a candidate delivery vehicle based on abundance of ssDNA barcode specific for the polypeptide variant in the tissue and/or cellular fraction and/or subcellular fraction of interest.

44. The method of claim 43, wherein the animal is a non-human mammal.

45. The method of claim 44, wherein the animal is a non-human primate, a mouse, a rat, a rabbit, a mini-pig, a sheep, or a dog.

46. The method of any one of claims 43-45, wherein the administration to the animal is by enteral administration, parenteral administration, intranasal administration, administration by inhalation, or vaginal administration.

47. The method of claim 46, wherein the enteral administration is by intravenous injection, intramuscular injection, subcutaneous injection, topical administration, or transdermal administration.

48. The method of any one of claims 43-47, wherein the biological sample is a tissue, blood and/or plasma.

49. An in vitro method for selecting a polypeptide variant as a candidate delivery vehicle, the method comprising:
(a) generating a DEP library by the method of claim 39, wherein the pool of expression constructs comprises nucleic acid sequences encoding for a pool of polypeptide variants, and wherein each DEP of the DEP library comprises a fusion protein conjugated to an ssDNA, wherein the fusion protein comprises a polypeptide variant and a DBP fused to the polypeptide variant, and the ssDNA comprises an ssDNA recognition sequence corresponding to the DBP and a unique ssDNA barcode corresponding to the polypeptide variant;
(b) dosing the DEP library to a cell culture;
(c) processing the cell culture by homogenization or subcellular fractionation to extract the DEP;
(e) next generation sequencing to identify the ssDNA barcodes and the corresponding polypeptide variants;
(f) screening a cellular fraction and/or subcellular fraction of interest obtained in step (c) to determine abundance of ssDNA barcodes and the corresponding polypeptide variants therein; and
(g) selecting a polypeptide variant as a candidate delivery vehicle based on abundance of ssDNA barcode specific for the polypeptide variant in the cellular fraction and/or subcellular fraction of interest.

50. The method of any one of claims 43-49, wherein the polypeptide variant is selected as a candidate delivery vehicle for a bio-therapeutic.

51. The method of claim 50, wherein the bio-therapeutic is an antisense oligonucleotides (ASO) and/or a small interfering RNA (siRNA).

52. The method of any one of claims 43-51, wherein the polypeptide variant is a variant of a GLP1R (glucagon like peptide 1 receptor) binder, a DPP6 (dipeptidyl peptidase like 6) binder, and/or a CCK-2 (cholecystokinin-2 receptor) binder.

53. A method for selecting a polypeptide variant as a candidate bio-therapeutic, the method comprising:
(a) generating a DEP library by the method of claim 39, wherein the pool of expression constructs comprises nucleic acid sequences encoding for a pool of polypeptide variants, and wherein each DEP of the DEP library comprises a fusion protein conjugated to an ssDNA, wherein the fusion protein comprises a polypeptide variant and a DBP fused to the polypeptide variant, and the ssDNA comprises an ssDNA recognition sequence corresponding to the DBP and a unique ssDNA barcode corresponding to the polypeptide variant;
(b) administering the DEP library generated in step (a) to an animal;
(c) obtaining plasma sample from the animal at a specific time-point after administration of the DEP library;
(d) processing the plasma sample by homogenization to extract the DEP;
(e) next generation sequencing to identify the ssDNA barcodes and the corresponding polypeptide variants;
(f) screening the homogenized plasma sample obtained in step (d) to determine abundance of ssDNA barcodes and the corresponding polypeptide variants in the plasma; and
(g) selecting a polypeptide variant as a candidate bio-therapeutic based on abundance of ssDNA barcode specific for the polypeptide variant in the plasma.

54. The method of claim 53, wherein the animal is a non-human mammal.

55. The method of claim 54, wherein the animal is a non-human primate, a mouse, a rat, a rabbit, a mini-pig, a sheep, or a dog.

56. The method of any one of claims 53-55, wherein the administration to the animal is by enteral administration, parenteral administration, intranasal administration, administration by inhalation, or vaginal administration.

57. The method of claim 56, wherein the enteral administration is by intravenous injection, intramuscular injection, subcutaneous injection, topical administration, or transdermal administration.
